(19) United States
(12) Patent Application Publication
(10) Pub. No.: US 2024/0398962 A1
(43) Pub. Date: Dec. 5, 2024

(54) DENDRIMER-GLUTAMINE ANTAGONIST CONJUGATES AND METHODS OF USE THEREOF

(71) Applicants: The Johns Hopkins University, Baltimore, MD (US); Institute of Organic Chemistry and Biochemistry, Prague 6 (CZ)

(72) Inventors: Benjamin Bell, Baltimore, MD (US); Rana Rais, Baltimore, MD (US); Kannan Rangaramanujam, Baltimore, MD (US); Anjali Sharma, Baltimore, MD (US); Barbara Slusher, Baltimore, MD (US); Takashi Tsukamoto, Baltimore, MD (US); Run-Duo Gao, Baltimore, MS (US); Pavel Majer, Prague 6 (CZ); Katerina Novotna, Prague 6 (CZ); Lukas Tenora, Prague 6 (CZ); Martin Hadzima, Prague 6 (CZ); Martin Kaiser, Prague 6 (CZ); Thomas Tichy, Prague 6 (CZ); Xiaolei Zhu, Baltimore, MD (US)

(21) Appl. No.: 18/554,946

(22) PCT Filed: Apr. 14, 2022
(86) PCT No.: PCT/US2022/024752

§ 371 Date: Oct. 11, 2023

Related U.S. Application Data
(60) Provisional application No. 63/174,878, filed on Apr. 14, 2021.

(51) Int. Cl. A61K 47/59; A61P 35/00; C07C 245/18; C07F 9/22

(52) U.S. Cl. CPC A61K 47/59; A61P 35/00; C07C 245/18; C07F 9/222

(57) ABSTRACT

Compositions of dendrimers conjugated with one or more glutamine antagonist(s) that inhibit glutamine metabolism, preferably in activated microglia, and methods of use thereof for treating, alleviating, and/or preventing one or more neurological, oncological, and/or immune disorders associated with pathogenic or dysregulated glutamine-dependent pathways and/or glutamate transmission, have been developed. Dendrimers conjugated with one or more glutamine antagonists effectively inhibit the activity of glutaminase without any systemic toxicity. Dendrimers conjugated with one or more glutamine antagonists accumulate in activated microglia associated with the injured or diseased cells and tissues.

### CROSS-REFERENCE TO RELATED APPLICATIONS

This application claims priority to and benefit of U.S. Provisional Application No. 63/174,878, filed Apr. 14, 2021, which is hereby incorporated herein by reference in its entirety.

### FIELD OF THE INVENTION

The invention is generally in the field of drug delivery, and in particular, glutamine antagonists bound via dendrimers for selective uptake within sites or regions of diseased and injured cells and tissues in need thereof.

### BACKGROUND OF THE INVENTION

The broadly active glutamine antagonist 6-diazo-5-oxo-L-norleucine (DON) has been studied for sixty years as a potential anti-cancer therapeutic. Studies have shown that DON has robust anti-cancer potential, including in treatment of brain cancers. It has also been demonstrated that DON can block glutamate release from activated microglia, normalize glutamate production in activated immune cells, and provide broad therapeutic utility in neurological, neuropsychiatric, and immune disorders where neuroinflammation and excitotoxicity is involved.

Despite the therapeutic potential of DON, it has not been clinically developed due to its systemic toxicities, most of which are gastrointestinal (GI)-related. To get adequate therapeutic concentrations of DON into the target tissues, the peripherally administered dose must be high, leading to intolerable GI side effects. Thus, there is a need for new glutamine antagonists having similar biological activities and therapeutic potential as DON, but with reduced or minimal systemic toxicities.

Therefore, it is an object of the invention to provide compositions for reducing and preventing pathological processes associated with dysregulated glutamine-dependent pathways and/or glutamate transmission, and methods of making and using thereof.

It is also an object of the invention to provide compositions for the treatment or prevention of a variety of CNS, oncological, and immune disorders where dysregulated glutamine-dependent pathways and/or glutamate transmission are presumed pathogenic.

### SUMMARY OF THE INVENTION

Compositions of 6-diazo-5-oxo-L-norleucine (DON) analogues have been developed.

In some other instances, DON analogs may be used which have one of the following three general chemical formulae, wherein “AA” refers to an amino acid:

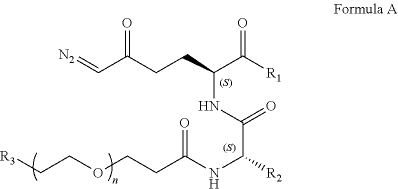

where R₁ is selected from —OCH₂CH₃, —OCH(CH₃)₂—OC(CH₃)₃, —NH₂, NHCH₃, —NH-AA-COOCH(CH₃)₂, and —NH-AA-COOC(CH₃)₃;
where R₂ is selected from —H, —CH₃, —CH₂phenyl, —CH₂CH(CH₃)₂, —CH₂ (indol-3-yl), and —CH₂CH₂C(═O) CH═N≡N;
where R₃ is selected from —NH₂ and —OH; and
n is an integer value from 1 to 32,

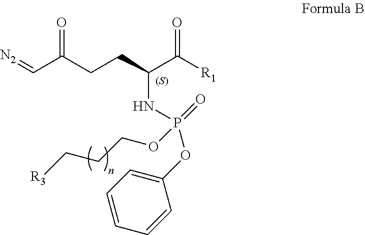

where R₁ is selected from —OCH₂CH₃, —OCH(CH₃)₂—OC(CH₃)₃, —NH₂, NHCH₃, —NH-AA-COOCH(CH₃)₂, and —NH-AA-COOC(CH₃)₃;
where R₃ is selected from —NH₂ and —OH; and
n is an integer value from 1 to 16,

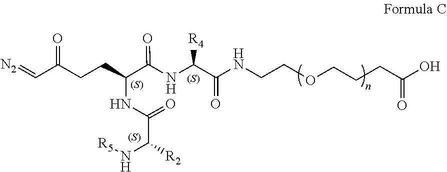

where R₂ is selected from —H, —CH₃, —CH₂phenyl, —CH₂CH(CH₃)₂, —CH₂ (indol-3-yl), or —CH₂CH₂C(═O) CH═N≡N;
where R₄ is selected from —H, —CH₃, —CH₂phenyl, or —CH₂CH(CH₃)₂;
where R₅ is selected from —H, —Ac, —Fmoc, dimethylglycine, Ac-AA-C(═O)—, dimethylglycine-NH-AA-C(═O)—, dimethylglycine-NH-DON-C(═O)—, or C₈H₁₇O—C(═O)CH(NH₂)CH₂CH₂C(═O)—NH—6-diazo-5-oxo-L-norleucine —C(═O)—; and
n is an integer value from 1 to 32.
DON analogues include molecules having the structure of any one of structures I-X:

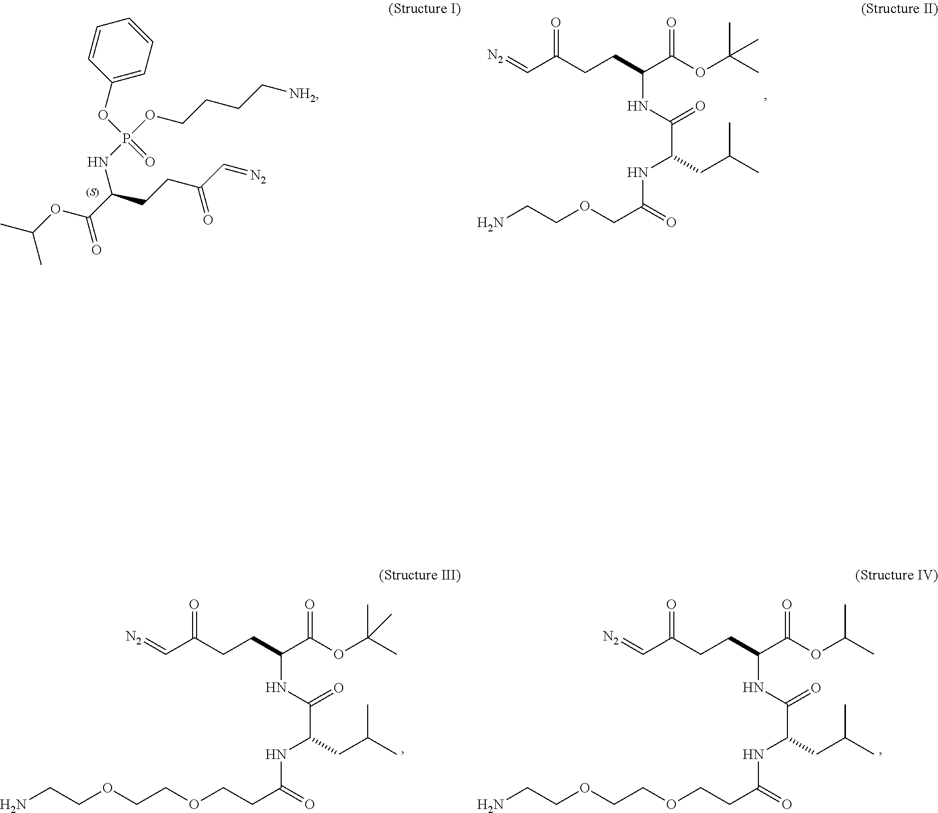

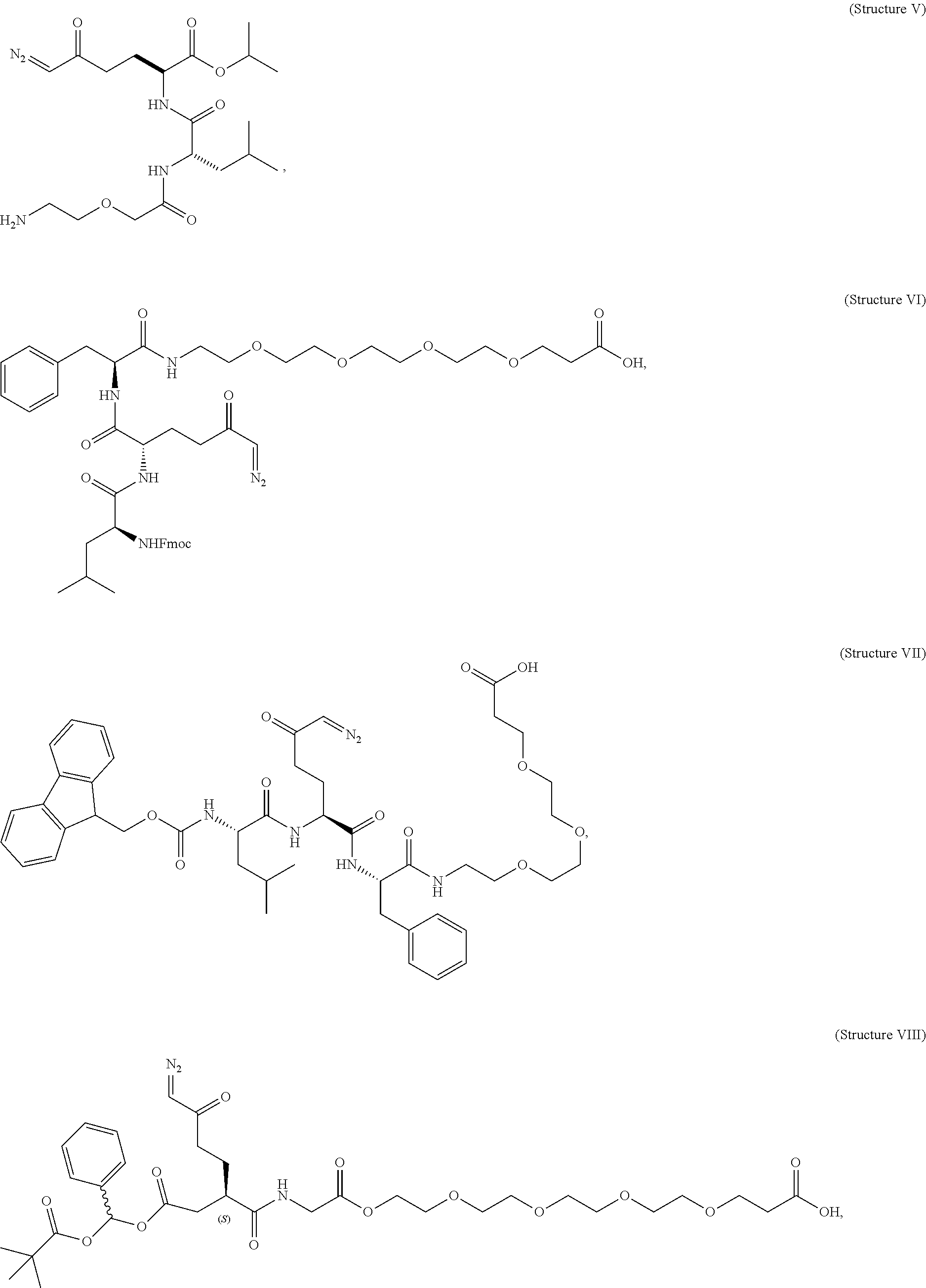

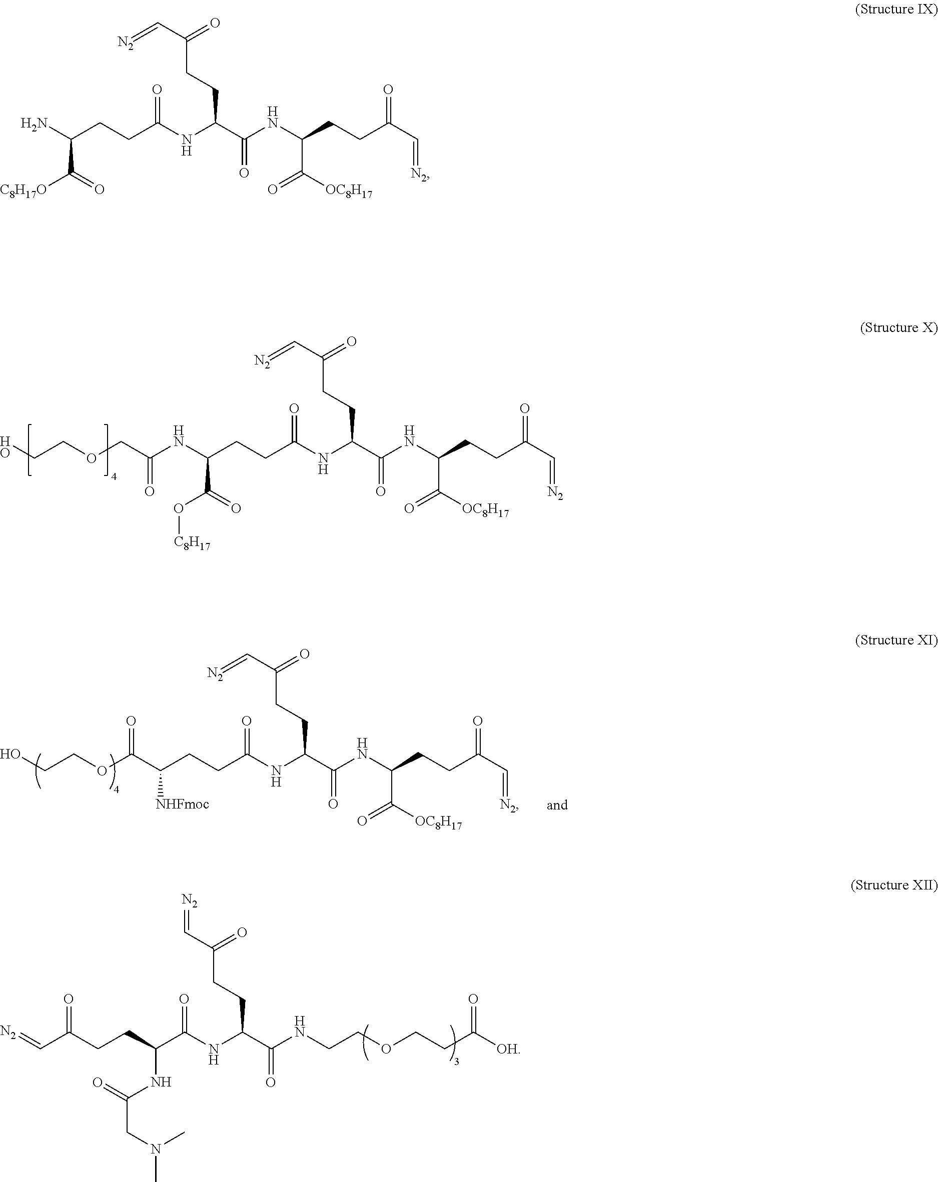

Compositions of dendrimers conjugated to one or more DON analogues are also described. Compositions including dendrimers complexed, covalently conjugated, or intra-molecularly dispersed or encapsulated with one or more 6-diazo-5-oxo-L-norleucine (DON) analogs having any of structures I-X or general Formulae A-C are provided.

Exemplary dendrimers include generation 4, generation 5, generation 6, generation 7, or generation 8 dendrimers. In some embodiments the dendrimers are poly(amidoamine) (PAMAM) dendrimers, for example, hydroxyl-terminated PAMAM dendrimers. In a preferred embodiment, the dendrimers are generation 4, generation 5, or generation 6, hydroxyl-terminated PAMAM dendrimers.

In some embodiments, the dendrimers are covalently conjugated to one or more DON analogs, optionally via a linker or spacer moiety. In some embodiments, the dendrimers are further complexed or conjugated with one or more therapeutic, prophylactic, or diagnostic agents. Exemplary therapeutic agents include neuroprotective agents, anti-inflammatory agents, and/or chemotherapeutic agents.

Pharmaceutical compositions, including dendrimers complexed, covalently conjugated, or intra-molecularly dispersed or encapsulated with one or more 6-diazo-5-oxo-L-norleucine (DON) analogs having any of structures I-XII or Formulae A-C, and one or more pharmaceutically acceptable excipients are also provided.

The pharmaceutical compositions are formulated for parenteral or oral administration, for example, formulated in a form of hydrogels, nanoparticles or microparticles, suspensions, powders, tablets, capsules, suspensions, and solutions.

Methods for treating one or more of neurological, oncological, and/or immune disorders in a subject in need thereof, include administering to the subject an effective amount of dendrimers complexed, covalently conjugated, or intra-molecularly dispersed, or encapsulated with one or more 6-diazo-5-oxo-L-norleucine (DON) analogs having any of structures I-XII or general Formulae A-C, to treat, alleviate, and/or prevent one or more symptoms associated with the one or more of neurological, oncological, and immune disorders. Preferably, the composition is administered in an amount effective to decrease glutaminase activity in diseased or injured tissue of one or more of neurological, oncological, and immune disorders. For example, in some embodiments, the composition is administered in an amount effective to decrease glutaminase activity in activated microglia associated with the diseased and/or injured tissue of one or more of neurological, oncological, and immune disorders. Exemplary neurological disorders that can be treated with these DON analogs include stress-induced mood disorders, cognitive deficit, ischemia, neuroinflammation, Alzheimer's disease, and Multiple Sclerosis. In one embodiment, the disease or disorder is a stress-induced psychiatric disorder such as depression. In some embodiments, the composition is administered in an amount effective to reduce neuroinflammation, improve cognition, or a combination thereof. Exemplary oncological disorders that can be treated by the methods include breast cancer, ovarian cancer, uterine cancer, prostate cancer, testicular germ cell tumor, brain cancer, gastric cancer, esophagus cancer, lung cancer, liver cancer, renal cell cancer, colorectal cancer, and pancreatic cancer. In some embodiments, the composition is administered in an amount effective to reduce tumor size. Exemplary immune disorders that can be treated by the methods include rheumatoid arthritis, psoriasis, psoriatic arthritis, systemic lupus erythematosus (SLE), type 1 diabetes, inflammatory bowel disease, and thyroid diseases. In further embodiments, the composition is administered to the subject together with one or more of an immune checkpoint modulator, a chemotherapeutic agent, an anti-infective agent, adoptive T cell therapy, a cancer vaccine, surgery, and radiation therapy. Exemplary immune checkpoint modulators include PD-1 antagonists, PD-1 ligand antagonists, and CTLA4 antagonists.

Methods for making one or more 6-diazo-5-oxo-L-norleucine (DON) analogs having any of structures I-X or Formulae A-C and methods of conjugating to dendrimers are also described.

### BRIEF DESCRIPTION OF THE DRAWINGS

FIG. 1 is a schematic representation of the synthesis of compound I.

FIG. 2 is a schematic representation of the synthesis of compound II.

FIG. 3 is a schematic representation of the synthesis of compound III.

FIG. 4 is a schematic representation of the synthesis of compound IV.

FIG. 5 is a schematic representation of the synthesis of compound V.

FIG. 6 is a schematic representation of the synthesis of compound VI.

FIG. 7 is a schematic representation of the synthesis of compound VII.

FIG. 8 is a schematic representation of the synthesis of compound VIII.

FIG. 9 is a schematic representation of the synthesis of compounds IX and X.

FIG. 10 is a schematic representation of the synthesis of compounds XI.

FIG. 11 is a scheme showing the synthetic scheme for compounds XII.

FIG. 12 is a schematic representation of the general synthetic route for dendrimer-glutamine antagonist conjugates.

FIG. 13 is a schematic showing experimental setup using mice. Mice were injected intra-cranially with LPS (2.5 mg/kg) into their right striatum. Three hours later Dendrimer-TTM020 or empty dendrimer was administered I.P. at a dose of 2 mg/kg DON equivalence. 24 and 72 hours later mice were sacrificed, and right hemisphere was removed and CD11b cells were extracted for glutaminase activity measurement.

FIG. 14 is a schematic drawing showing the chemical structure of dendrimer conjugated to TTM020.

FIG. 15 is a bar graph showing glutaminase activity measured in fmol/mg/h in each of control mice, mice treated with unconjugated dendrimer (D-EMPTY) or DON analogue TTM020 conjugated dendrimer (D-DON), respectively, 24 hours and 72 hours post-drug treatment.

FIG. 16 is a bar graph showing uptake of dendrimer-Cy5 (AUC) in contralateral, peritumor and tumor regions of the brain in a rodent tumor-inoculated 9L gliosarcoma model. n=6.

FIG. 17 is a schematic drawing showing the chemical structure of dendrimer conjugated to DON analog via a linker.

FIGS. 18A and 18B are bar graphs showing tumor mass (FIG. 18A), and tumor burden as a percentage of the brain mass (FIG. 18B) in each of untreated control group, mice treated with unconjugated DON (free DON), and in mice treated with dendrimer KMN-045 conjugate (D-045), respectively, in a GL261 murine model of GBM in C57BL/6 mice.

FIG. 19 is a bar graph showing % DON release over time for in vitro stability of dendrimer KMN-045 conjugate in brain homogenate.

FIG. 20A is a line graph showing the release of DON (μM) in tumor homogenate over a period of 60 minutes. FIG. 20B a line graph showing suppression of tumor volume in mm³ over a period of 14 days in a syngeneic EL4 lymphoma model when treated with D-DON (TTM020) or vehicle. FIG. 20C is a line graph showing percent weight change over a time period of 14 days when treated with D-DON (TTM020) or vehicle.

FIG. 21 is a bar graph showing glutaminase activity (fmol/mg/h) in three experimental groups including control mice treated with vehicle (Ctr+Vehicle), CSDS mice treated with vehicle (CSDS+Vehicle), and CSDS mice treated with D-TTM020 (CSDS+D-TTM020). **p<0.01, comparison between Ctr+Vehicle vs. CSDS+Vehicle. ##p<0.01, comparison between CSDS+Vehicle vs. CSDS+D-TTM020. Analyzed by one-way ANOVA with post hoc Tukey test. n=5 per condition.

### DETAILED DESCRIPTION OF THE INVENTION

### I. Definitions

The terms “active agent” or “biologically agent” refer to therapeutic, prophylactic or diagnostic chemical or biological compounds that induce a desired pharmacological and/or physiological effect, which may be prophylactic, therapeutic or diagnostic. These may be a nucleic acid, a nucleic acid analog, a small molecule having a molecular weight less than 2 kD, more typically less than 1 kD, a peptidomimetic, a protein or peptide, carbohydrate or sugar, lipid, or a combination thereof. The terms also encompass pharmaceutically acceptable, pharmacologically active derivatives of agents, including, but not limited to, salts, esters, amides, prodrugs, active metabolites, and analogs.

The term “therapeutic agent” refers to an agent that can be administered to treat one or more symptoms of a disease or disorder.

The term “diagnostic agent” generally refers to an agent that can be administered to reveal, pinpoint, and define the localization of a pathological process. The diagnostic agents can label target cells that allow subsequent detection or imaging of these labeled target cells. In some embodiments, diagnostic agents can, via dendrimer or suitable delivery vehicles, target/bind activated microglia in the central nervous system (CNS).

The term “prophylactic agent” generally refers to an agent that can be administered to prevent disease, or a symptom thereof, or to prevent certain conditions, such as a vaccine.

The term “analog” refers to a chemical compound with a structure similar to that of another (reference compound) but differing from it in respect to a particular component, functional group, atom, etc. The terms “6-diazo-5-oxo-L-norleucine (DON) analogue”, or “DON analog” refer to a chemical compound with a structure similar to that of 6-diazo-5-oxo-L-norleucine, but differing from it in some minor manner. Exemplary DON analogs include any of the structures I-X or compounds encompassed by general Formulae A-C.

The term “derivative” refers to compounds which are formed from a parent compound by one or more chemical reaction(s).

The term “alkyl” as used herein, means a straight or branched, saturated hydrocarbon chain containing from 1 to 10 carbon atoms, including 1, 2, 3, 4, 5, 6, 7, 8, 9, and 10 carbon atoms. The term “lower alkyl” or “C₁-C₆-alkyl” means a straight or branched chain hydrocarbon containing from 1 to 6 carbon atoms, including 1, 2, 3, 4, 5, and 6 carbon atoms. The term “C₁-C₃-alkyl” means a straight or branched chain hydrocarbon containing from 1 to 3 carbon atoms, including 1, 2, and 3 carbon atoms. Representative examples of alkyl include, but are not limited to, methyl, ethyl, n-propyl, isopropyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylenyl,” as used herein, refers to a divalent group derived from a straight or branched chain hydrocarbon of 1 to 50 carbon atoms, including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50, for example, of 1 to 5 carbon atoms, including 1, 2, 3, 4, and 5 carbon atoms. Representative examples of alkylenyl include, but are not limited to, —CH₂—, —CH₂—CH₂—, —CH₂—CH₂—CH₂—, —CH₂—CH₂—CH₂—CH₂—, and —CH₂—CH₂—CH₂—CH₂—CH₂—.

The term “alkenylenyl,” as used herein, refers to a divalent group derived from a straight or branched chain hydrocarbon of 2 to 50 carbon atoms, including 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 carbon atoms, wherein at least 5 one carbon-carbon bond is a double bond. Representative examples of alkenylenyl include, but are not limited to, —CH═CH—, —CH═CH—CH₂—, —CH₂—CH═CH—CH₂—, and —CH₂CH₂—CH═CH—CH₂—.

The term “alkynylenyl,” as used herein, refers to a divalent group derived from a straight or branched chain hydrocarbon of 2 to 50 carbon atoms, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 carbon atoms, wherein at least one carbon-carbon bond is a triple bond. Representative examples of alkynylenyl include, but are not limited to, —C≡C—, —C≡C—CH₂—, —C≡C—CH₂—CH₂— and —CH₂—C≡C—CH₂—.

The terms “alkoxyl” or “alkoxy,” “aroxy” or “aryloxy,” generally describe compounds represented by the formula —OR^{V}, wherein R^{V} includes, but is not limited to, a substituted or unsubstituted alkyl, a substituted or unsubstituted alkenyl, a substituted or unsubstituted alkynyl, a substituted or unsubstituted cycloalkyl, a substituted or unsubstituted heterocyclyl, a substituted or unsubstituted cycloalkenyl, a substituted or unsubstituted heterocycloalkenyl, a substituted or unsubstituted aryl, a substituted or unsubstituted heteroaryl, a substituted or unsubstituted polyaryl, a substituted or unsubstituted polyheteroaryl, a substituted or unsubstituted arylalkyl, a substituted or unsubstituted heteroalkyl, a substituted or unsubstituted alkylaryl, a substituted or unsubstituted alkylheteroaryl, a substituted or unsubstituted aralkyl, a substituted or unsubstituted carbonyl, a sugar group, a phosphonium, a phosphanyl, a phosphonyl, a sulfinyl, a silyl, a thiol, an amido, and an amino. Exemplary alkoxyl groups include methoxy, ethoxy, propyloxy, tert-butoxy and the like. A lower alkoxy group is an alkoxy group containing from one to six carbon atoms. An ether is two functional groups covalently linked by an oxygen as defined below. Accordingly, the substituent of an alkyl that renders that alkyl an ether is or resembles an alkoxyl, such as can be represented by one of —O-alkyl, —O-alkenyl, —O-alkynyl, —O-aryl, —O-heteroaryl, —O-polyaryl, —O-polyheteroaryl, —O-heterocyclyl, etc.

The term “carbonyl” as used herein, is represented by the general formula:

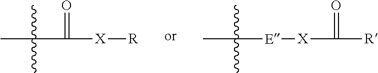

wherein X is a bond, or represents an oxygen or a sulfur, and R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonyl, an amido, an amino, or —(CH₂)ₘ—R″; E″ is absent, or E″ is substituted or unsubstituted alkyl, substituted or unsubstituted alkenyl, substituted or unsubstituted alkynyl, substituted or unsubstituted aralkyl, substituted or unsubstituted aryl, substituted or unsubstituted heteroaryl, a substituted or unsubstituted polyaryl, a substituted or unsubstituted polyheteroaryl, substituted or unsubstituted heterocyclyl; R′ represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonyl, an amido, an amino, or —(CH₂)ₘ—R″; R″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonyl, an amido, or an amino; and m is zero or an integer ranging from 1 to 8. Where X is oxygen and R is defined as above, the moiety is also referred to as a carboxyl group. When X is oxygen and R is hydrogen, the formula represents a “carboxylic acid”. Where X is oxygen and R′ is hydrogen, the formula represents a “formate”. Where X is oxygen and R or R′ is not hydrogen, the formula represents an “ester”. Where X is a bond and R is not hydrogen, the above formula represents a “ketone”. Where X is a bond and R is hydrogen, the above formula represents an “aldehyde”.

The term “phosphoryl”, as used herein, is represented by the general formula:

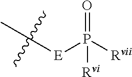

wherein E is absent, oxygen, substituted or unsubstituted alkoxy, or substituted or unsubstituted aroxy, wherein independently of E, R^{vi} and R^{vii} are independently R^{vi} and R^{vii} are independently hydroxyl, substituted or unsubstituted alkoxy, or substituted or unsubstituted aroxy, as defined above. When E, R^{vi} and R^{vii} are substituted, the substituents include, but are not limited to, halogen, azide, azo, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, amino (e.g. quarternized amino), amido, amidine, imine, cyano, nitro, azido, sulfhydryl, alkylthio, sulfonyl, heterocyclyl, alkylaryl, haloalkyl, —CN, aryl, heteroaryl, polyaryl, polyheteroaryl, and combinations thereof.

“Substituted,” as used herein, refers to all permissible substituents of the compounds or functional groups described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an azo, an oxo, a sulfinyl, a sulfonyl, a sulfonic acid, a phosphonium, a phosphanyl, a phosphoryl, a phosphonyl, an amino acid, a polymer, a peptide, and a sugar group. Such a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, an amino acid, a polymer, a peptide, and a sugar group can be further substituted.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

Description of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable” or “biocompatible” refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The term “pharmaceutically acceptable salts” is art-recognized, and includes relatively non-toxic, inorganic and organic acid addition salts of compounds. Examples of pharmaceutically acceptable salts include those derived from mineral acids, such as hydrochloric acid and sulfuric acid, and those derived from organic acids, such as ethanesulfonic acid, benzenesulfonic acid, and p-toluenesulfonic acid. Examples of suitable inorganic bases for the formation of salts include the hydroxides, carbonates, and bicarbonates of ammonia, sodium, lithium, potassium, calcium, magnesium, aluminum, and zinc. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine;

The term “biodegradable” generally refers to a material that will degrade or erode under physiologic conditions to smaller units or chemical species that are capable of being metabolized, eliminated, or excreted by the subject. The degradation time is a function of composition and morphology.

The term “therapeutically effective amount” refers to an amount of the therapeutic agent that, when incorporated into and/or onto dendrimers, produces some desired effect at a reasonable benefit/risk ratio applicable to any medical treatment. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation. In some embodiments, the term “effective amount” refers to an amount of a therapeutic agent or prophylactic agent to reduce or diminish the symptoms of one or more diseases or disorders, such as reducing tumor size (e.g., tumor volume) or reducing or diminishing one or more symptoms of an autoimmune diseases, such as pain and swelling in the wrist and small joints of the hand and feet in patients with rheumatoid arthritis etc. In the case of cancer or tumor, an effective amount of the drug may have the effect of reducing the number of cancer cells; reducing the tumor size; inhibiting cancer cell infiltration into peripheral organs; inhibiting tumor metastasis; inhibiting tumor growth; and/or relieving one or more of the symptoms associated with the disorder. An effective amount can be administered in one or more administrations.

The terms “inhibit” or “reduce” in the context of inhibition, mean to reduce or decrease in activity and quantity. This can be a complete inhibition or reduction in activity or quantity, or a partial inhibition or reduction. Inhibition or reduction can be compared to a control or to a standard level. Inhibition can be 5, 10, 25, 50, 75, 80, 85, 90, 95, 99, or 100%. For example, dendrimer compositions including one or more DON analogs may inhibit or reduce the activity and/or quantity of glutaminase associated activated microglia by about 5%, 10%, 20%, 30%, 40%, 50%, or more than 50% from the activity and/or quantity of the same cells in equivalent tissues of subjects that did not receive, or were not treated with the dendrimer compositions. In some embodiments, the inhibition and reduction are compared at mRNAs, proteins, cells, tissues and organs levels. For example, an inhibition and reduction in the rate of neural loss, in the rate of decrease of brain weight, or in the rate of decrease of hippocampal volume, as compared to an untreated control subject.

The term “treating” or “preventing” a disease, disorder or condition from occurring in an animal which may be predisposed to the disease, disorder and/or condition but has not yet been diagnosed as having the disease, disorder, or condition; inhibiting the disease, disorder or condition, e.g., impeding its progress; and/or relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating or palliating the disease state, and remission or improved prognosis. For example, an individual is successfully “treated” if one or more symptoms associated with cancers are mitigated or eliminated, including, but are not limited to, reducing the rate of neuronal loss, decreasing symptoms resulting from the disease, increasing the quality of life of those suffering from the disease, decreasing the dose of other medications required to treat the disease, delaying the progression of the disease, and/or prolonging survival of individuals.

The term “dendrimer” includes, but is not limited to, a molecular architecture with an interior core, interior layers (or “generations”) of repeating units regularly attached to this initiator core, and an exterior surface of terminal groups attached to the outermost generation.

The term “functionalize” means to modify a compound or molecule in a manner that results in the attachment of a functional group or moiety. For example, a molecule may be functionalized by the introduction of a molecule that makes the molecule a strong nucleophile or strong electrophile.

The term “targeting moiety” refers to a moiety that localizes to or away from a specific locale. The moiety may be, for example, a protein, nucleic acid, nucleic acid analog, carbohydrate, or small molecule. The entity may be, for example, a therapeutic compound such as a small molecule, or a diagnostic entity such as a detectable label. The locale may be a tissue, a particular cell type or ligand, or a subcellular compartment. In one embodiment, the targeting moiety directs the localization of an agent. In preferred embodiment, the dendrimer composition selectively targets one or more types of immune cells such as activated microglia in the absence of an additional targeting moiety.

The term “prolonged residence time” refers to an increase in the time required for an agent to be cleared from a patient's body, or organ or tissue of that patient. In certain embodiments, “prolonged residence time” refers to an agent that is cleared with a half-life that is 10%, 20%, 50% or 75% longer than a standard of comparison such as a comparable agent without conjugation to a delivery vehicle such as a dendrimer. In certain embodiments, “prolonged residence time” refers to an agent that is cleared with a half-life of 2, 5, 10, 20, 50, 100, 200, 500, 1000, 2000, 5000, or 10000 times longer than a standard of comparison such as a comparable agent without a dendrimer that specifically target specific cell types.

The terms “incorporated” and “encapsulated” refer to incorporating, formulating, or otherwise including an agent into and/or onto a composition that allows for release, such as sustained release, of such agent in the desired application. The agent or other material can be incorporated into a dendrimer, by binding to one or more surface functional groups of such dendrimer (by covalent, ionic, or other binding interaction), by physical admixture, by enveloping the agent within the dendritic structure, and/or by encapsulating the agent inside the dendritic structure.

### II. Compositions

Analogs of the glutamine antagonist 6-diazo-5-oxo-L-norleucine (DON) have been developed. It has been established that systemically-administered hydroxyl-terminated PAMAM dendrimers can deliver one or more DON analogs to injured/activated target cell and/or tissues while minimizing peripheral exposure, and thus minimizing associated systemic toxicity including GI toxicity. Therefore, DON analogs conjugated to dendrimer nanoparticles suitable for delivery to one or more target immune cells in a subject are described.

Compositions of dendrimers complexed, covalently conjugated, or intra-molecularly dispersed or encapsulated with one or more 6-diazo-5-oxo-L-norleucine (DON) analogs having any of structures I-X or general Formulae A-C are provided. Exemplary dendrimers include generation 4, generation 5, generation 6, generation 7, or generation 8 dendrimers. In some embodiments the dendrimers are poly(amidoamine) (PAMAM) dendrimers, such as hydroxyl-terminated PAMAM dendrimers. In a preferred embodiment, the dendrimers are generation 4, generation 5, or generation 6, hydroxyl-terminated PAMAM dendrimers. In some embodiments, the dendrimers are covalently conjugated to one or more DON analogs, optionally via a linker or spacer moiety.

In certain embodiments, the presently disclosed dendrimer compositions have the structure of Formula (I):

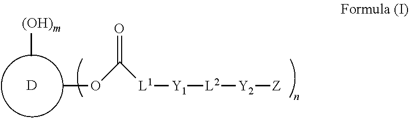

wherein, D is a dendrimer, preferably a G2 to G10 poly(amidoamine) (PAMAM) dendrimer;
L¹ and L² are each independently absent, C₁-C₁₂ alkylenyl, C₂-C₁₂ alkenylenyl, C₂-C₁₂ alkynylenyl, or (—CH₂CH₂O—)_{q} where q is 1-10;
Y₁ and Y₂ is are each independently absent, or a linker selected from secondary amides (—CONH—), tertiary amides (—CONR—), sulfonamide (—S(O)₂—NR—), secondary carbamates (—OCONH—; —NHCOO—), tertiary carbamates (—OCONR—; —NRCOO—), carbonate (—O—C(O)—O—), ureas (—NHCONH—; —NRCONH—; —NHCONR—, —NRCONR—), carbinols (—CHOH—, —CROH—), disulfide groups, hydrazones, hydrazides, and ethers (—O—), wherein R is an alkyl group, an aryl group, or a heterocyclic group;
Z is a DON analog of Formula (IIIa) or Formula (IIIb), or is selected from Structures I-X or general Formulae A-C;
n is 1 to 100; and m is 1 to 4000.

In some embodiments, the presently disclosed dendrimer compositions have the structure of Formula (Ia):

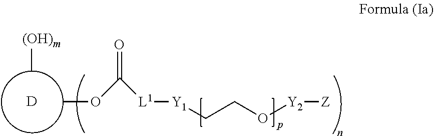

wherein, D is a dendrimer, preferably a G2 to G10 poly(amidoamine) (PAMAM) dendrimer;
L¹ is absent, C₁-C₁₂ alkylenyl, C₂-C₁₂ alkenylenyl, C₂-C₁₂ alkynylenyl, or (—CH₂CH₂O—)_{q} where q is 1-10;
Y₁ and Y₂ is are each independently absent, or a linker selected from secondary amides (—CONH—), tertiary amides (—CONR—), sulfonamide (—S(O)₂—NR—), secondary carbamates (—OCONH—; —NHCOO—), tertiary carbamates (—OCONR—; —NRCOO—), carbonate (—O—C(O)—O—), ureas (—NHCONH—; —NRCONH—; —NHCONR—, —NRCONR—), carbinols (—CHOH—, —CROH—), disulfide groups, hydrazones, hydrazides, and ethers (—O—), wherein R is an alkyl group, an aryl group, or a heterocyclic group;
Z is a DON analog of Formula (IIIa) or Formula (IIIb), or is selected from Structures I-X or general Formulae A-C;
p is 1 to 10, n is 1 to 100; and m is 1 to 4000.

In certain embodiments, the presently disclosed dendrimer compositions have the structure of Formula (II):

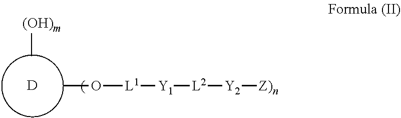

wherein, D is a dendrimer, preferably a G2 to G10 poly(amidoamine) (PAMAM) dendrimer;
L¹ and L² are each independently absent, C₁-C₁₂ alkylenyl, C₂-C₁₂ alkenylenyl, C₂-C₁₂ alkynylenyl, or (—CH₂CH₂O—)_{q} where q is 1-10;
Y₁ and Y₂ is are each independently absent, or a linker selected from secondary amides (—CONH—), tertiary amides (—CONR—), sulfonamide (—S(O)₂—NR—), secondary carbamates (—OCONH—; —NHCOO—), tertiary carbamates (—OCONR—; —NRCOO—), carbonate (—O—C(O)—O—), ureas (—NHCONH—; —NRCONH—; —NHCONR—, —NRCONR—), carbinols (—CHOH—, —CROH—), disulfide groups, hydrazones, hydrazides, and ethers (—O—), wherein R is an alkyl group, an aryl group, or a heterocyclic group;
Z is a DON analog of Formula (IIIa) or Formula (IIIb), or is selected from Structures I-X or general Formulae A-C;
n is 1 to 100; and m is 1 to 4000.

In some embodiments, the presently disclosed dendrimer compositions have the structure of Formula (IIa):

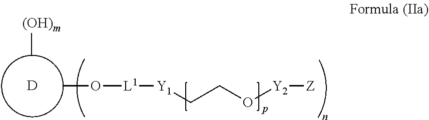

wherein, D is a dendrimer, preferably a G2 to G10 poly(amidoamine) (PAMAM) dendrimer;
L¹ is C₁-C₁₂ alkylenyl, C₂-C₁₂ alkenylenyl, C₂-C₁₂ alkynylenyl, or (—CH₂CH₂O—)_{q} where q is 1-10;
Y₁ and Y₂ is are each independently absent, or a linker selected from secondary amides (—CONH—), tertiary amides (—CONR—), sulfonamide (—S(O)₂—NR—), secondary carbamates (—OCONH—; —NHCOO—), tertiary carbamates (—OCONR—; —NRCOO—), carbonate (—O—C(O)—O—), ureas (—NHCONH—; —NRCONH—; —NHCONR—, —NRCONR—), carbinols (—CHOH—, —CROH—), disulfide groups, hydrazones, hydrazides, and ethers (—O—), wherein R is an alkyl group, an aryl group, or a heterocyclic group;
Z is a DON analog of Formula (IIIa) or Formula (IIIb), or is selected from Structures I-X or general Formulae A-C;
p is 1 to 10, n is 1 to 100; and m is 1 to 4000.

In certain embodiments of Formulae (I), (Ia), (II), and/or (IIa), Z is a DON analog of Formula (IIIa) or Formula (IIIb):

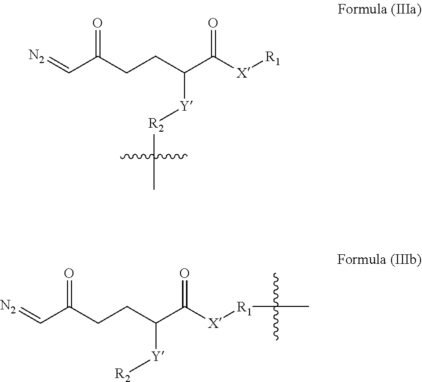

wherein X′ and Y′ are independently an oxygen or NH; and
wherein R₁ and R₂ are independently absent, a substituted or unsubstituted alkyl, a carbonyl, or a phosphoryl.

DON analog dendrimer conjugates are particularly suited for treating or preventing one or more symptoms associated with a variety of neurological, oncological, and immune disorders wherein dysregulated glutamine-dependent pathways and/or glutamate transmission are presumed pathogenic. Generally, one or more DON analogs is encapsulated, associated, and/or conjugated in the dendrimer complex at a concentration of about 0.01% to about 30%, preferably about 1% to about 20%, more preferably about 5% to about 20% by weight. Preferably, a DON analog is covalently conjugated to the dendrimer via one or more linkages such as disulfide, ester, ether, thioester, carbamate, carbonate, hydrazine, and amide, optionally via one or more spacers.

Dendrimers have the advantage that multiple therapeutic, prophylactic, and/or diagnostic agents can be delivered with the same dendrimers. One or more types of agents can be encapsulated, complexed or conjugated to the dendrimer. In one embodiment, the dendrimers are complexed with or conjugated to two or more different classes of agents, providing simultaneous delivery with different or independent release kinetics at the target site. In another embodiment, the dendrimers are covalently linked to at least one detectable moiety and at least one class of agents. In a further embodiment, dendrimer complexes each carrying different classes of agents are administered simultaneously for a combination treatment. Additional agents to be included in the particles to be delivered can be proteins or peptides, sugars or carbohydrate, nucleic acids or oligonucleotides, lipids, small molecules (e.g., molecular weight less than 2000 Dalton, preferably less than 1500 Dalton, more preferably 300-700 Dalton), or combinations thereof. The nucleic acid can be an oligonucleotide encoding a protein, for example, a DNA expression cassette or an mRNA. Representative oligonucleotides include siRNAs, microRNAs, DNA, and RNA. In some embodiments, the agent is a therapeutic antibody. Exemplary additional active agents include anti-inflammatory drugs, chemotherapeutics, anti-seizure agents, vasodilators, and anti-infective agents.

The presence of the additional agents can affect the zeta-potential or the surface charge of the particle. In one embodiment, the zeta potential of the dendrimers is between −100 mV and 100 mV, between −50 mV and 50 mV, between −25 mV and 25 mV, between −20 mV and 20 mV, between −10 mV and 10 mV, between −10 mV and 5 mV, between −5 mV and 5 mV, or between −2 mV and 2 mV. In a preferred embodiment, the surface charge is neutral or near-neutral. The range above is inclusive of all values from −100 mV to 100 mV.

### A. Glutamine Antagonists

Glutamine antagonists that inhibit glutamine metabolism, preferably in activated microglia, are useful for the treatment of one or more of neurological, oncological, and/or immune disorders wherein dysregulated glutamine-dependent pathways and/or glutamate transmission are presumed to be pathogenic or contributing to pathogenicity.

It has been discovered that systemically administered hydroxyl-terminated PAMAM dendrimers selectively can deliver glutamine antagonists to injured/activated tissues where they are retained, for at least two weeks, but are rapidly cleared from the periphery (plasma t½ is approximately 6-24 h). Thus, to enhance delivery of glutamine antagonists to activated target tissues while minimizing peripheral exposure and GI toxicity, one or more glutamine antagonists are preferably conjugated to dendrimers, preferably PAMAM dendrimers, for selective delivery to injured/activated tissues. In preferred embodiments, the glutamine antagonists are analogs of 6-diazo-5-oxo-L-norleucine.

1. Analogs of 6-diazo-5-oxo-L-norleucine (DON)

Analogs of 6-diazo-5-oxo-L-norleucine (DON) have been developed as glutamine antagonists, to inhibit glutamine metabolism in a subject in need thereof, preferably with less toxicity than DON.

Many tumors become largely dependent on glutamine to provide carbon and nitrogen building blocks needed for proliferation. Tumor glutamine dependence has been targeted with selective glutaminase inhibitors with some success. Several allosteric inhibitors including BPTES (bis-2-(5-phenylacetamido-1,2,4-thiadiazol-2-yl)ethyl sulfide), compound 968, and CB-839 (Calithera) have shown robust activity in cell culture experiments and promising single agent preclinical activity (Elgogary A, et al., Proc Natl Acad Sci USA 2016; 113(36):E5328-3611; Wang J B, et al., Cancer Cell 2010; 18(3):207-19; Gao M, et al., Mol Cell 2015; 59(2):298-308). CB-839 has proceeded into clinical studies. Although target engagement was clearly observed (Harding J J, et al., Journal of Clinical Oncology 2015; 33(15_suppl):2512), single agent antitumor activity was minimal. Combination trials are now underway with promising initial results (Meric-Bernstam F, et al., Journal of Clinical Oncology 2016; 34(15_suppl):4568; DeMichele A, et al., Journal of Clinical Oncology 2016; 34(15_suppl):1011).

6-diazo-5-oxo-norleucine (DON) is the best-studied broadly active glutamine antagonist, having multiple supporting biochemical, preclinical and clinical evaluations. DON was originally isolated from fermentation broth of a Streptomyces in the 1950s. DON inhibits glutamine-utilizing enzymes including glutaminase at low micromolar levels as well as multiple glutamine amidotransferases involved in de novo purine and pyrimidine synthesis, coenzyme synthesis, amino acid synthesis, and hexosamine production. Clinical studies of DON in the 1950s using low daily doses suggested antitumor activity, but later phase I and II trials of DON given intermittently at high doses were hampered by dose-limiting nausea and vomiting. Further clinical development of DON was abandoned.

Given the enormous therapeutic potential of glutamine antagonism, several DON analogs have been synthesized.

In some other instances, DON analogs may be used which have one of the following three general chemical formulae, wherein “AA” refers to an amino acid:

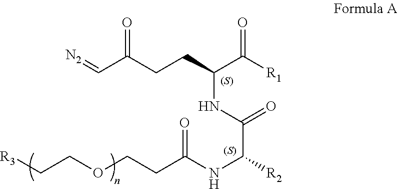

where R₁ is selected from —OCH₂CH₃, —OCH(CH₃)₂—OC(CH₃) 3, —NH₂,
   —NHCH₃, —NH-AA-COOCH(CH₃)₂, and —NH-AA-COOC(CH₃)₃;
where R₂ is selected from —H, —CH₃, —CH₂phenyl, —CH₂CH(CH₃)₂, —CH₂(indol-3-yl), and —CH₂CH₂C(═O)CH═N—N;
where R₃ is selected from —NH₂ and —OH; and
n is an integer value from 1 to 32,

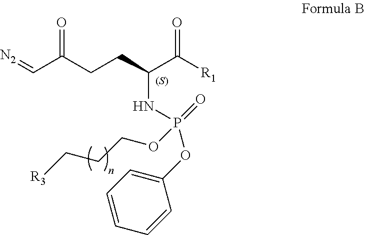

where R₁ is selected from —OCH₂CH₃, —OCH(CH₃)₂—OC(CH₃)₃, —NH₂,
   —NHCH₃, —NH-AA-COOCH(CH₃)₂, and —NH-AA-COOC(CH₃)₃;
where R₃ is selected from —NH₂ and —OH; and
n is an integer value from 1 to 16,

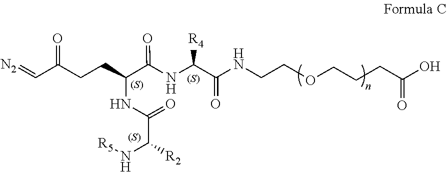

where R₂ is selected from —H, —CH₃, —CH₂phenyl, —CH₂CH(CH₃)₂, —CH₂(indol-3-yl), or —CH₂CH₂C(═O)CH═N≡N;
where R₄ is selected from —H, —CH₃, —CH₂phenyl, or —CH₂CH(CH₃)₂;
where R₅ is selected from —H, —Ac, —Fmoc, dimethylglycine, Ac-AA-C(═O)—, dimethylglycine-NH-AA-C(═O)—, dimethylglycine-NH-DON-C(═O)—, or C₈H₁₇O—C(═O)CH(NH₂)CH₂CH₂C(═O)—NH—6-diazo-5-oxo-L-norleucine —C(═O)—; and
n is an integer value from 1 to 32.

Preferred DON analogs are as shown in Structures I-X, below:

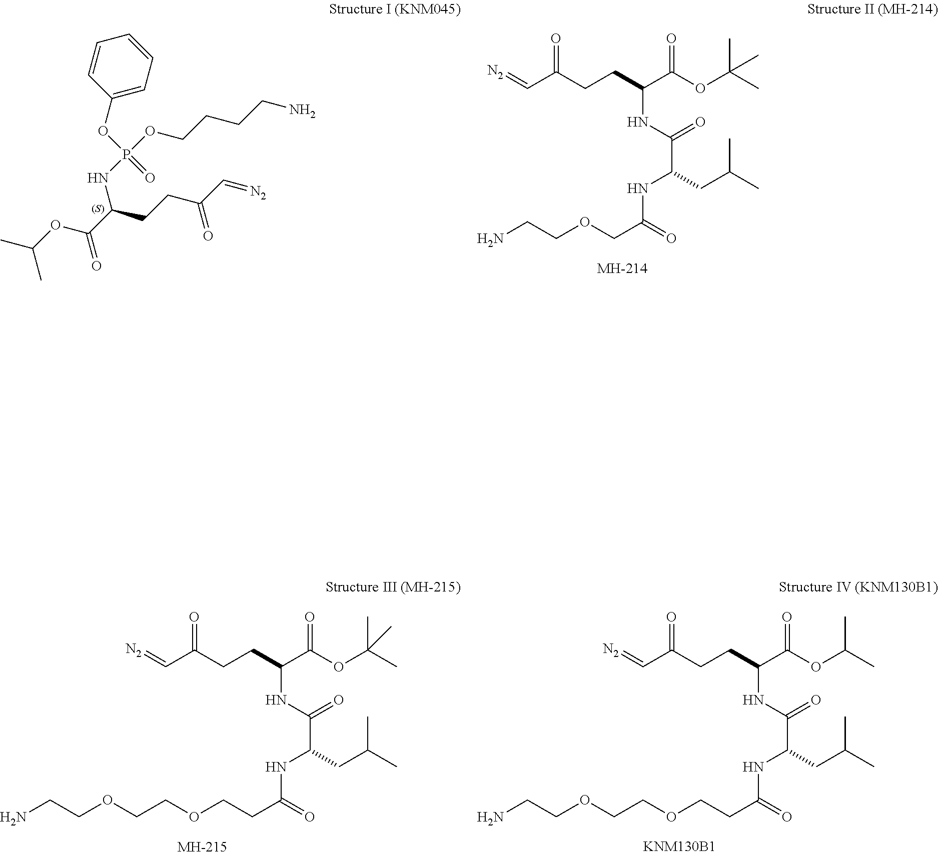

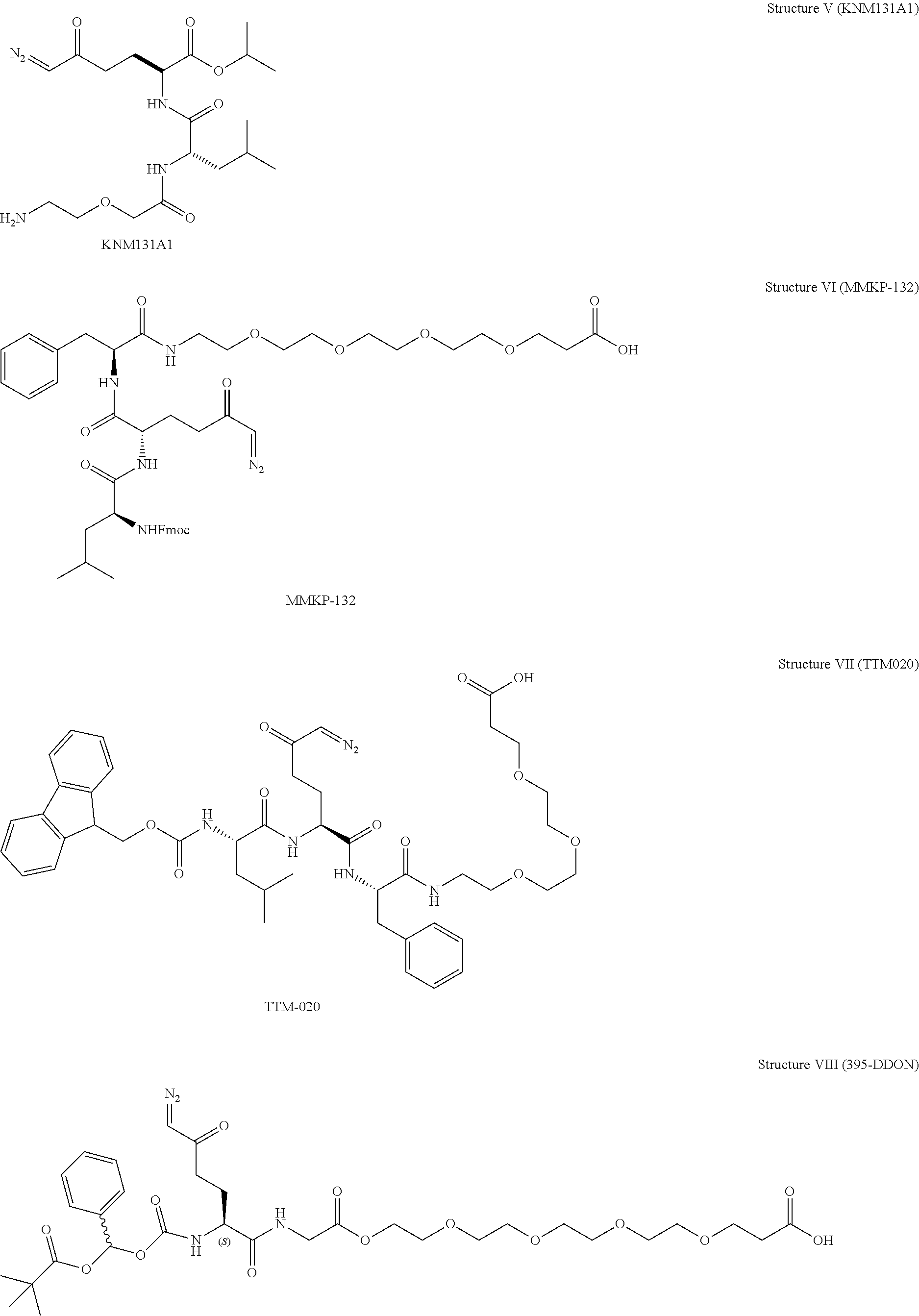

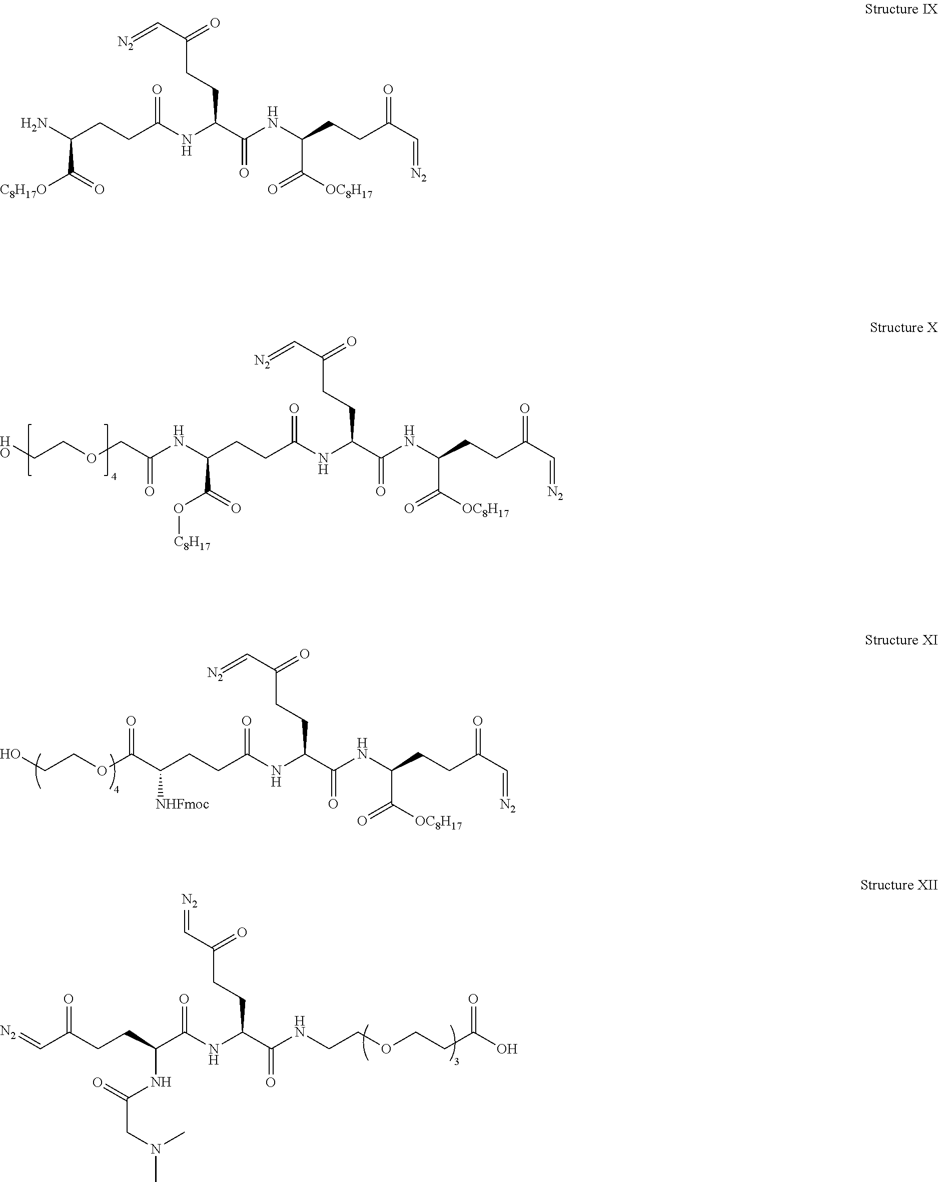

### B. Dendrimers

Dendrimer-glutamine antagonists include a dendrimer complexed, covalently conjugated, or intra-molecularly dispersed or encapsulated with one or more 6-diazo-5-oxo-L-norleucine (DON) analogs having a structure of any of structures I-X or general Formulae A-C.

Dendrimers are three-dimensional, hyperbranched, monodispersed, globular and polyvalent macromolecules including a high density of surface end groups (Tomalia, D. A., et al., Biochemical Society Transactions, 35, 61 (2007); and Sharma, A., et al., ACS Macro Letters, 3, 1079 (2014)). Due to their unique structural and physical features, dendrimers are useful as nanocarriers for various biomedical applications including targeted drug/gene delivery, imaging and diagnosis (Sharma, A., et al., RSC Advances, 4, 19242 (2014); Caminade, A. -M., et al., Journal of Materials Chemistry B, 2, 4055 (2014); Esfand, R., et al., Drug Discovery Today, 6, 427 (2001); and Kannan, R. M., et al., Journal of Internal Medicine, 276, 579 (2014)).

Dendrimer surface groups have a significant impact on their biodistribution (Nance, E., et al., Biomaterials, 101, 96 (2016)). Hydroxyl terminated generation 4 PAMAM dendrimers (approximately 4 nm size) without any targeting ligand cross the impaired BBB upon systemic administration in a rabbit model of cerebral palsy (CP) significantly more (>20 fold) as compared to healthy controls, and selectively target activated microglia and astrocytes (Lesniak, W. G., et al., Mol Pharm, 10 (2013)). The term “dendrimer” includes, but is not limited to, a molecular architecture with an interior core and layers (or “generations”) of repeating units which are attached to and extend from this interior core, each layer having one or more branching points, and an exterior surface of terminal groups attached to the outermost generation. In some embodiments, dendrimers have regular dendrimeric or “starburst” molecular structures.

Generally, dendrimers have a diameter between about 1 nm and about 50 nm, more preferably between about 1 nm and about 20 nm, between about 1 nm and about 10 nm, or between about 1 nm and about 5 nm. Conjugates are generally in the same size range, although large proteins such as antibodies may increase the size by 5-15 nm. In general, agent is encapsulated in a ratio of agent to dendrimer of between 1:1 and 4:1 for the larger generation dendrimers, i.e., four or higher. In preferred embodiments, the dendrimers have a diameter effective to penetrate brain tissue and to be retained in target cells for a prolonged period of time.

In some embodiments, dendrimers have a molecular weight between about 500 Daltons and about 100,000 Daltons, preferably between about 500 Daltons and about 50,000 Daltons, most preferably between about 1,000 Daltons and about 20,000 Dalton.

Suitable dendrimers scaffolds that can be used include poly(amidoamine), also known as PAMAM, or STARBURST™ dendrimers; polypropylamine (POPAM), polyethylenimine, polylysine, polyester, iptycene, aliphatic poly(ether), and/or aromatic polyether dendrimers. The dendrimers can have carboxylic, amine and/or hydroxyl terminations. In preferred embodiments, the dendrimers have hydroxyl terminations. Each dendrimer of the dendrimer complex may be same or of similar or different chemical nature than the other dendrimers (e.g., the first dendrimer may include a PAMAM dendrimer, while the second dendrimer may be a POPAM dendrimer).

The term “PAMAM dendrimer” means poly(amidoamine) dendrimer, which may contain different cores, with amidoamine building blocks, and can have carboxylic, amine and hydroxyl terminations of any generation including, but not limited to, generation 1 PAMAM dendrimers, generation 2 PAMAM dendrimers, generation 3 PAMAM dendrimers, generation 4 PAMAM dendrimers, generation 5 PAMAM dendrimers, generation 6 PAMAM dendrimers, generation 7 PAMAM dendrimers, generation 8 PAMAM dendrimers, generation 9 PAMAM dendrimers, or generation 10 PAMAM dendrimers. In the preferred embodiment, the dendrimers are soluble in the formulation and are generation (“G”) 4, 5 or 6 dendrimers. The dendrimers may have hydroxyl groups attached to their functional surface groups.

Methods for making dendrimers are known to those of skill in the art and generally involve a two-step iterative reaction sequence that produces concentric shells (generations) of dendritic 0-alanine units around a central initiator core (e.g., ethylenediamine-cores). Each subsequent growth step represents a new “generation” of polymer with a larger molecular diameter, twice the number of reactive surface sites, and approximately double the molecular weight of the preceding generation. Dendrimer scaffolds suitable for use are commercially available in a variety of generations. Preferable, the dendrimer compositions are based on generation 0, 1, 2, 3, 4, 5, 6, 7, 8, 9, or 10 dendrimeric scaffolds. Such scaffolds have, respectively, 4, 8, 16, 32, 64, 128, 256, 512, 1024, 2048, and 4096 reactive sites. Thus, the dendrimeric compounds based on these scaffolds can have up to the corresponding number of combined targeting moieties, if any, and agents.

In some embodiments, the dendrimers include a plurality of hydroxyl groups. Some exemplary high-density hydroxyl groups-containing dendrimers include commercially available polyester dendritic polymer such as hyperbranched 2,2-Bis(hydroxyl-methyl)propionic acid polyester polymer (for example, hyperbranched bis-MPA polyester-64-hydroxyl, generation 4), dendritic polyglycerols.

In some embodiments, the high-density hydroxyl groups-containing dendrimers are oligo ethylene glycol (OEG)-like dendrimers. For example, a generation 2 OEG dendrimer (D2-OH-60) can be synthesized using highly efficient, robust and atom economical chemical reactions such as Cu (I) catalyzed alkyne-azide click and photo catalyzed thiol-ene click chemistry.

Highly dense polyol dendrimer at very low generation in minimum reaction steps can be achieved by using an orthogonal hypermonomer and hypercore strategy, for example as described in WO2019094952. In some embodiments, the dendrimer backbone has non-cleavable polyether bonds throughout the structure to avoid the disintegration of dendrimer in vivo and to allow the elimination of such dendrimers as a single entity from the body (non-biodegradable).

In some embodiments, the dendrimer specifically targets a particular tissue region and/or cell type, preferably activated macrophages in the CNS. In preferred embodiments, the dendrimer specifically targets a particular tissue region and/or cell type without a targeting moiety.

In preferred embodiments, the dendrimers have a plurality of hydroxyl (—OH) groups on the periphery of the dendrimers. The preferred surface density of hydroxyl (—OH) groups is at least 1 OH group/nm² (number of hydroxyl surface groups/surface area in nm²). For example, in some embodiments, the surface density of hydroxyl groups is more than 2, 3, 4, 5, 6, 7, 8, 9, 10; preferably at least 10, 15, 20, 25, 30, 35, 40, 45, 50, or more than 50. In further embodiments, the surface density of hydroxyl (—OH) groups is between about 1 and about 50, preferably 5-20 OH group/nm² (number of hydroxyl surface groups/surface area in nm²) while having a molecular weight of between about 500 Da and about 10 kDa.

In some embodiments, the dendrimers may have a fraction of the hydroxyl groups exposed on the outer surface, with the others in the interior core of the dendrimers. In preferred embodiments, the dendrimers have a volumetric density of hydroxyl (—OH) groups of at least 1 OH group/nm³ (number of hydroxyl groups/volume in nm³). For example, in some embodiments, the volumetric density of hydroxyl groups is 2, 3, 4, 5, 6, 7, 8, 9, 10, or more than 10, 15, 20, 25, 30, 35, 40, 45, and 50. In some embodiments, the volumetric density of hydroxyl groups is between about 4 and about 50 groups/nm³, preferably between about 5 and about 30 groups/nm³, more preferably between about 10 and about 20 groups/nm³.

### C. Coupling Agents and Spacers

Dendrimer complexes can be formed of glutamine antagonists, optionally with one or more additional therapeutic agents or compounds conjugated or attached to a dendrimer, a dendritic polymer or a hyperbranched polymer. Optionally, the agents are conjugated to the dendrimers via one or more spacers/linkers via different linkages such as disulfide, ester, carbonate, carbamate, thioester, hydrazine, hydrazides, and amide linkages. The one or more spacers/linkers between a dendrimer and an agent can be designed to provide a releasable or non-releasable form of the dendrimer-active complexes in vivo. In some embodiments, the attachment occurs via an appropriate spacer that provides an ester bond between the agent and the dendrimer. In some embodiments, the attachment occurs via an appropriate spacer that provides an amide bond between the agent and the dendrimer. In preferred embodiments, one or more spacers/linkers between a dendrimer and an agent are added to achieve desired and effective release kinetics in vivo.

The term “spacers” includes compositions used for linking a therapeutically agent to the dendrimer. The spacer can be either a single chemical entity or two or more chemical entities linked together to bridge the polymer and the therapeutic agent or imaging agent. The spacers can include any small chemical entity, peptide or polymers having sulfhydryl, thiopyridine, succinimidyl, maleimide, vinylsulfone, and carbonate terminations.

The spacer can be chosen from among a class of compounds terminating in sulfhydryl, thiopyridine, succinimidyl, maleimide, vinylsulfone and carbonate group. The spacer can include thiopyridine terminated compounds such as dithiodipyridine, N-Succinimidyl 3-(2-pyridyldithio)-propionate (SPDP), Succinimidyl 6-(3-[2-pyridyldithio]-propionamido)hexanoate LC-SPDP or Sulfo-LC-SPDP. The spacer can also include peptides wherein the peptides are linear or cyclic essentially having sulfhydryl groups such as glutathione, homocysteine, cysteine and its derivatives, arg-gly-asp-cys (RGDC), cyclo(Arg-Gly-Asp-d-Phe-Cys) (c(RGDfC)), cyclo(Arg-Gly-Asp-D-Tyr-Cys), cyclo(Arg-Ala-Asp-d-Tyr-Cys). The spacer can be a mercapto acid derivative such as 3 mercapto propionic acid, mercapto acetic acid, 4 mercapto butyric acid, thiolan-2-one, 6 mercaptohexanoic acid, 5 mercapto valeric acid and other mercapto derivatives such as 2 mercaptoethanol and 2 mercaptoethylamine. The spacer can be thiosalicylic acid and its derivatives, (4-succinimidyloxycarbonyl-methyl-alpha-2-pyridylthio)toluene, (3-[2-pyridithio]propionyl hydrazide, The spacer can have maleimide terminations wherein the spacer includes polymer or small chemical entity such as bis-maleimido diethylene glycol and bis-maleimido triethylene glycol, Bis-Maleimidoethane, bismaleimidohexane. The spacer can include vinylsulfone such as 1,6-Hexane-bis-vinylsulfone. The spacer can include thioglycosides such as thioglucose. The spacer can be reduced proteins such as bovine serum albumin and human serum albumin, any thiol terminated compound capable of forming disulfide bonds. The spacer can include polyethylene glycol having maleimide, succinimidyl and thiol terminations.

The glutamine antagonists, or additional agent and/or targeting moiety can be either covalently attached or intra-molecularly dispersed or encapsulated. The dendrimer is preferably a PAMAM dendrimer up to generation 10, having carboxylic, hydroxyl, or amine terminations. In preferred embodiments, the dendrimer is linked to agents via a spacer ending in disulfide, ester or amide bonds.

### D. Additional Agents to be Delivered

Dendrimer-DON analog complexes can be used to deliver one or more additional active agents, particularly one or more agents to prevent or treat one or more symptoms of a disease or disorder. Suitable therapeutic, diagnostic, and/or prophylactic agents can be a biomolecule, such as peptides, proteins, carbohydrates, nucleotides or oligonucleotides, or a small molecule agent (e.g., molecular weight less than 2000 amu, preferably less than 1500 amu), including organic, inorganic, and organometallic agents. The agent can be encapsulated within the dendrimers, dispersed within the dendrimers, and/or associated with the surface of the dendrimer, either covalently or non-covalently.

### 1. Therapeutic and Prophylactic Agents

In some embodiments, the dendrimer complexes include one or more therapeutic, prophylactic, or prognostic agents that are complexed or conjugated to the dendrimers. Representative therapeutic agents include, but are not limited to, neuroprotective agents, anti-inflammatory agents, antioxidants, anti-infectious agents, and combinations thereof.

In one embodiment, the additional therapeutic agent is a steroid. Suitable steroids include biologically active forms of vitamin D3 and D2, such as those described in U.S. Pat. Nos. 4,897,388 and 5,939,407. The steroids may be co-administered to further aid in neurogenic stimulation or induction and/or prevention of neural loss, particularly for treatments of Alzheimer's disease. Estrogen and estrogen related molecules such as allopregnanolone can be co-administered with the neuro-enhancing agents to enhance neuroprotection as described in Brinton (2001) Learning and Memory 8 (3): 121-133.

Other neuroactive steroids, such as various forms of dehydroepi-androsterone (DHEA) as described in U.S. Pat. No. 6,552,010, can also be co-administered to further aid in neurogenic stimulation or induction and/or prevention of neural loss. Other agents that cause neural growth and outgrowth of neural networks, such as Nerve Growth Factor (NGF) and Brain-derived Neurotrophic Factor (BDNF), can be administered either simultaneously with or before or after the administration of THP. Additionally, inhibitors of neural apoptosis, such as inhibitors of calpains and caspases and other cell death mechanisms, such as necrosis, can be co-administered with the neuro-enhancing agents to further prevent neural loss associated with certain neurological diseases and neurological defects.

Representative small molecules include steroids such as methyl prednisone, dexamethasone, non-steroidal anti-inflammatory agents, including COX-2 inhibitors, corticosteroid anti-inflammatory agents, gold compound anti-inflammatory agents, immunosuppressive, anti-inflammatory and anti-angiogenic agents, anti-excitotoxic agents such as valproic acid, D-aminophosphonovalerate, D-aminophosphonoheptanoate, inhibitors of glutamate formation/release, baclofen, NMDA receptor antagonists, salicylate anti-inflammatory agents, ranibizumab, anti-VEGF agents, including aflibercept, and rapamycin. Other anti-inflammatory drugs include nonsteroidal drug such as indomethacin, aspirin, acetaminophen, diclofenac sodium and ibuprofen. The corticosteroids can be fluocinolone acetonide and methylprednisolone.

Representative oligonucleotides include siRNAs, microRNAs, DNA, and RNA.

### 2. Diagnostic Agents

In some cases, the additional agent is a diagnostic agent. Examples of diagnostic agents include paramagnetic molecules, fluorescent compounds, magnetic molecules, and radionuclides, x-ray imaging agents, and contrast media. Examples of other suitable contrast agents include gases or gas emitting compounds, which are radiopaque. Dendrimer complexes can further include agents useful for determining the location of administered compositions. Agents useful for this purpose include fluorescent tags, radionuclides and contrast agents.

Exemplary diagnostic agents include dyes, fluorescent dyes, near infra-red dyes, SPECT imaging agents, PET imaging agents and radioisotopes. Representative dyes include carbocyanine, indocarbocyanine, oxacarbocyanine, thüicarbocyanine and merocyanine, polymethine, coumarine, rhodamine, xanthene, fluorescein, boron-dipyrromethane (BODIPY), Cy5, Cy5.5, Cy7, VivoTag-680, VivoTag-S680, VivoTag-S750, AlexaFluor660, AlexaFluor680, AlexaFluor700, AlexaFluor750, AlexaFluor790, Dy677, Dy676, Dy682, Dy752, Dy780, DyLight547, Dylight647, HiLyte Fluor 647, HiLyte Fluor 680, HiLyte Fluor 750, IRDye 800CW, IRDye 800RS, IRDye 700DX, ADS780WS, ADS830WS, and ADS832WS.

Exemplary SPECT or PET imaging agents include chelators such as di-ethylene tri-amine penta-acetic acid (DTPA), 1,4,7,10-tetra-azacyclododecane-1,4,7,10-tetraacetic acid (DOTA), di-amine dithiols, activated mercaptoacetyl-glycyl-glycyl-gylcine (MAG3), and hydrazidonicotinamide (HYNIC).

Exemplary isotopes include Tc-94m, Tc-99m, In-111, Ga-67, Ga-68, Gd3+, Y-86, Y-90, Lu-177, Re-186, Re-188, Cu-64, Cu-67, Co-55, Co-57, F-18, Sc-47, Ac-225, Bi-213, Bi-212, Pb-212, Sm-153, Ho-166, and Dy-166.

In preferred embodiments, the dendrimer complex include one or more radioisotopes suitable for positron emission tomography (PET) imaging. Exemplary positron-emitting radioisotopes include carbon-11 (¹¹C), copper-64 (⁶⁴Cu), nitrogen-13 (¹³N), oxygen-15 (¹⁵O) gallium-68 (⁶⁸Ga), and fluorine-18 (¹⁸F), e.g., 2-deoxy-2-¹⁸F-fluoro-β-D-glucose (¹⁸F-FDG).

In further embodiments, a singular dendrimer complex composition can simultaneously treat and/or diagnose a disease or a condition at one or more locations in the body.

### III. Pharmaceutical Formulations

Pharmaceutical compositions including dendrimers and one or more 6-diazo-5-oxo-L-norleucine (DON) analogs are described. Pharmaceutical compositions including dendrimers and DON analogs may be formulated in a conventional manner using one or more physiologically acceptable carriers including excipients and auxiliaries which facilitate processing of the active compounds into preparations which can be used pharmaceutically. Proper formulation is dependent upon the route of administration chosen. In preferred embodiments, the compositions are formulated for parenteral delivery. In some embodiments, the compositions are formulated for intravenous injection. Typically, the compositions will be formulated in sterile saline or buffered solution for injection into the tissues or cells to be treated. The compositions can be stored lyophilized in single use vials for rehydration immediately before use. Other means for rehydration and administration are known to those skilled in the art.

In some embodiments, pharmaceutical formulations contain one or more dendrimer complexes in combination with one or more pharmaceutically acceptable excipients. Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials which are generally recognized as safe (GRAS), and may be administered to an individual without causing undesirable biological side effects or unwanted interactions.

Generally, pharmaceutically acceptable salts can be prepared by reaction of the free acid or base forms of an agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington's Pharmaceutical Sciences, 20th ed., Lippincott Williams & Wilkins, Baltimore, MD, 2000, p. 704. Examples of ophthalmic drugs sometimes administered in the form of a pharmaceutically acceptable salt include timolol maleate, brimonidine tartrate, and sodium diclofenac.

The compositions are preferably formulated in dosage unit form for ease of administration and uniformity of dosage. The phrase “dosage unit form” refers to a physically discrete unit of conjugate appropriate for the patient to be treated. It will be understood, however, that the total single administration of the compositions will be decided by the attending physician within the scope of sound medical judgment. The therapeutically effective dose can be estimated initially either in cell culture assays or in animal models, usually mice, rabbits, dogs, or pigs. The animal model is also used to achieve a desirable concentration range and route of administration. Such information should then be useful to determine useful doses and routes for administration in humans. Therapeutic efficacy and toxicity of conjugates can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index and it can be expressed as the ratio, LD50/ED50. Pharmaceutical compositions which exhibit large therapeutic indices are preferred. The data obtained from cell culture assays and animal studies can be used in formulating a range of dosages for human use.

In certain embodiments, the compositions are administered locally, for example, by injection directly into a site to be treated. In some embodiments, the compositions are injected, topically applied, or otherwise administered directly into the vasculature onto vascular tissue at or adjacent to a site of injury, surgery, or implantation. For example, in some embodiments, the compositions are topically applied to vascular tissue that is exposed, during a surgical procedure. Typically, local administration causes an increased localized concentration of the compositions which is greater than that which can be achieved by systemic administration.

Pharmaceutical compositions formulated for administration by parenteral (intramuscular, intraperitoneal, intravenous (IV) or subcutaneous injection) and enteral routes of administration are described.

### A. Parenteral Administration

In some embodiments, pharmaceutical compositions including dendrimers and DON analogs are formulated for parenteral administration. The phrases “parenteral administration” and “administered parenterally” are art-recognized terms, and include modes of administration other than enteral and topical administration, such as injections via intravenous (i.v.), intramuscular (i.m.), intrapleural, intravascular, intrapericardial, intraarterial, intrathecal, intracapsular, intraorbital, intracardiac, intradennal, intraperitoneal (i.p.), transtracheal, subcutaneous (s.c.), subcuticular, intraarticular, subcapsular, subarachnoid, intraspinal and intrastemal injection and infusion. The dendrimers also can be administered parenterally, for example, by subdural, intravenous, intrathecal, intraventricular, intraarterial, intra-amniotic, intraperitoneal, or subcutaneous routes.

For liquid formulations, pharmaceutically acceptable carriers may be, for example, aqueous or non-aqueous solutions, suspensions, emulsions or oils. Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media. The dendrimers can also be administered in an emulsion, for example, water in oil. Examples of oils are those of animal, vegetable, or synthetic origin, for example, peanut oil, soybean oil, mineral oil, olive oil, sunflower oil, fish-liver oil, sesame oil, cottonseed oil, corn oil, olive, and mineral oil. Suitable fatty acids for use in parenteral formulations include, for example, oleic acid, stearic acid, and isostearic acid. Ethyl oleate and isopropyl myristate are examples of suitable fatty acid esters.

Formulations suitable for parenteral administration can include antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Intravenous vehicles can include fluid and nutrient replenishers, electrolyte replenishers such as those based on Ringer's dextrose. In general, water, saline, aqueous dextrose and related sugar solutions, and glycols such as propylene glycols or polyethylene glycol are preferred liquid carriers, particularly for injectable solutions.

Injectable pharmaceutical carriers for injectable compositions are well-known to those of ordinary skill in the art (see, e.g., Pharmaceutics and Pharmacy Practice, J.B. Lippincott Company, Philadelphia, PA, Banker and Chalmers, eds., pages 238-250 (1982), and ASHP Handbook on Injectable Drugs, Trissel, 15th ed., pages 622-630 (2009)).

### B. Enteral Administration

In some embodiments, pharmaceutical compositions including dendrimers and DON analogs are formulated for enteral administration. The carriers or diluents useful for compositions that can be administered enterally may be solid carriers such as capsule or tablets or diluents for solid formulations, liquid carriers or diluents for liquid formulations, or mixtures thereof, and can include food mixtures and liquid feeding formulas.

For liquid formulations, pharmaceutically acceptable carriers may be, for example, aqueous or non-aqueous solutions, suspensions, emulsions or oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Vehicles can include, for example, fluid and nutrient replenishers, electrolyte replenishers such as those based on Ringer's dextrose. In general, water, saline, aqueous dextrose and related sugar solutions are preferred liquid carriers. These can also be formulated with proteins, fats, saccharides and other components of infant formulas.

In preferred embodiments, the compositions are formulated for oral administration. Oral formulations may be in the form of solutions or suspensions, chewing gum, gel strips, tablets, capsules or lozenges. Encapsulating substances for the preparation of enteric-coated oral formulations include cellulose acetate phthalate, polyvinyl acetate phthalate, hydroxypropyl methylcellulose phthalate and methacrylic acid ester copolymers. Solid oral formulations such as capsules or tablets are preferred. Elixirs and syrups also are well known oral formulations.

### IV. Methods of Making Dendrimers and Conjugates or Complexes Thereof

Methods of making one or more 6-diazo-5-oxo-L-norleucine (DON) analogs having any of structures I-X and preparing dendrimers complexed with one or more of these DON analogs are provided. Methods of making one or more 6-diazo-5-oxo-L-norleucine (DON) analogs having structures encompassed by general Formulae A-C, as discussed above, and preparing dendrimers complexed with one or more of these DON analogs can be accomplished by the same or similar synthetic methods used to make structures I-X and dendrimers thereof, as described below, which is well within the skills of the person of ordinary skill in the art.

A. Methods of Making Analogs of 6-diazo-5-oxo-L-norleucine

Exemplary synthesis methods of one or more 6-diazo-5-oxo-L-norleucine (DON) analogs having any of structures I-X are described in Example 1.

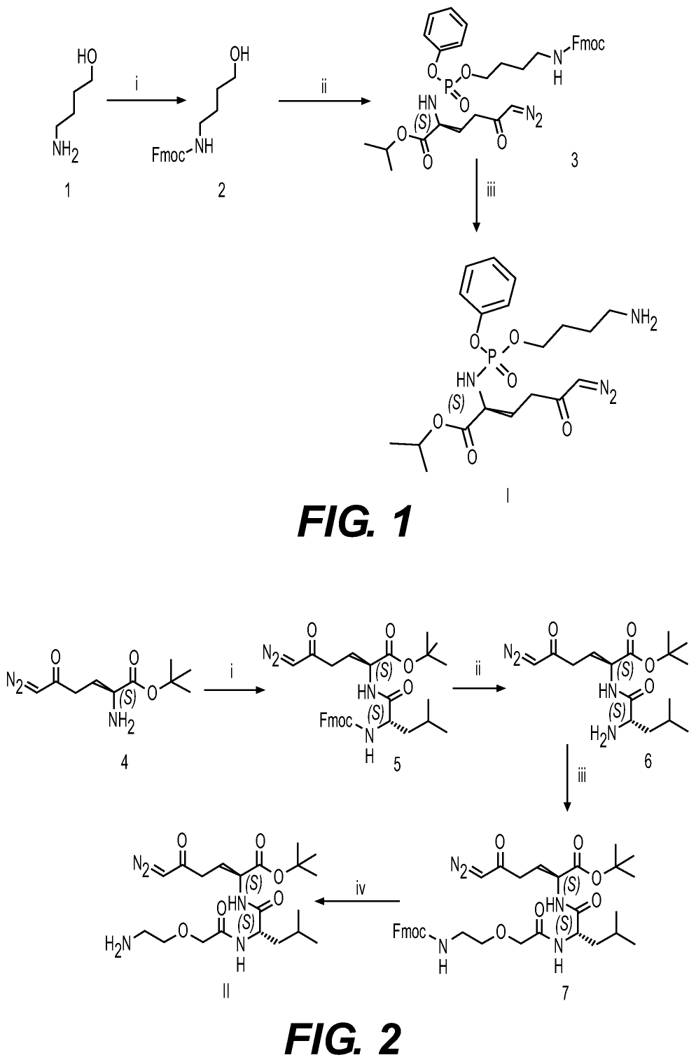

In some embodiments, the synthetic scheme for Structure I is as shown in FIG. 1 and exemplary reaction conditions include (i) Fmoc-Cl, NaHCO₃, dioxane, 0° C., 2 h, 67%; (ii) isopropyl (S)-2-amino-6-diazo-5-oxohexanoate, phenyl dichlorophosphate, 2,4,6-trimethylpyridine, THF, 0° C. to RT, 5 h, 41%; (iii) piperidine, DCM, RT, 2.5 h, 73%.

In some embodiments, the synthetic scheme for Structure II is as shown in FIG. 2 and exemplary reaction conditions include (i) HATU, DIEA, Fmoc-L-Leu-OH, DCM, 0° C. to RT, 20 h, 76%; (ii) piperidine, DCM, RT, 2 h, 93%; (iii) 5-(Fmoc-amino)-3-oxopentanoic acid, HATU, DIEA, DMF, 0° C. to RT, 1 h, 70%; (iv) piperidine, DCM, RT, 2 h, 93%.

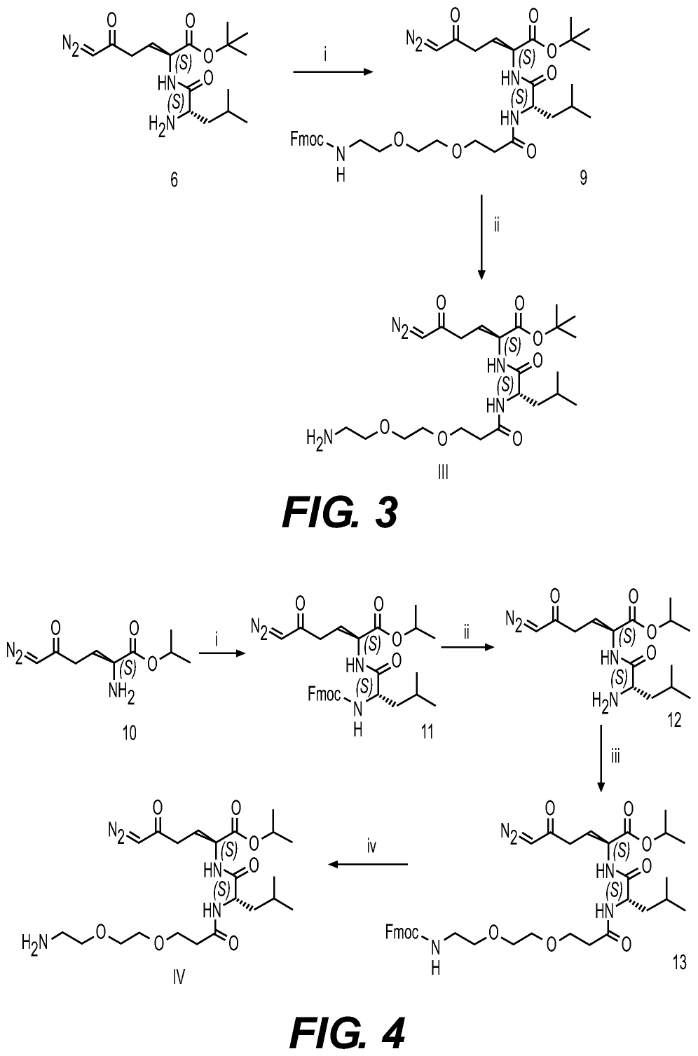

In some embodiments, the synthetic scheme for Structure III is as shown in FIG. 3 and exemplary reaction conditions include (i) Fmoc-9-amino-4,7-dioxanonanoic acid, HATU, DIEA, DMF, 0° C. to RT, 1 h, 67%; (ii) piperidine, DCM, RT, 3 h, 89%.

In some embodiments, the synthetic scheme for Structure IV is as shown in FIG. 4 and exemplary reaction conditions include (i) HATU, DIEA, Fmoc-L-Leu-OH, DCM, 0° C. to RT, 20 h, 96%; (ii) diethylamine, DCM, RT, 1.5 h, 88%; (iii) Fmoc-9-amino-4,7-dioxanonanoic acid, HATU, DIEA, DMF, 0° C. to RT, 1 h, 86%; (iv) piperidine, DCM, RT, 1 h, 87%.

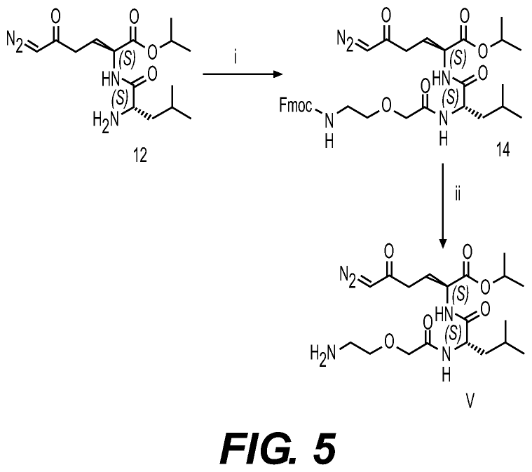

In some embodiments, the synthetic scheme for Structure V is as shown in FIG. 5 and exemplary reaction conditions include (i) 5-(Fmoc-amino)-3-oxopentanoic acid, HATU, DIEA, DMF, 0° C. to RT, 1 h, 89%; (ii) piperidine, DCM, RT, 1 h, 74%.

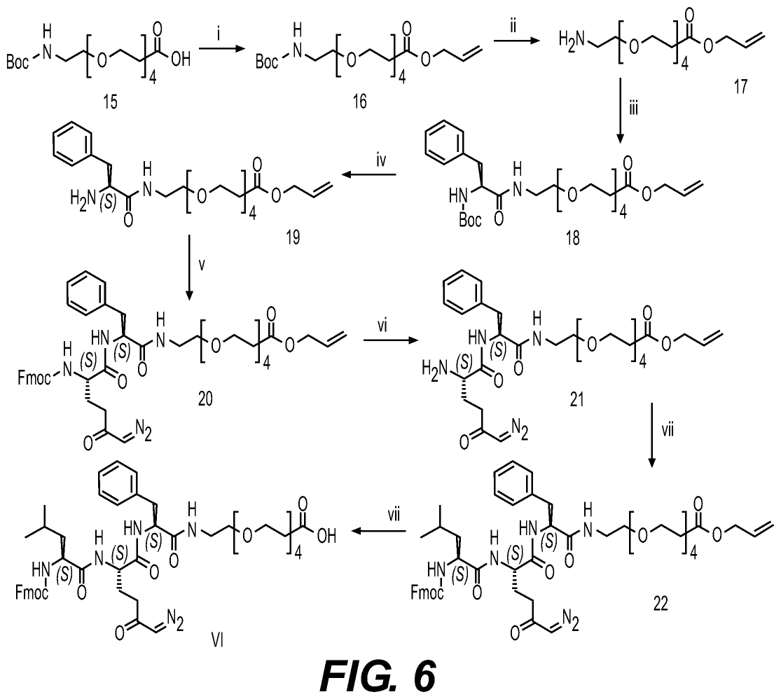

In some embodiments, the synthetic scheme for Structure VI is as shown in FIG. 6 and exemplary reaction conditions include (i) Allyl bromide, Cs2CO3, MeCN, RT, 1 h, 96%; (ii) TFA, DCM; (iii) Boc-L-Phe-OH, HATU, DIEA, DMF, RT, 3 h, 88%; (iv) TFA, DCM; (v) a DIEA, DCM; b (S)-2-(Fmoc-amino)-6-diazo-5-oxohexanoic acid, EDC, HOBt, DCM, RT, 2 h, 71%; (vi) diethylamine, DCM, 75%; (vii) Fmoc-L-Leu-OH, DIEA, HBTU, DCM, RT, 2 h, 47%; (viii) Pd(PPh3)4, dimedone, THF, 63%.

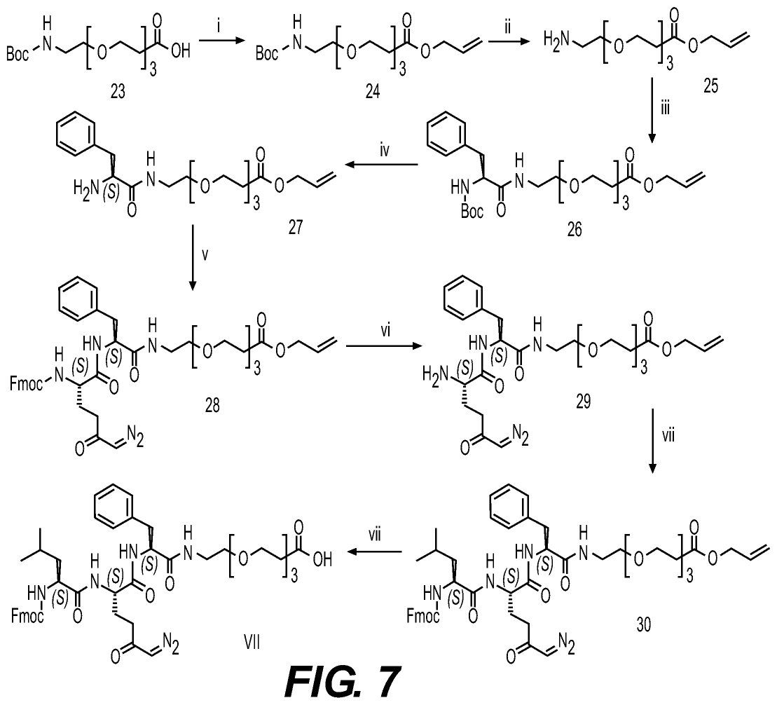

In some embodiments, the synthetic scheme for Structure VII is as shown in FIG. 7 and exemplary reaction conditions include (i) Allyl bromide, Cs₂CO₃, MeCN, RT, 1 h, 96%; (ii) TFA, DCM; (iii) Boc-L-Phe-OH, HATU, DIEA, DMF, RT, 3 h, 88%; (iv) TFA, DCM; (v) a DIEA, DCM; b (S)-2-(Fmoc-amino)-6-diazo-5-oxohexanoic acid, EDC, HOBt, DCM, RT, 2 h, 71%; (vi) diethylamine, DCM, 75%; (vii) Fmoc-L-Leu-OH, DIEA, HBTU, DCM, RT, 2 h, 47%; (viii) Pd(PPh3)4, dimedone, THF, 63%.

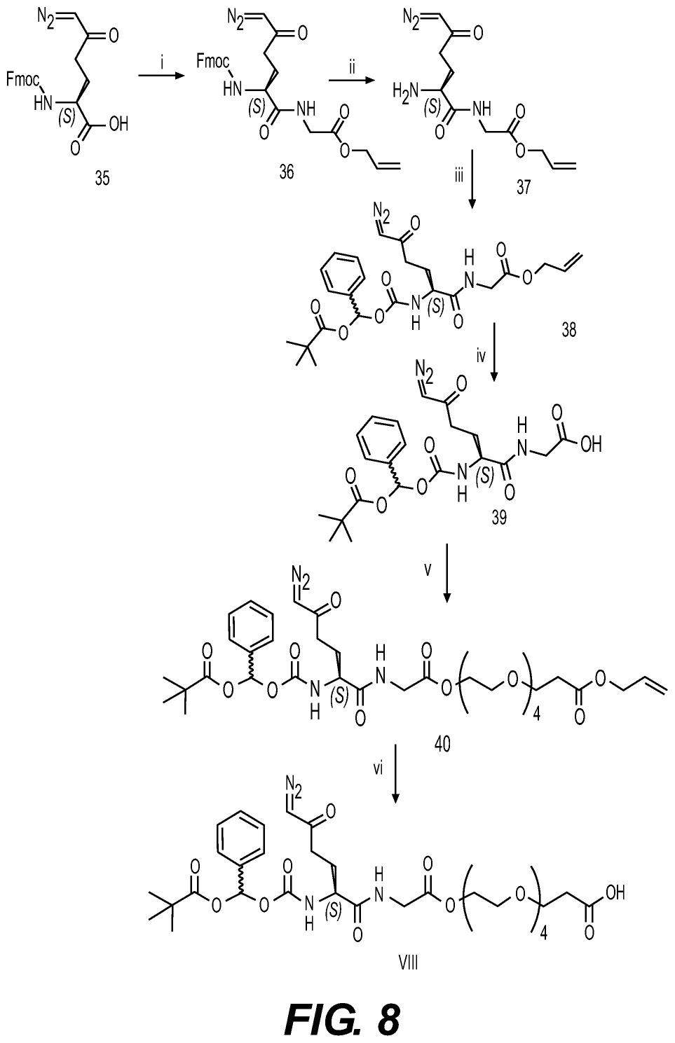

In some embodiments, the synthetic scheme for Structure VIII is as shown in FIG. 8 and exemplary reaction conditions include (i) HATU, DIEA, H-Gly-OAll*HCl, DCM, 0° C. to RT, 2.5 h, 77%; (ii) diethylamine, DCM, RT, 2.5 h; (iii) (((4-nitrophenoxy)carbonyl)oxy)(phenyl)methyl pivalate, DMF, RT, 4 h, 54% over 2 steps; (iv) Pd(PPh3)4, phenyl silane, DCM, RT, 0.5 h; (v) HATU, DIEA, H₂N-PEG5-COOAll*TFA salt, DMF, 0° C. to RT, 4 h; (vi) Pd(PPh3)4, phenyl silane, DCM, RT, 0.5 h.

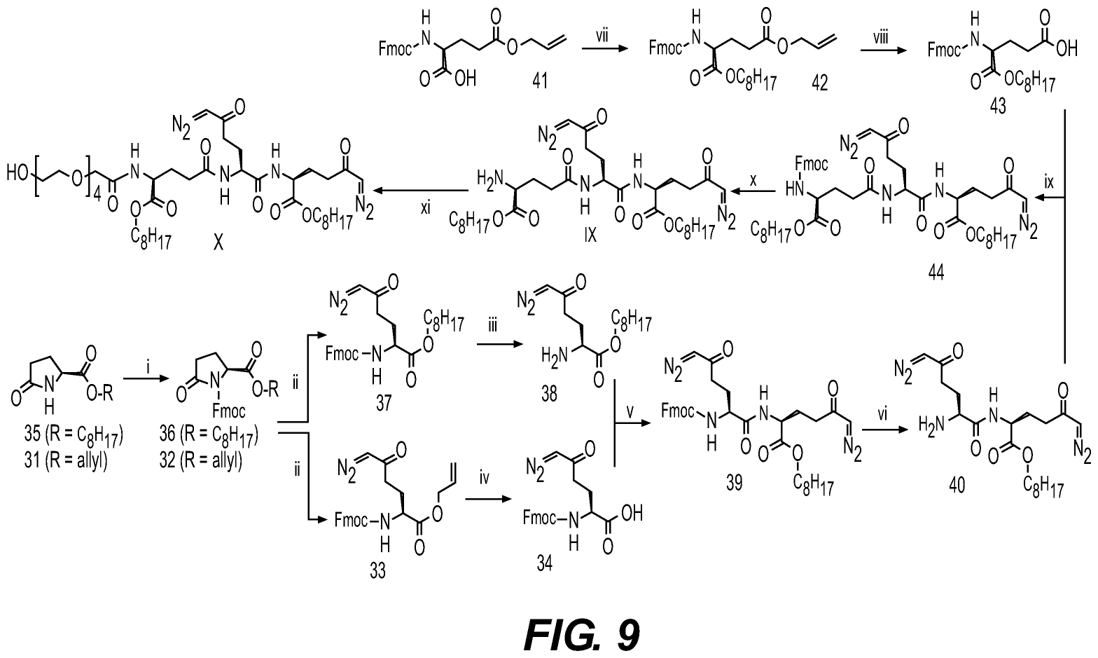

In some embodiments, the synthetic scheme for Structures IX and X is as shown in FIG. 9 and exemplary reaction conditions include (i) LiHMDS, Fmoc-Cl, THF, −78° C.; (ii) n-BuLi, TMSCHN₂, THF, −116 to −78° C.; (iii) piperidine, DMF, RT; (iv) Pd(PPh3)4, PhSiH₃, DCM, 0° C.; (v) HATU, DCM, DIEA, 0° C.; (vi) piperidine, DMF, RT; (vii) C₈H₁₇OH, DCC, DMAP, DCM, RT; (viii) Pd(PPh₃)₄, PhSiH₃, DCM, RT; (ix) HATU, DIEA, DCM, 0° C.; (x) piperidine, DMF, RT; (xi) HOBt, EDC, DIEA, THF, RT.

### B. Methods of Making Dendrimers

Dendrimers complexed with one or more DON analogs can be prepared via a variety of chemical reaction steps. Dendrimers are usually synthesized according to methods allowing controlling their structure at every stage of construction. The dendritic structures are mostly synthesized by two main different approaches: divergent or convergent.

In some embodiments, dendrimers are prepared using divergent methods, in which the dendrimer is assembled from a multifunctional core, which is extended outward by a series of reactions, commonly a Michael reaction. The strategy involves the coupling of monomeric molecules that possesses reactive and protective groups with the multifunctional core moiety, which leads to stepwise addition of generations around the core followed by removal of protecting groups. For example, PAMAM-NH₂ dendrimers are first synthesized by coupling N-(2-aminoethyl) acryl amide monomers to an ammonia core.

In other embodiments, dendrimers are prepared using convergent methods, in which dendrimers are built from small molecules that end up at the surface of the sphere, and reactions proceed inward, building inward, and are eventually attached to a core.

Many other synthetic pathways exist for the preparation of dendrimers, such as the orthogonal approach, accelerated approaches, the Double-stage convergent method or the hypercore approach, the hypermonomer method or the branched monomer approach, the Double exponential method; the Orthogonal coupling method or the two-step approach, the two monomers approach, AB₂-CD₂ approach.

In some embodiments, the core of the dendrimer, one or more branching units, one or more linkers/spacers, and/or one or more surface groups can be modified to allow conjugation to further functional groups (branching units, linkers/spacers, surface groups, etc.), monomers, and/or agents via click chemistry, employing one or more Copper-Assisted Azide-Alkyne Cycloaddition (CuAAC), Diels-Alder reaction, thiol-ene and thiol-yne reactions, and azide-alkyne reactions (Arseneault M et al., Molecules. 2015 May 20; 20(5):9263-94). In some embodiments, pre-made dendrons are clicked onto high-density hydroxyl polymers. ‘Click chemistry’ involves, for example, the coupling of two different moieties (e.g., a core group and a branching unit; or a branching unit and a surface group) via a 1,3-dipolar cycloaddition reaction between an alkyne moiety (or equivalent thereof) on the surface of the first moiety and an azide moiety (e.g., present on a triazine composition or equivalent thereof), or any active end group such as, for example, a primary amine end group, a hydroxyl end group, a carboxylic acid end group, a thiol end group, etc.) on the second moiety.

In some embodiments, dendrimer synthesis replies upon one or more reactions such as thiol-ene click reactions, thiol-yne click reactions, CuAAC, Diels-Alder click reactions, azide-alkyne click reactions, Michael Addition, epoxy opening, esterification, silane chemistry, and a combination thereof.

Any existing dendritic platforms can be used to make dendrimers of desired functionalities, i.e., with a high-density of surface hydroxyl groups by conjugating high-hydroxyl containing moieties such as 1-thio-glycerol or pentaerythritol. Exemplary dendritic platforms such as polyamidoamine (PAMAM), poly (propylene imine) (PPI), poly-L-lysine, melamine, poly (etherhydroxylamine) (PEHAM), poly (esteramine) (PEA) and polyglycerol can be synthesized and explored.

Dendrimers also can be prepared by combining two or more dendrons. Dendrons are wedge-shaped sections of dendrimers with reactive focal point functional groups. Many dendron scaffolds are commercially available. They come in 1, 2, 3, 4, 5, and 6th generations with, respectively, 2, 4, 8, 16, 32, and 64 reactive groups. In certain embodiments, one type of agents is linked to one type of dendron and a different type of agent is linked to another type of dendron. The two dendrons are then connected to form a dendrimer. The two dendrons can be linked via click chemistry i.e., a 1,3-dipolar cycloaddition reaction between an azide moiety on one dendron and alkyne moiety on another to form a triazole linker.

Exemplary methods of making dendrimers are described in detail in International Patent Publication Nos. WO2009/046446, WO2015168347, WO2016025745, WO2016025741, WO2019094952, and U.S. Pat. No. 8,889,101.

### C. Dendrimer-DON Analog Complexes

Dendrimer complexes can be formed of DON analogs conjugated or complexed to a dendrimer, a dendritic polymer or a hyperbranched polymer. Exemplary dendrimer-DON analog complexes are shown in Table 1 below.

The complexes can also include additional therapeutic, prophylactic or diagnostic agents. Conjugation of one or more agents to a dendrimer are known in the art and are described in detail in U.S. Published Application Nos. US 2011/0034422, US 2012/0003155, and US 2013/0136697.

In some embodiments, one or more agents are covalently attached to the dendrimers. In some embodiments, the agents are attached to the dendrimer via a linking moiety that is designed to be cleaved in vivo. The linking moiety can be designed to be cleaved hydrolytically, enzymatically, or combinations thereof, so as to provide for the sustained release of the agents in vivo. Both the composition of the linking moiety and its point of attachment to the agent, are selected so that cleavage of the linking moiety releases either an agent, or a suitable prodrug thereof. The composition of the linking moiety can also be selected in view of the desired release rate of the agents.

In some embodiments, the attachment occurs via one or more of disulfide, ester, ether, thioester, carbamate, carbonate, hydrazine, or amide linkages. In preferred embodiments, the attachment occurs via an appropriate spacer that provides an ester bond or an amide bond between the agent and the dendrimer depending on the desired release kinetics of the agent. In some cases, an ester bond is introduced for releasable form of agents. In other cases, an amide bond is introduced for non-releasable form of agents.

Linking moieties generally include one or more organic functional groups. Examples of suitable organic functional groups include secondary amides (—CONH—), tertiary amides (—CONR—), sulfonamide (—S(O)₂—NR—), secondary carbamates (—OCONH—; —NHCOO—), tertiary carbamates (—OCONR—; —NRCOO—), carbonate (—O—C(O)—O—), ureas (—NHCONH—; —NRCONH—; —NHCONR—, —NRCONR—), carbinols (—CHOH—, —CROH—), disulfide groups, hydrazones, hydrazides, ethers (—O—), and esters (—COO—, —CH₂O₂C—, CHRO₂C—), wherein R is an alkyl group, an aryl group, or a heterocyclic group. In general, the identity of the one or more organic functional groups within the linking moiety is chosen in view of the desired release rate of the agents. In addition, the one or more organic functional groups can be selected to facilitate the covalent attachment of the agents to the dendrimers. In preferred embodiments, the attachment can occur via an appropriate spacer that provides a disulfide bridge between the agent and the dendrimer. The dendrimer complexes are capable of rapid release of the agent in vivo by thiol exchange reactions, under the reduced conditions found in body.

In certain embodiments, the linking moiety includes one or more of the organic functional groups described above in combination with a spacer group. The spacer group can be composed of any assembly of atoms, including oligomeric and polymeric chains; however, the total number of atoms in the spacer group is preferably between 3 and 200 atoms, more preferably between 3 and 150 atoms, more preferably between 3 and 100 atoms, most preferably between 3 and 50 atoms. Examples of suitable spacer groups include alkyl groups, heteroalkyl groups, alkylaryl groups, oligo- and polyethylene glycol chains, and oligo- and poly(amino acid) chains. Variation of the spacer group provides additional control over the release of the agents in vivo. In embodiments where the linking moiety includes a spacer group, one or more organic functional groups will generally be used to connect the spacer group to both the anti-inflammatory agent and the dendrimers.

Reactions and strategies useful for the covalent attachment of agents to dendrimers are known in the art. See, for example, March, “Advanced Organic Chemistry,” 5th Edition, 2001, Wiley-Interscience Publication, New York) and Hermanson, “Bioconjugate Techniques,” 1996, Elsevier Academic Press, U.S.A. Appropriate methods for the covalent attachment of a given agent can be selected in view of the linking moiety desired, as well as the structure of the agents and dendrimers as a whole as it relates to compatibility of functional groups, protecting group strategies, and the presence of labile bonds.

The optimal drug loading will necessarily depend on many factors, including the choice of drug, dendrimer structure and size, and tissues to be treated. In some embodiments, the one or more agents are encapsulated, associated, and/or conjugated to the dendrimer at a concentration of about 0.01% to about 45%, preferably about 0.1% to about 30%, about 0.1% to about 20%, about 0.1% to about 10%, about 1% to about 10%, about 1% to about 5%, about 3% to about 20% by weight, and about 3% to about 10% by weight. However, optimal drug loading for any given drug, dendrimer, and site of target can be identified by routine methods, such as those described.

In some embodiments, conjugation of agents and/or linkers occurs through one or more surface and/or interior groups. Thus, in some embodiments, the conjugation of agents/linkers occurs via about 1%, 2%, 3%, 4%, or 5% of the total available surface functional groups, preferably hydroxyl groups, of the dendrimers prior to the conjugation. In other embodiments, the conjugation of agents/linkers occurs on less than 5%, less than 10%, less than 15%, less than 20%, less than 25%, less than 30%, less than 35%, less than 40%, less than 45%, less than 50%, less than 55%, less than 60%, less than 65%, less than 70%, less than 75% total available surface functional groups of the dendrimers prior to the conjugation. In preferred embodiments, dendrimer complexes retain an effective amount of surface functional groups for targeting to specific cell types, whilst conjugated to an effective amount of agents for treat, prevent, and/or image the disease or disorder.

### V. Methods of Use

Methods of using the dendrimer and DON analog complex compositions are described. In some embodiments, the dendrimer and DON analog complexes are used to treat cancer. In other embodiments, the dendrimer and DON analog complexes are used to treat CNS and immune disorders. The methods typically include administering to a subject in a need thereof an effective amount of a composition including dendrimer and one or more active agents to modulate the glutamine metabolism, for example, to increase anti-tumor response. The methods can be used for treating one or more conditions and/or diseases associated with dysregulated glutamine-dependent pathways and/or glutamate transmission. In some embodiments, the methods are used to effectively reduce tumor growth. The methods include administering an effective amount of a composition including dendrimer complexed with, conjugated to, or encapsulated with one or more analogs of 6-diazo-5-oxo-L-norleucine (DON) to a subject in need thereof. In preferred embodiments, the methods include administering an effective amount of a composition including dendrimer complexed with or conjugated to one or more DON analog as shown in Structures I-XII or analogs of general Formulae A-C, or pharmaceutically acceptable salt thereof to the subject.

### A. Methods of Treatment

The dendrimer and DON analog compositions and formulations thereof can be administered to treat disorders associated with infection, inflammation, and/or cancer, particularly those associated with dysregulated glutamine-dependent pathways and/or glutamate transmission, especially in the CNS. The compositions can also be used for treatment of other diseases, disorders and injury including proliferative diseases and treatment of other tissues where the nerves play a role in the disease or disorder. The compositions and methods are also suitable for prophylactic use.

Typically, an effective amount of dendrimer complexes including a combination of a dendrimer with one or more analogs of 6-diazo-5-oxo-L-norleucine (DON) are administered to an individual in need thereof. The dendrimers may also include a targeting agent, but as demonstrated by the examples, these are not required for delivery to injured or diseased tissue in the brain.

The amount of dendrimer and DON analog complexes administered to the subject is selected to deliver an effective amount to reduce, prevent, or otherwise alleviate one or more clinical or molecular symptoms of the disease or disorder to be treated compared to a control, for example, a subject treated with the active agent without dendrimer.

In some embodiments, dendrimer and DON analog complexes are administered to a subject in an amount effective to decrease glutaminase activity and/or reduce glutamate release in one or more target cells associated with the diseased or injured tissue of one or more of neurological, oncological, and immune disorders. In preferred embodiments, the composition is administered in an amount effective to decrease glutaminase activity and/or reduce glutamate release in activated microglia, especially those associated with the diseased or injured tissue of one or more of neurological, oncological, and immune disorders.

### B. Conditions to be Treated

The compositions of dendrimer and DON analog complexes are suitable for treating one or more of the neurological, oncological, and/or immune diseases, conditions, and disorders wherein dysregulated glutamine-dependent pathways and/or glutamate transmission are presumed pathogenic. In preferred embodiments, the one or more of the neurological, oncological, and immune diseases, conditions, and disorders are further associated with pathological activation of microglia and astrocytes. These include, but are not limited to, lung, colorectal, brain, and pancreatic cancers, depression, cognitive deficit, ischemia, neuroinflammation, Alzheimer's disease, Multiple Sclerosis, tuberculosis, Lupus, rheumatoid arthritis, and viral infections.

Microglia are a type of neuroglia (glial cell) located throughout the brain and spinal cord. Microglia account for 10-15% of all cells found within the brain. Microglia are involved in the primary response to microorganisms, neuroinflammation, homeostasis, and tissue regeneration, as well as contributing to the pathogenesis of neurodegenerative diseases. Research has shown that microglial diversity, multi-functionality, and their relationship with glutamate are crucial to determining their roles in these diseases.

As the resident macrophage cells, they act as the first and main form of active immune defense in the central nervous system (CNS). Microglia play a key role after CNS injury, and can have both protective and deleterious effects based on the timing and type of insult (Kreutzberg, G. W. Trends in Neurosciences, 19, 312 (1996); Watanabe, H., et al., Neuroscience Letters, 289, 53 (2000); Polazzi, E., et al., Glia, 36, 271 (2001); Mallard, C., et al., Pediatric Research, 75, 234 (2014); Faustino, J. V., et al., The Journal of Neuroscience: The Official Journal Of The Society For Neuroscience, 31, 12992 (2011); Tabas, I., et al., Science, 339, 166 (2013); and Aguzzi, A., et al., Science, 339, 156 (2013)). Changes in microglial function also affect normal neuronal development and synaptic pruning (Lawson, L. J., et al., Neuroscience, 39, 151 (1990); Giulian, D., et al., The Journal Of Neuroscience: The Official Journal Of The Society For Neuroscience, 13, 29 (1993); Cunningham, T. J., et al., The Journal of Neuroscience: The Official Journal Of The Society For Neuroscience, 18, 7047 (1998); Zietlow, R., et al., The European Journal Of Neuroscience, 11, 1657 (1999); and Paolicelli, R. C., et al., Science, 333, 1456 (2011)). Microglia undergo a pronounced change in morphology from ramified to an amoeboid structure and proliferate after injury. The resulting neuroinflammation disrupts the blood-brain-barrier at the injured site, and cause acute and chronic neuronal and oligodendrocyte death. Hence, targeting pro-inflammatory microglia should be a potent and effective therapeutic strategy. The impaired BBB in neuroinflammatory diseases can be exploited for transport of drug carrying nanoparticles into the brain.

In some embodiments, the dendrimer and DON analog complex compositions are administered in an amount effective to treat cells/tissue with pathogenic dysregulated glutamine-dependent pathways and/or glutamate transmission without any associated toxicity. In preferred embodiments, the dendrimer complex compositions are administered in an amount effective to treat microglial-mediated pathology in the subject in need thereof without any associated toxicity. In further preferred embodiments, the dendrimer complex compositions are administered in an amount effective to treat pathogenic dysregulated glutamine-dependent pathways and/or glutamate transmission in activated microglial cells without any associated toxicity.

In some embodiments, the subject to be treated is a human. In some embodiments, the subject to be treated is a child, or an infant. All the methods can include the step of identifying and selecting a subject in need of treatment, or a subject who would benefit from administration with the compositions.

### 1. Cancer

In some embodiments, the dendrimer and DON analog compositions and formulations thereof are used in a method for treating a cancer in a subject in need of. The method for treating a cancer in a subject in need of including administering to the subject a therapeutically effective amount of the dendrimer and DON analog to treat the cancer, for example, to reduce or prevent one or more symptoms of the cancer.

A cancer in a patient refers to the presence of cells possessing characteristics typical of cancer-causing cells, for example, uncontrolled proliferation, loss of specialized functions, immortality, significant metastatic potential, significant increase in anti-apoptotic activity, rapid growth and proliferation rate, and certain characteristic morphology and cellular markers. In some circumstances, cancer cells will be in the form of a tumor; such cells may exist locally within an animal, or circulate in the blood stream as independent cells, for example, leukemic cells. A tumor refers to all neoplastic cell growth and proliferation, whether malignant or benign, and all precancerous and cancerous cells and tissues. A solid tumor is an abnormal mass of tissue that generally does not contain cysts or liquid areas. A solid tumor may be in the brain, colon, breasts, prostate, liver, kidneys, lungs, esophagus, head and neck, ovaries, cervix, stomach, colon, rectum, bladder, uterus, testes, and pancreas, as non-limiting examples. In some embodiments, the solid tumor regresses or its growth is slowed or arrested after the solid tumor is treated with the presently disclosed methods. In other embodiments, the solid tumor is malignant. In some embodiments, the cancer includes Stage 0 cancer. In some embodiments, the cancer includes Stage I cancer. In some embodiments, the cancer includes Stage II cancer. In some embodiments, the cancer includes Stage III cancer. In some embodiments, the cancer includes Stage IV cancer. In some embodiments, the cancer is refractory and/or metastatic. For example, the cancer may be refractory to treatment with radiotherapy, chemotherapy or monotreatment with immunotherapy. Cancer includes newly diagnosed or recurrent cancers, including without limitation, acute lymphoblastic leukemia, acute myelogenous leukemia, advanced soft tissue sarcoma, brain cancer, metastatic or aggressive breast cancer, breast carcinoma, bronchogenic carcinoma, choriocarcinoma, chronic myelocytic leukemia, colon carcinoma, colorectal carcinoma, Ewing's sarcoma, gastrointestinal tract carcinoma, glioma, glioblastoma multiforme, head and neck squamous cell carcinoma, hepatocellular carcinoma, Hodgkin's disease, intracranial ependymoblastoma, large bowel cancer, leukemia, liver cancer, lung carcinoma, Lewis lung carcinoma, lymphoma, malignant fibrous histiocytoma, a mammary tumor, melanoma, mesothelioma, neuroblastoma, osteosarcoma, ovarian cancer, pancreatic cancer, a pontine tumor, premenopausal breast cancer, prostate cancer, rhabdomyosarcoma, reticulum cell sarcoma, sarcoma, small cell lung cancer, a solid tumor, stomach cancer, testicular cancer, and uterine carcinoma. In some embodiments, the cancer is acute leukemia. In some embodiments, the cancer is acute lymphoblastic leukemia. In some embodiments, the cancer is acute myelogenous leukemia. In some embodiments, the cancer is advanced soft tissue sarcoma. In some embodiments, the cancer is a brain cancer. In some embodiments, the cancer is breast cancer (e.g., metastatic or aggressive breast cancer). In some embodiments, the cancer is breast carcinoma. In some embodiments, the cancer is bronchogenic carcinoma. In some embodiments, the cancer is choriocarcinoma. In some embodiments, the cancer is chronic myelocytic leukemia. In some embodiments, the cancer is a colon carcinoma (e.g., adenocarcinoma). In some embodiments, the cancer is colorectal cancer (e.g., colorectal carcinoma). In some embodiments, the cancer is Ewing's sarcoma. In some embodiments, the cancer is gastrointestinal tract carcinoma. In some embodiments, the cancer is a glioma. In some embodiments, the cancer is glioblastoma multiforme. In some embodiments, the cancer is head and neck squamous cell carcinoma. In some embodiments, the cancer is hepatocellular carcinoma. In some embodiments, the cancer is Hodgkin's disease. In some embodiments, the cancer is intracranial ependymoblastoma. In some embodiments, the cancer is large bowel cancer. In some embodiments, the cancer is leukemia. In some embodiments, the cancer is liver cancer. In some embodiments, the cancer is lung cancer (e.g., lung carcinoma). In some embodiments, the cancer is Lewis lung carcinoma. In some embodiments, the cancer is lymphoma. In some embodiments, the cancer is malignant fibrous histiocytoma. In some embodiments, the cancer includes a mammary tumor. In some embodiments, the cancer is melanoma. In some embodiments, the cancer is mesothelioma. In some embodiments, the cancer is neuroblastoma. In some embodiments, the cancer is osteosarcoma. In some embodiments, the cancer is ovarian cancer. In some embodiments, the cancer is pancreatic cancer. In some embodiments, the cancer includes a pontine tumor. In some embodiments, the cancer is premenopausal breast cancer. In some embodiments, the cancer is prostate cancer. In some embodiments, the cancer is rhabdomyosarcoma. In some embodiments, the cancer is reticulum cell sarcoma. In some embodiments, the cancer is sarcoma. In some embodiments, the cancer is small cell lung cancer. In some embodiments, the cancer includes a solid tumor. In some embodiments, the cancer is stomach cancer. In some embodiments, the cancer is testicular cancer. In some embodiments, the cancer is uterine carcinoma.

Cancers that can be prevented, treated or otherwise diminished by the compositions include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, papillary adenocarcinomas, and gastric cancer (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

The methods and compositions as described are useful for both prophylactic and therapeutic treatment.

Therapeutic treatment involves administering to a subject a therapeutically effective amount of the compositions or pharmaceutically acceptable salts thereof as described after cancer is diagnosed.

In further embodiments, the compositions are used for prophylactic use i.e. prevention, delay in onset, diminution, eradication, or delay in exacerbation of signs or symptoms after onset, and prevention of relapse. For prophylactic use, a therapeutically effective amount of the compounds and compositions or pharmaceutically acceptable salts thereof as described are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms. Prophylactic administration can be used, for example, in the chemopreventative treatment of subjects presenting precancerous lesions, those diagnosed with early-stage malignancies, and for subgroups with susceptibilities (e.g., family, racial, and/or occupational) to particular cancers.

In some embodiments, the subject to be treated is one with one or more solid tumors. A solid tumor is an abnormal mass of tissue that usually does not contain cysts or liquid areas. Solid tumors may be benign (not cancer), or malignant (cancer). Examples of solid tumors are sarcomas, carcinomas, and lymphomas. In preferred embodiments, the compositions and methods are effective in treating one or more symptoms of cancers of the skin, lung, liver, pancreas, brain, kidney, breast, prostate, colon and rectum, bladder, etc. In further embodiment, the tumor is a focal lymphoma or a follicular lymphoma.

### 2. Neurological and Neurodegenerative Diseases

The dendrimer-DON analog compositions and formulations thereof can be used to diagnose and/or to treat one or more neurological and neurodegenerative diseases. The compositions and methods are particularly suited for treating one or more neurological, or neurodegenerative diseases associated with defective metabolism and functions of sphingolipids including sphingomyelin. In some embodiments, the disease or disorder is selected from, but not limited to, some psychiatric disorders (e.g., depression, schizophrenia (SZ), alcohol use disorder, and morphine antinociceptive tolerance) and neurological disorders (e.g., Alzheimer's disease (AD), Parkinson disease (PD)). In one embodiment, the dendrimer complexes are used to treat Alzheimer's Disease (AD) or dementia.

Neurodegenerative diseases are chronic progressive disorders of the nervous system that affect neurological and behavioral function and involve biochemical changes leading to distinct histopathologic and clinical syndromes (Hardy H, et al., Science. 1998; 282:1075-9). Abnormal proteins resistant to cellular degradation mechanisms accumulate within the cells. The pattern of neuronal loss is selective in the sense that one group gets affected, whereas others remain intact. Often, there is no clear inciting event for the disease. The diseases classically described as neurodegenerative are Alzheimer's disease, Huntington's disease, and Parkinson's disease.

Neuroinflammation, mediated by activated microglia and astrocytes, is a major hallmark of various neurological disorders making it a potential therapeutic target (Hagberg, H et al., Annals of Neurology 2012, 71, 444; Vargas, D L et al., Annals of Neurology 2005, 57, 67; and Pardo, C A et al., International Review of Psychiatry 2005, 17, 485). Multiple scientific reports suggest that mitigating neuroinflammation in early phase by targeting these cells can delay the onset of disease and can in turn provide a longer therapeutic window for the treatment (Dommergues, M A et al., Neuroscience 2003, 121, 619; Perry, V H et al., Nat Rev Neurol 2010, 6, 193; Kannan, S et al., Sci. Transl. Med. 2012, 4, 130ra46; and Block, M L et al., Nat Rev Neurosci 2007, 8, 57). The delivery of therapeutics across blood brain barrier is a challenging task. The neuroinflammation causes disruption of blood brain barrier (BBB). The impaired BBB in neuroinflammatory disorders can be utilized to transport drug loaded nanoparticles across the brain (Stolp, H B et al., Cardiovascular Psychiatry and Neurology 2011, 2011, 10; and Ahishali, B et al., International Journal of Neuroscience 2005, 115, 151).

The compositions and methods can also be used to deliver active agents for the treatment of a neurological or neurodegenerative disease or disorder or central nervous system disorder. In preferred embodiments, the compositions and methods are effective in treating, and/or alleviating neuroinflammation associated with a neurological or neurodegenerative disease or disorder or central nervous system disorder. The methods typically include administering to the subject an effective amount of the composition to increase cognition or reduce a decline in cognition, increase a cognitive function or reduce a decline in a cognitive function, increase memory or reduce a decline in memory, increase the ability or capacity to learn or reduce a decline in the ability or capacity to learn, or a combination thereof.

Neurodegeneration refers to the progressive loss of structure or function of neurons, including death of neurons. For example, the compositions and methods can be used to treat subjects with a disease or disorder, such as Parkinson's Disease (PD) and PD-related disorders, Huntington's Disease (HD), Amyotrophic Lateral Sclerosis (ALS), Alzheimer's Disease (AD) and other dementias, Prion Diseases such as Creutzfeldt-Jakob Disease, Corticobasal Degeneration, Frontotemporal Dementia, HIV-Related Cognitive Impairment, Mild Cognitive Impairment, Motor Neuron Diseases (MND), Spinocerebellar Ataxia (SCA), Spinal Muscular Atrophy (SMA), Friedreich's Ataxia, Lewy Body Disease, Alpers' Disease, Batten Disease, Cerebro-Oculo-Facio-Skeletal Syndrome, Corticobasal Degeneration, Gerstmann-Straussler-Scheinker Disease, Kuru, Leigh's Disease, Monomelic Amyotrophy, Multiple System Atrophy, Multiple System Atrophy With Orthostatic Hypotension (Shy-Drager Syndrome), Multiple Sclerosis (MS), Neurodegeneration with Brain Iron Accumulation, Opsoclonus Myoclonus, Posterior Cortical Atrophy, Primary Progressive Aphasia, Progressive Supranuclear Palsy, Vascular Dementia, Progressive Multifocal Leukoencephalopathy, Dementia with Lewy Bodies (DLB), Lacunar syndromes, Hydrocephalus, Wernicke-Korsakoff's syndrome, post-encephalitic dementia, cancer and chemotherapy-associated cognitive impairment and dementia, and depression-induced dementia and pseudodementia. In one embodiment, the neurological disease or disorder is a stress-induced mood disorder, particularly depression.

In further embodiments, the disease or disorder is selected from, but not limited to, injection-localized amyloidosis, cerebral amyloid angiopathy, myopathy, neuropathy, brain trauma, frontotemporal dementia, Pick's disease, multiple sclerosis, prion disorders, diabetes mellitus type 2, fatal familial insomnia, cardiac arrhythmias, isolated atrial amyloidosis, atherosclerosis, rheumatoid arthritis, familial amyloid polyneuropathy, hereditary non-neuropathic systemic amyloidosis, Finnish amyloidosis, lattice corneal dystrophy, systemic AL amyloidosis, and Down syndrome. In preferred embodiments, the disease or disorder is Alzheimer's disease or dementia.

Criteria for assessing improvement in a particular neurological factor include methods of evaluating cognitive skills, motor skills, memory capacity or the like, as well as methods for assessing physical changes in selected areas of the central nervous system, such as magnetic resonance imaging (MRI) and computed tomography scans (CT) or other imaging methods. Such methods of evaluation are well known in the fields of medicine, neurology, psychology and the like, and can be appropriately selected to diagnosis the status of a particular neurological impairment. To assess a change in Alzheimer's disease, or related neurological changes, the selected assessment or evaluation test, or tests, are given prior to the start of administration of the dendrimer compositions. Following this initial assessment, treatment methods for the administration of the dendrimer compositions are initiated and continued for various time intervals. At a selected time interval subsequent to the initial assessment of the neurological defect impairment, the same assessment or evaluation test (s) is again used to reassess changes or improvements in selected neurological criteria.

### 3. Immune Disorders

In some embodiments, compositions of dendrimers conjugated or complexed with one or more DON analogs are administered to a subject with an immune disorder. Exemplary immune disorders include autoimmune or inflammatory diseases or disorders. Autoimmune disease happens when the body's natural defense system cannot effectively differentiate between the body's own cells and foreign cells, causing the body to mistakenly attack normal cells. There are more than 80 types of autoimmune diseases that affect a wide range of body parts. Common autoimmune diseases include rheumatoid arthritis, psoriasis, psoriatic arthritis, systemic lupus erythematosus (SLE), type 1 diabetes, inflammatory bowel disease, and thyroid diseases.

In some embodiments, the compositions can also be used for treatment of autoimmune or inflammatory disease or disorder such as rheumatoid arthritis, systemic lupus erythematosus, alopecia areata, ankylosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Bechet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis—juvenile, discoid lupus, essential mixed cryoglobulinemia, fibromyalgia—fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

In some embodiments, the dendrimer compositions are administered in an amount and dosing regimen effective to induce a desirable immunological outcome in a subject in need thereof. Administration of the compositions leads to an improvement, or re-balancing of immune environment in an individual with an immunological disorder.

### 4. Viral Infections

In some embodiments, the dendrimer compositions and formulations thereof are used in a method for treating one or more viral infections. In some embodiments, the dendrimer compositions and formulations thereof are used for reducing or inhibiting viral replication, viral load, and/or viral release, particularly in cases where activated microglia and astrocytes are targeted/infected by the virus.

In some embodiments, the dendrimer-DON analog compositions are effective in reducing an inflammatory response again a viral infection where virus-specific immune response is implicated as a mediator of neuronal damage. For example, the compositions are effective in reducing lymphocyte proliferation in the draining lymph nodes, decreased leukocyte infiltration into the CNS, lower levels of inflammatory cytokines this reducing one of more symptoms associated with elevated inflammatory response such as a cytokine storm.

### C. Dosage and Effective Amounts

Dosage and dosing regimens are dependent on the severity and location of the disorder or injury and/or methods of administration, and is known to those skilled in the art. A therapeutically effective amount of the dendrimer composition used in the treatment of a neurological disease is typically sufficient to reduce or alleviate one or more symptoms of the neurological disease. Preferably, the agents do not target or otherwise modulate the activity or quantity of healthy cells not within or associated with the diseased or target tissues, or do so at a reduced level compared to target cells including activated microglial cells in the CNS. In this way, by-products and other side effects associated with the compositions are reduced. Administration of the compositions leads to an improvement, or enhancement, of neurological function in an individual with a neurological disease, neurological injury, or age-related neuronal decline or impairment. In some in vivo approaches, the dendrimer complexes are administered to a subject in a therapeutically effective amount to stimulate or induce neural mitosis leading to the generation of new neurons, providing a neurogenic effect. Also provided are effective amounts of the compositions to prevent, reduce, or terminate deterioration, impairment, or death of an individual's neurons, neurites and neural networks, providing a neuroprotective effect.

A therapeutically effective amount of the dendrimer composition used in the treatment of a cancer is typically sufficient to reduce or alleviate one or more symptoms of the cancer. Symptoms of cancer may be physical, such as tumor burden, or biological such as proliferation of cancer cells. Accordingly, the amount of dendrimer complex can be effective to, for example, kill tumor cells or inhibit proliferation or metastasis of the tumor cells. Preferably the dendrimer composition including one or more active agents, for example immunomodulatory agents, are preferentially delivered to cells in and around tumor tissues, for example, cancerous cells or immune cells associated with tumor tissues (e.g. M2 macrophages). Preferably the active agents do not target or otherwise modulate the activity or quantity of healthy cells not within or associated with tumor tissues, or do so at a reduced level compared to cancer or cancer-associated cells. In this way, by-products and other side effects associated with the compositions are reduced, preferably leading directly or indirectly to cancer cell death. In some embodiments, the active agent directly or indirectly reduces cancer cell migration, angiogenesis, immune escape, radioresistance, or a combination thereof. In some embodiments, the active agent directly or indirectly induces a change in the cancer cell itself or its microenvironment that reduces suppression or induces activation of an immune response against the cancer cells. In some in vivo approaches, the dendrimer complexes are administered to a subject in a therapeutically effective amount to reduce tumor size. In some embodiments, an effective amount of the composition is used to put cancer in remission and/or keep the cancer in remission. Also provided are effective amounts of the compositions to reduce or stop cancer stem cell proliferation.

The actual effective amounts of dendrimer complex can vary according to factors including the specific agent administered, the particular composition formulated, the mode of administration, and the age, weight, condition of the subject being treated, as well as the route of administration and the disease or disorder. The dose of the compositions can be from about 0.01 to about 100 mg/kg body weight, from about 0.1 mg/kg to about 10 mg/kg, and from about 0.5 mg to about 5 mg/kg body weight. Generally, for intravenous injection or infusion, the dosage may be lower than for oral administration.

In general, the timing and frequency of administration will be adjusted to balance the efficacy of a given treatment or diagnostic schedule with the side-effects of the given delivery system. Exemplary dosing frequencies include continuous infusion, single and multiple administrations such as hourly, daily, weekly, monthly or yearly dosing.

The compositions can be administered daily, biweekly, weekly, every two weeks or less frequently in an amount to provide a therapeutically effective increase in the blood level of the therapeutic agent. Where the administration is by other than an oral route, the compositions may be delivered over a period of more than one hour, e.g., 3-10 hours, to produce a therapeutically effective dose within a 24-hour period. Alternatively, the compositions can be formulated for controlled release, wherein the composition is administered as a single dose that is repeated on a regimen of once a week, or less frequently.

Dosage can vary, and can be administered in one or more dose administrations daily, for one or several days. Guidance can be found in the literature for appropriate dosages for given classes of pharmaceutical products. Optimal dosing schedules can be calculated from measurements of drug accumulation in the body of the subject or patient. Persons of ordinary skill can easily determine optimum dosages, dosing methodologies and repetition rates. Optimum dosages can vary depending on the relative potency of individual pharmaceutical compositions, and can generally be estimated based on EC₅₀s found to be effective in in vitro and in vivo animal models.

In some embodiments, the regimen includes one or more cycles of a round of therapy followed by a drug holiday (e.g., no drug). The drug holiday can be 1, 2, 3, 4, 5, 6, or 7 days; or 1, 2, 3, 4 weeks, or 1, 2, 3, 4, 5, or 6 months.

### D. Combination Therapies and Procedures

The dendrimer and DON analog compositions can be administered alone or in combination with one or more conventional therapies. Examples of preferred additional therapeutic agents include other conventional therapies known in the art for treating the desired disease, disorder or condition.

In the context of Alzheimer's disease, the other therapeutic agents can include one or more of acetylcholinesterase inhibitors (such as tacrine, rivastigmine, galantamine or donepezil), beta-secretase inhibitors such as JNJ-54861911, antibodies such as aducanumab, agonists for the 5-HT2A receptor such as pimavanserin, sargramostim, AADvac1, CAD106, CNP520, gantenerumab, solanezumab, and memantine.

In the context of Dementia with Lewy Bodies, the other therapeutic agents can include one or more of acetylcholinesterase inhibitors such as tacrine, rivastigmine, galantamine or donepezil; the N-methyl d-aspartate receptor antagonist memantine; dopaminergic therapy, for example, levodopa or selegiline; antipsychotics such as olanzapine or clozapine; REM disorder therapies such as clonazepam, melatonin, or quetiapine; anti-depression and antianxiety therapies such as selective serotonin reuptake inhibitors (citalopram, escitalopram, sertraline, paroxetine, etc.) or serotonin and noradrenaline reuptake inhibitors (venlafaxine, mirtazapine, and bupropion) (see, e.g., Macijauskiene, et al., Medicina (Kaunas), 48(1):1-8 (2012)).

Exemplary neuroprotective agents are also known in the art in include, for example, glutamate antagonists, antioxidants, and NMDA receptor stimulants. Other neuroprotective agents and treatments include caspase inhibitors, trophic factors, anti-protein aggregation agents, therapeutic hypothermia, and erythropoietin.

Other common active agents for treating neurological dysfunction include amantadine and anticholinergics for treating motor symptoms, clozapine for treating psychosis, cholinesterase inhibitors for treating dementia, and modafinil for treating daytime sleepiness.

### 1. Cancer Treatment

In some embodiments, compositions of dendrimers conjugated or complexed with one or more 6-diazo-5-oxo-L-norleucine (DON) analogs are administered in combination with one or more conventional therapies, for example, a conventional cancer therapy. In some embodiments, the conventional therapy includes administration of one or more of the compositions in combination with one or more additional active agents. The combination therapies can include administration of the active agents together in the same admixture, or in separate admixtures. Therefore, in some embodiments, the pharmaceutical composition includes two, three, or more active agents. Such formulations typically include an effective amount of an immunomodulatory agent targeting tumor microenvironment. The additional active agent(s) can have the same, or different mechanisms of action. In some embodiments, the combination results in an additive effect on the treatment of the cancer. In some embodiments, the combinations result in a more than additive effect on the treatment of the disease or disorder.

In some embodiments, the formulation is formulated for intravenous, subcutaneous, or intramuscular administration to the subject, or for enteral administration. In some embodiments, the formulation is administered prior to, in conjunction with, subsequent to, or in alternation with treatment with one or more additional therapies or procedures. In some embodiments the additional therapy is performed between drug cycles or during a drug holiday that is part of the composition dosage regime. For example, in some embodiments, the additional therapy or procedure is surgery, a radiation therapy, or chemotherapy.

Additional therapeutic agents include conventional cancer therapeutics such as chemotherapeutic agents, cytokines, chemokines, and radiation therapy. The majority of chemotherapeutic drugs can be divided into alkylating agents, antimetabolites, anthracyclines, plant alkaloids, topoisomerase inhibitors, and other antitumour agents. These drugs affect cell division or DNA synthesis and function in some way. Additional therapeutics include monoclonal antibodies and the tyrosine kinase inhibitors e.g., imatinib mesylate (GLEEVEC® or GLIVEC®), which directly targets a molecular abnormality in certain types of cancer (chronic myelogenous leukemia, gastrointestinal stromal tumors).

Representative chemotherapeutic agents include, but are not limited to, amsacrine, bleomycin, busulfan, camptothecin, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epipodophyllotoxins, epirubicin, etoposide, etoposide phosphate, fludarabine, fluorouracil, gemcitabine, hydroxycarb amide, idarubicin, ifosfamide, innotecan, leucovorin, liposomal doxorubicin, liposomal daunorubici, lomustine, mechlorethamine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, teniposide, tegafur-uracil, temozolomide, teniposide, thiotepa, tioguanine, topotecan, treosulfan, vinblastine, vincristine, vindesine, vinorelbine, vorinostat, taxol, trichostatin A and derivatives thereof, trastuzumab (HERCEPTIN®), cetuximab, and rituximab (RITUXAN® or MABTHERA®), bevacizumab (AVASTIN®), and combinations thereof. Representative pro-apoptotic agents include, but are not limited to, fludarabinetaurosporine, cycloheximide, actinomycin D, lactosylceramide, 15d-PGJ(2)5 and combinations thereof.

In some embodiments, the compositions and methods are used prior to or in conjunction with an immunotherapy such inhibition of checkpoint proteins such as components of the PD-1/PD-L1 axis or CD28-CTLA-4 axis using one or more immune checkpoint modulators (e.g., PD-1 antagonists, PD-1 ligand antagonists, and CTLA4 antagonists), adoptive T cell therapy, and/or a cancer vaccine. Exemplary immune checkpoint modulators used in immunotherapy include Pembrolizumab (anti-PD1 mAb), Durvalumab (anti-PDL1 mAb), PDR001 (anti-PD1 mAb), Atezolizumab (anti-PDL1 mAb), Nivolumab (anti-PD1 mAb), Tremelimumab (anti-CTLA4 mAb), Avelumab (anti-PDL1 mAb), and RG7876 (CD40 agonist mAb).

Methods of adoptive T cell therapy are known in the art and used in clinical practice. Generally adoptive T cell therapy involves the isolation and ex vivo expansion of tumor specific T cells to achieve greater number of T cells than what could be obtained by vaccination alone. The tumor specific T cells are then infused into patients with cancer in an attempt to give their immune system the ability to overwhelm remaining tumor via T cells, which can attack and kill the cancer. Several forms of adoptive T cell therapy can be used for cancer treatment including, but not limited to, culturing tumor infiltrating lymphocytes or TIL; isolating and expanding one particular T cell or clone; and using T cells that have been engineered to recognize and attack tumors. In some embodiments, the T cells are taken directly from the patient's blood. Methods of priming and activating T cells in vitro for adaptive T cell cancer therapy are known in the art. See, for example, Wang, et al, Blood, 109(11):4865-4872 (2007) and Hervas-Stubbs, et al, J. Immunol., 189(7):3299-310 (2012).

Historically, adoptive T cell therapy strategies have largely focused on the infusion of tumor antigen specific cytotoxic T cells (CTL) which can directly kill tumor cells. However, CD4+ T helper (Th) cells such as Th1, Th2, Tfh, Treg, and Th17 can also be used. Th can activate antigen-specific effector cells and recruit cells of the innate immune system such as macrophages and dendritic cells to assist in antigen presentation (APC), and antigen primed Th cells can directly activate tumor antigen-specific CTL. As a result of activating APC, antigen specific Th₁ have been implicated as the initiators of epitope or determinant spreading which is a broadening of immunity to other antigens in the tumor. The ability to elicit epitope spreading broadens the immune response to many potential antigens in the tumor and can lead to more efficient tumor cell kill due to the ability to mount a heterogeneic response. In this way, adoptive T cell therapy can used to stimulate endogenous immunity.

In some embodiments, the T cells express a chimeric antigen receptor (CARs, CAR T cells, or CARTs). Artificial T cell receptors are engineered receptors, which graft a particular specificity onto an immune effector cell. Typically, these receptors are used to graft the specificity of a monoclonal antibody onto a T cell and can be engineered to target virtually any tumor associated antigen. First generation CARs typically had the intracellular domain from the CD3 ζ-chain, which is the primary transmitter of signals from endogenous TCRs. Second generation CARs add intracellular signaling domains from various costimulatory protein receptors (e.g., CD28, 41BB, ICOS) to the cytoplasmic tail of the CAR to provide additional signals to the T cell, and third generation CARs combine multiple signaling domains, such as CD3z-CD28-41BB or CD3z-CD28-OX40, to further enhance effectiveness.

In some embodiments, the compositions and methods are used prior to or in conjunction with a cancer vaccine, for example, a dendritic cell cancer vaccine. Vaccination typically includes administering a subject an antigen (e.g., a cancer antigen) together with an adjuvant to elicit therapeutic T cells in vivo. In some embodiments, the cancer vaccine is a dendritic cell cancer vaccine in which the antigen delivered by dendritic cells primed ex vivo to present the cancer antigen. Examples include PROVENGE® (sipuleucel-T), which is a dendritic cell-based vaccine for the treatment of prostate cancer (Ledford, et al., Nature, 519, 17-18 (5 Mar. 2015). Such vaccines and other compositions and methods for immunotherapy are reviewed in Palucka, et al., Nature Reviews Cancer, 12, 265-277 (April 2012).

In some embodiments, the compositions and methods are used prior to or in conjunction with surgical removal of tumors, for example, in preventing primary tumor metastasis. In some embodiments, the compositions and methods are used to enhance body's own anti-tumor immune functions.

### E. Controls

The therapeutic result of the dendrimer complex compositions including one or more 6-diazo-5-oxo-L-norleucine (DON) analogs can be compared to a control. Suitable controls are known in the art and include, for example, an untreated subject or untreated cells. A typical control is a comparison of a condition or symptom of a subject prior to and after administration of the targeted agent. The condition or symptom can be a biochemical, molecular, physiological, or pathological readout. For example, the effect of the composition on a particular symptom, pharmacologic, or physiologic indicator can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects). In some embodiments, the effect of the treatment is compared to a conventional treatment that is known the art.

### VI. Kits

The compositions can be packaged in kit. The kit can include a single dose or a plurality of doses of a composition including one or more 6-diazo-5-oxo-L-norleucine (DON) analogs encapsulated in, associated with, or conjugated to a dendrimer, and instructions for administering the compositions. Specifically, the instructions direct that an effective amount of the composition be administered to an individual with a particular disease, disorder or impairment as indicated. The composition can be formulated as described above with reference to a particular treatment method and can be packaged in any convenient manner.

The present invention will be further understood by reference to the following non-limiting examples.

### EXAMPLES

### Example 1: Synthetic Methods for Glutamine Antagonists

### Synthetic Methods for Glutamine Antagonists

FIG. 1 is a scheme showing the synthetic scheme for compound I. (i) Fmoc-Cl, NaHCO₃, dioxane, 0° C., 2 h, 67%; (ii) isopropyl (S)-2-amino-6-diazo-5-oxohexanoate, phenyl dichlorophosphate, 2,4,6-trimethylpyridine, THF, 0° C. to RT, 5 h, 41%; (iii) piperidine, DCM, RT, 2.5 h, 73%.

FIG. 2 is a scheme showing the synthetic scheme for compound II. (i) HATU, DIEA, Fmoc-L-Leu-OH, DCM, 0° C. to RT, 20 h, 76%; (ii) piperidine, DCM, RT, 2 h, 93%; (iii) 5-(Fmoc-amino)-3-oxapentanoic acid, HATU, DIEA, DMF, 0° C. to RT, 1 h, 70%; (iv) piperidine, DCM, RT, 2 h, 93%.

FIG. 3 is a scheme showing the synthetic scheme for compound III. (i) Fmoc-9-amino-4,7-dioxanonanoic acid, HATU, DIEA, DMF, 0° C. to RT, 1 h, 67%; (ii) piperidine, DCM, RT, 3 h, 89%.

FIG. 4 is a scheme showing the synthetic scheme for compound IV. (i) HATU, DIEA, Fmoc-L-Leu-OH, DCM, 0° C. to RT, 20 h, 96%; (ii) diethylamine, DCM, RT, 1.5 h, 88%; (iii) Fmoc-9-amino-4,7-dioxanonanoic acid, HATU, DIEA, DMF, 0° C. to RT, 1 h, 86%; (iv) piperidine, DCM, RT, 1 h, 87%.

FIG. 5 is a scheme showing the synthetic scheme for compound V. (i) 5-(Fmoc-amino)-3-oxapentanoic acid, HATU, DIEA, DMF, 0° C. to RT, 1 h, 89%; (ii) piperidine, DCM, RT, 1 h, 74%.

FIG. 6 is a scheme showing the synthetic scheme for compound VI. (i) Allyl bromide, Cs2CO3, MeCN, RT, 1 h, 96%; (ii) TFA, DCM; (iii) Boc-L-Phe-OH, HATU, DIEA, DMF, RT, 3 h, 88%; (iv) TFA, DCM; (v) a DIEA, DCM; b (S)-2-(Fmoc-amino)-6-diazo-5-oxohexanoic acid, EDC, HOBt, DCM, RT, 2 h, 71%; (vi) diethylamine, DCM, 75%; (vii) Fmoc-L-Leu-OH, DIEA, HBTU, DCM, RT, 2 h, 47%; (viii) Pd(PPh₃)₄, dimedone, THF, 63%.

FIG. 7 is a scheme showing the synthetic scheme for compound VII. (i) Allyl bromide, Cs2CO3, MeCN, 50° C., 1 h, 96%; (ii) TFA, DCM; (iii) Boc-L-Phe-OH, HATU, DIEA, DMF, RT, 3 h, 95%; (iv) TFA, DCM; (v) a DIEA, DCM; b (S)-2-(Fmoc-amino)-6-diazo-5-oxohexanoic acid, EDC, HOBt, DCM, RT, 2 h, 60%; (vi) diethylamine, MeCN; (vii) Fmoc-L-Leu-OSu, MeCN, RT, 3 h, 48%; (viii) Pd(PPh₃)₄, dimedone, THF, RT, 1 h, 74%.

FIG. 8 is a scheme showing the synthetic scheme for compound VIII. (i) HATU, DIEA, H-Gly-OAll*HCl, DCM, 0° C. to RT, 2.5 h, 77%; (ii) diethylamine, DCM, RT, 2.5 h; (iii) (((4-nitrophenoxy)carbonyl)oxy)(phenyl)methyl pivalate, DMF, RT, 4 h, 54% over 2 steps; (iv) Pd(PPh3)4, phenyl silane, DCM, RT, 0.5 h; (v) HATU, DIEA, H₂N-PEG5-COOAll*TFA salt, DMF, 0° C. to RT, 4 h; (vi) Pd(PPh₃)₄, phenyl silane, DCM, RT, 0.5 h.

FIG. 9 is a scheme showing the synthetic scheme for compounds IX and X. (i) LiHMDS, Fmoc-Cl, THF, −78° C. to rt; (ii) TMS-diazomethane, n-BuLi, THF, −78° C.; (iii) piperidine, DMF, rt; (iv) dimedone, Pd(PPh₃)₄, THF; (v) HATU, DCM, DIEA, 0° C.; (vi) piperidine, DMF, rt; (vii) C₈H₁₇OH, DCC, DMAP, DCM, rt; (viii) Pd(PPh₃)₄, PhSiH₃, DCM, rt; (ix) HATU, DIEA, DCM, 0° C.; (x) piperidine, DMF, rt; (xi) HO-PEG4-CH₂CO₂H, HOBt, EDC, DIEA, THF, rt.

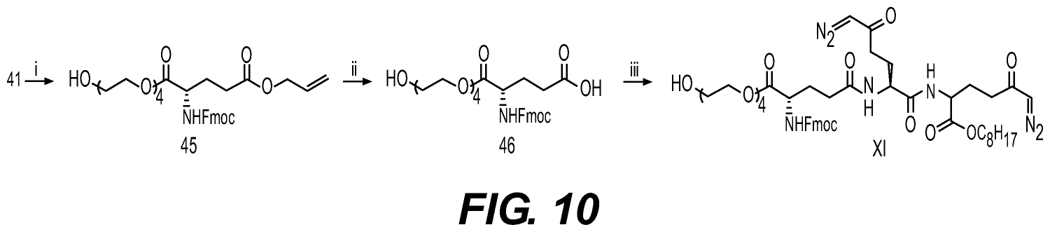

FIG. 10 is a scheme showing the synthetic scheme for compounds XI. (i) 2,2′-((oxybis(ethane-2,1-diyl))bis(oxy))bis(ethan-1-ol), DCC, DMAP, DCM, rt; (ii) Pd(PPh₃)₄, PhSiH₃, DCM, 0° C.; (iii) Compound 40, HATU, DIEA, DCM, 0° C.

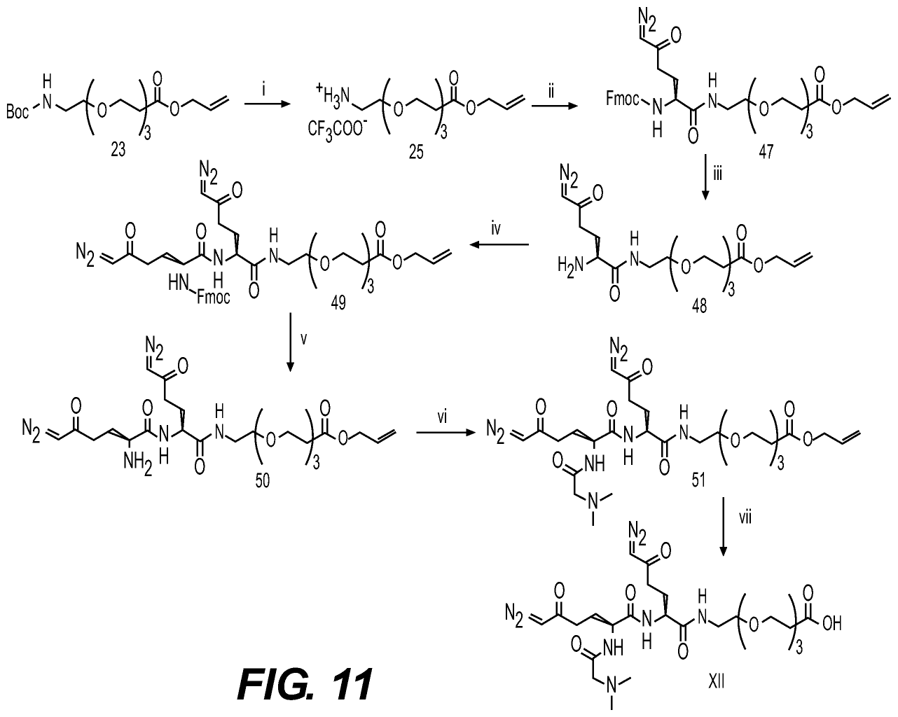

FIG. 11 is a scheme showing the synthetic scheme for compounds XII. (i) TFA, DCM, 0° C. to rt, 1 h; (ii) Fmoc-DON-OH, HATU, DIPEA, DCM, 0° C. to rt, 2 h, 98%; (iii) Et₂NH, DCM, rt, 2 h; (iv) Fmoc-DON-OH, HATU, DIPEA, DCM, 0° C. to rt, 2 h, 42%; (v) Et₂NH, DMF, rt, 2 h; (vi) 2,5-dioxopyrrolidin-1-yl dimethylglycinate, DMF, rt, 2 h, 53%; (vii) Pd(PPh₃)₄, dimedone, THF, rt, 1 h, 31%. Reaction conditions: i) TFA, DCM, 0° C. to rt, 1 h; ii) Fmoc-DON-OH, HATU, DIPEA, DCM, 0° C. to rt, 2 h, 98%; iii) Et₂NH, DCM, rt, 2 h; iv) Fmoc-DON-OH, HATU, DIPEA, DCM, 0° C. to rt, 2 h, 42%; v) Et₂NH, DMF, rt, 2 h; vi) 2,5-dioxopyrrolidin-1-yl dimethylglycinate, DMF, rt, 2 h, 53%; vii) Pd(PPh₃)₄, dimedone, THF, rt, 1 h, 31%.

### (9H-Fluoren-9-yl)methyl (4-hydroxybutyl)carbamate (2)

As shown in FIG. 1, 4-Aminobutanol (967 mg, 1.00 mL, 10.8 mmol, 1.0 equiv.) was dissolved in dioxane (30 mL) and this solution was cooled to 0° C. Sat. NaHCO₃ (15 mL) was added dropwise during 5 minutes followed by Fmoc chloride (3.08 g, 11.9 mmol, 1.1 equiv.) and reaction mixture was stirred at rt for 1 h. The reaction was quenched with sat. NaCl (50 mL). The aqueous phase was extracted with ethyl acetate (3×50 mL), the combined organic layers were washed with dest. water (40 mL), brine (40 mL) and dried over anhydrous MgSO₄. The organic solvent was evaporated in vacuo. The residue was chromatographed on silica gel (EtOAc/hexane 1:1) to afford the desired product 2 (2.05 g, 67%) as a colorless solid.

### Isopropyl (2S)-2-(((4-((((9H-fluoren-9-yl)methoxy)carbonyl)amino)butoxy)(phenoxy) phosphoryl)amino)-6-diazo-5-oxohexanoate (3)

As shown in FIG. 1, Phenyl dichlorophosphate (1.05 mL, 7.05 mmol, 1.5 equiv.) was dissolved in anhydrous THF (8.5 mL) under inert atmosphere. The solution was cooled to 0° C. and 2,4,6-trimethylpyridine (9.28 mL, 70.3 mmol, 15 equiv.) was slowly added followed by solution of 2 (1.46 g, 4.70 mmol, 1.0 equiv.) in anhydrous THF (8.5 mL). The reaction mixture was stirred at 0° C. for 20 min. The solution of isopropyl (S)-2-amino-6-diazo-5-oxohexanoate (1.00 g, 4.70 mmol, 1.0 equiv.) in anhydrous THF (3.5 mL) was added and reaction mixture was stirred at 0° C. for 30 min and at rt for 2 h. The solvent was evaporated in vacuo and the crude product was chromatographed on silica gel (gradient CHCl₃/MeOH 30:1→15:1) to afford the product 3 (1.26 g, 41%) as a yellow amorphous solid.

### Isopropyl (2S)-2-(((4-aminobutoxy)(phenoxy)phosphoryl)amino)-6-diazo-5-oxohexanoate (Structure I)

As shown in FIG. 1, The compound 3 (340 mg, 0.513 mmol, 1.0 equiv.) was dissolved in anhydrous DCM (7 mL) under inert atmosphere. Piperidine (1.00 mL, 10.3 mmol, 20 equiv.) was added and the reaction mixture was stirred at rt for 2 h. The solvent was evaporated and the residue was chromatographed on silica gel (CHCl₃/MeOH 1:1+2% Et₃N) to afford the desired product I (165 mg, 73%) as a yellowish oil. The prepared product I is very unstable and should be stored at −80° C.

### t-Butyl (11S,14S)-14-(4-diazo-3-oxobutyl)-1-(9H-fluoren-9-yl)-11-isobutyl-3,9,12-trioxo-2,7-dioxa-4,10,13-triazapentadecan-15-oate (7)

As shown in FIG. 2, FmocHN-CH₂CH₂—OCH₂COOH (0.24 g, 0.71 mmol, 1.1 equiv.) and HATU (0.27 g, 0.71 mmol, 1.1 equiv.) were dissolved in DMF (5 mL) under inert atmosphere. The reaction mixture was cooled to 0° C. and DIEA (0.37 mL, 2.1 mmol, 3.0 equiv.) was added. Finally, solution of H-Leu-DON-OtBu 6 (0.22 g, 0.65 mmol, 1.0 equiv.) in DMF (1 mL) was added. The resulting mixture was stirred for 15 min. at 0° C. and overnight at room temperature. Then, solvent was evaporated, EtOAc was added (20 mL) and the organic phase was washed with distilled H₂O (15 mL), 10% aq. KHSO₄ (2×15 mL), sat. NaHCO₃ (2×15 mL) and brine (10 mL). The organic phase was then dried over anhydrous MgSO₄ and solvent was evaporated. The residue was purified by column chromatography (DCM/acetone 2:1, R_{f}=0.28). Reaction afforded 0.30 g (70%) of product as yellowish amorphous solid.

### t-Butyl (S)-2-((S)-2-(2-(2-aminoethoxy)acetamido)-4-methylpentanamido)-6-diazo-5-oxohexanoate (Structure II)

As shown in FIG. 2, Compound 7 (0.29 g, 0.44 mmol, 1.0 equiv.) was dissolved in DCM (2 mL) under inert atmosphere. Then, piperidine (0.43 mL, 4.4 mmol, 10 equiv.) was added and the resulting mixture was stirred for 2 hours at room temperature. The solvent was evaporated and the residue was purified by column chromatography (DCM/MeOH/NH₃ 100:20:1, R_{f}=0.16). Reaction afforded 0.18 g (93%) of product as yellowish amorphous solid.

### t-Butyl (15S,18S)-18-(4-diazo-3-oxobutyl)-1-(9H-fluoren-9-yl)-15-isobutyl-3,13,16-trioxo-2,7,10-trioxa-4,14,17-triazanonadecan-19-oate (9)

As shown in FIG. 3, FmocHN—(CH₂CH₂-0)₂CH₂CH₂COOH (0.29 g, 0.73 mmol, 1.1 equiv.) and HATU (0.28 g, 0.73 mmol, 1.1 equiv.) were dissolved in DMF (5 mL) under inert atmosphere. The reaction mixture was cooled to 0° C. and DIEA (0.37 mL, 2.1 mmol, 3.0 equiv.) was added. Finally, solution of H-Leu-DON-OtBu 6 (0.23 g, 0.66 mmol, 1.0 equiv.) in DMF (1 mL) was added. The resulting mixture was stirred for 15 min. at 0° C. and overnight at room temperature. Then, solvent was evaporated, EtOAc was added (20 mL) and the organic phase was washed with distilled H₂O (15 mL), 10% aq. KHSO4 (2×15 mL), sat. NaHCO₃ (2×15 mL) and brine (10 mL). The organic phase was then dried over anhydrous MgSO₄ and solvent was evaporated. The residue was purified by column chromatography (DCM/acetone 2:1, R_{f}=0.19). Reaction afforded 0.32 g (67%) of product as yellowish amorphous solid.

### t-Butyl (2S,5S)-15-amino-2-(4-diazo-3-oxobutyl)-5-isobutyl-4,7-dioxo-10,13-dioxa-3,6-diazapentadecanoate (Structure III)

As shown in FIG. 3, Compound 9 (65 mg, 0.09 mmol, 1.0 equiv.) was dissolved in DCM (1 mL) under inert atmosphere. Then, piperidine (90 μL, 0.9 mmol, 10 equiv.) was added and the resulting mixture was stirred for 3 hours at room temperature. The solvent was evaporated and the residue was purified by column chromatography (DCM/MeOH 5:1, R_{f}=0.25). Reaction afforded 40 mg (89%) of product as yellowish amorphous solid.

### Isopropyl (S)-2-((S)-2-((((9H-fluoren-9-yl)methoxy)carbonyl)amino)-4-methylpentanamido)-6-diazo-5-oxohexanoate (11)

Compound 11 was prepared according to the literature (Rais, R. et al., J. Med. Chem. 2016, 59 (18), 8621-8633.

### Isopropyl (S)-2-((S)-2-amino-4-methylpentanamido)-6-diazo-5-oxohexanoate (12)

Compound 12 was prepared according to the literature (Rais, R. et al., J. Med. Chem. 2016, 59 (18), 8621-8633.

### Isopropyl (15S,18S)-18-(4-diazo-3-oxobutyl)-1-(9H-fluoren-9-yl)-15-isobutyl-3,13,16-trioxo-2,7,10-trioxa-4,14,17-triazanonadecan-19-oate (13)

As shown in FIG. 4, FmocHN—(CH₂CH₂-0)₂CH₂CH₂COOH (0.34 g, 0.84 mmol, 1.1 equiv.) and HATU (0.32 g, 0.84 mmol, 1.1 equiv.) were dissolved in anhydrous DMF (5 mL) under inert atmosphere. The reaction mixture was cooled to 0° C. and DIEA (0.40 mL, 2.3 mmol, 3.0 equiv.) was added. Finally, solution of H-Leu-DON-OiPr 12 (0.25 g, 0.77 mmol, 1.0 equiv.) in DMF (1 mL) was added. The resulting mixture was stirred for 15 min. at 0° C. and 1 hour at room temperature. Then, solvent was evaporated, DCM was added (50 mL) and the organic phase was washed with 10% aq. KHSO4 (25 mL), distilled H₂O (25 mL), sat. NaHCO₃ (25 mL), distilled H₂O (25 mL), and brine (25 mL). The organic phase was then dried over anhydrous MgSO₄ and solvent was evaporated. The residue was purified by column chromatography (DCM/MeOH 40:1, R_{f}=0.18). Reaction afforded 0.47 g (86%) of product as yellowish amorphous solid.

### Isopropyl (2S,5S)-15-amino-2-(4-diazo-3-oxobutyl)-5-isobutyl-4,7-dioxo-10,13-dioxa-3,6-diazapentadecanoate (Structure IV)

As shown in FIG. 4, Compound 13 (0.41 g, 0.58 mmol, 1.0 equiv.) was dissolved in anhydrous DCM (1 mL) under inert atmosphere. Then, piperidine (285 μL, 2.89 mmol, 5 equiv.) was added and the resulting mixture was stirred for 1 hour at room temperature. The solvent was evaporated and the residue was purified by column chromatography (DCM/MeOH 5:1+1% Et₃N, R_{f}=0.44). Reaction afforded 218 mg (78%) of product as yellowish amorphous solid.

### Isopropyl (11S,14S)-14-(4-diazo-3-oxobutyl)-1-(9H-fluoren-9-yl)-11-isobutyl-3,9,12-trioxo-2,7-dioxa-4,10,13-triazapentadecan-15-oate (14)

As shown in FIG. 5, FmocHN-CH₂CH₂—OCH₂COOH (0.29 g, 0.84 mmol, 1.1 equiv.) and HATU (0.32 g, 0.84 mmol, 1.1 equiv.) were dissolved in anhydrous DMF (5 mL) under inert atmosphere. The reaction mixture was cooled to 0° C. and DIEA (0.40 mL, 2.30 mmol, 3.0 equiv.) was added. Finally, solution of H-Leu-DON-OiPr (12) (0.25 g, 0.77 mmol, 1.0 equiv.) in anhydrous DMF (1 mL) was added. The resulting mixture was stirred for 10 min. at 0° C. and 1 hour at room temperature. Then, solvent was evaporated, DCM was added (50 mL) and the organic phase was washed with 10% aq. KHSO₄ (25 mL), distilled H₂O (25 mL), sat. NaHCO₃ (25 mL), distilled H₂O (25 mL) and brine (25 mL). The organic phase was then dried over anhydrous MgSO₄ and solvent was evaporated. The residue was purified by column chromatography (DCM/MeOH 40:1, R_{f}=0.49). Reaction afforded 0.44 g (89%) of product as yellowish solid.

### Isopropyl (S)-2-((S)-2-(2-(2-aminoethoxy)acetamido)-4-methylpentanamido)-6-diazo-5-oxohexanoate (Structure V)

As shown in FIG. 5, Compound 14 (0.39 g, 0.60 mmol, 1.0 equiv.) was dissolved in anhydrous DCM (1 mL) under inert atmosphere. Then, piperidine (0.30 mL, 3.12 mmol, 5 equiv.) was added and the resulting mixture was stirred for 1 hour at room temperature. The solvent was evaporated and the residue was purified by column chromatography (DCM/MeOH 5:1+1% Et₃N, R_{f}=0.42). Reaction afforded 0.19 g (74%) of product as yellowish amorphous solid.

### Allyl 2, 2-dimethyl-4-oxo-3,8,11,14,17-pentaoxa-5-azaicosan-20-oate (16)

As shown in FIG. 6, 2,2-Dimethyl-4-oxo-3,8,11,14,17-pentaoxa-5-azaicosan-20-oic acid 15 (1.10 g, 3.0 mmol) and Cs₂CO₃ (1.47 g, 4.5 mmol) in MeCN (20 mL) was treated with allyl bromide (0.39 mL, 4.5 mmol). The reaction mixture was stirred at RT for 1 h, diluted with EtOAc (20 mL) and precipitated salts were filtered off. The residue was chromatographed on a silica gel column in 5% MeOH/CHCl₃. Yield 1.19 g (98%) of syrup. Analytical data have been described in U.S. Patent Publication No. US2012/225089.

### Allyl (S)-6-benzyl-2,2-dimethyl-4,7-dioxo-3,11,14,17,20-pentaoxa-5,8-diaza-tricosan-23-oate (18)

As shown in FIG. 6, a solution of 16 (1.22 g, 3.01 mmol) in CH₂Cl₂ (6 mL) was treated with TFA (3 mL) at 0° C. Reaction mixture was stirred at RT for 30 min, then evaporated and codistilled with toluene (10 mL). The resulting syrupy trifluroacetate salt of 17 was kept in vacuo. In the meantime, a solution of Boc-L-Phe (878 mg, 3.31 mmol) and HATU (1.37 g, 3.61 mmol) in anhydrous DMF (3 mL) was treated with DIPEA (2.62 mL, 15.04 mmol) at 0° C. and stirred for 10 min. The previously prepared trifluroacetate salt of 17 was dissolved in anhydrous DMF (5 mL) and added to the mixture, which was then stirred at RT for 3 h. The solution was concentrated and the residue was subjected to RP flash chromatography (100 g HP C18 Aq column, 50 mL/min, gradient 0%-100% acetonitrile/water in 25 min, detection 220 nm). Yield 1.49 g (90%) of syrup.

### Allyl (5S,8S)-8-benzyl-5-(4-diazo-3-oxobutyl)-1-(9H-fluoren-9-yl)-3,6,9-tri-oxo-2,13,16,19,22-pentaoxa-4,7,10-triazapentacosan-25-oate (20)

As shown in FIG. 6, a solution of 18 (315 mg, 0.57 mmol) in CH₂Cl₂ (3 mL) was treated with TFA (0.5 mL) at 0° C. Reaction mixture was stirred at RT for 30 min, then evaporated and codistilled with toluene (10 mL). The resulting syrupy trifluroacetate salt of 19 was kept in vacuo. In the meantime, a mixture of Fmoc-DON (269 mg, 0.68 mmol), HOBt (123 mg, 0.91 mmol), EDC.HCl (175 mg, 0.91 mmol) and DMAP (4 mg, 0.03 mmol) in CH₂Cl₂ (5 mL) was stirred at 0° C. until complete dissolution.

The previously prepared trifluroacetate salt of 19 was dissolved in CH₂Cl₂ (15 mL) and DIPEA (0.11 mL, 0.63 mmol) and added to the mixture, which was then stirred at RT for 2 h. The solvent was evaporated and the residue was subjected to RP flash chromatography (50 g HP C18 Aq column, 30 mL/min, gradient 20%-100% acetonitrile/water in 20 min, detection 265 nm). Yield 335 mg (71%) of lyophilisate.

### Allyl (18S,21S)-21-amino-18-benzyl-25-diazo-17,20,24-trioxo-4,7,10,13-tetraoxa-16,19-diazapentacosanoate (21)

As shown in FIG. 6, a solution of 20 (315 mg, 0.38 mmol) in CH₂Cl₂ (3 mL) was treated with Et₂NH (0.5 mL) and the resulting mixture was stirred at RT for 1 h. The volatiles were evaporated at RT and a residue was subjected to RP flash chromatography (50 g HP C18 Aq column, 30 mL/min, gradient 0%-100% acetonitrile/water in 20 min, detection 220 nm). Yield 172 mg (75%) of lyophilizate (light yellow syrup). The corresponding mass of the product was proved by UPLC-MS. The product was immediately used for the next step.

### Allyl (5S,8S,11S)-11-benzyl-8-(4-diazo-3-oxobutyl)-1-(9H-fluoren-9-yl)-5-isobutyl-3,6,9,12-tetraoxo-2,16,19,22,25-pentaoxa-4,7,10,13-tetraazaoctacosane-28-oate (22)

As shown in FIG. 6, a solution of Fmoc-L-Leu-OH (110 mg, 0.31 mmol) and HBTU (129 mg, 0.34 mmol) in anhydrous CH₂Cl₂ (3 mL) was treated with DIPEA (0.15 mL, 0.85 mmol) at 0° C. and stirred for 10 min. Then a solution of 21 (172 mg, 0.28 mmol) in CH₂Cl₂ (2 mL) was added to the mixture, which was stirred at RT for 2 h. The solution was concentrated and the residue was subjected to RP flash chromatography (50 g HP C18 Aq column, 30 ml/min, gradient 20%-100% acetonitrile/water in 20 min, detection 265 nm). Yield 125 mg (47%) of lyophilisate.

### (5S,8S,11S)-11-Benzyl-8-(4-diazo-3-oxobutyl)-1-(9H-fluoren-9-yl)-5-isobutyl-3,6,9,12-tetraoxo-2,16,19,22,25-pentaoxa-4,7,10,13-tetraazaoctacosane-28-oic acid (Structure VI)

As shown in FIG. 6, a mixture of 22 (108 mg, 0.12 mmol) and dimedone (80 mg, 0.57 mmol) in THF (2 mL) was treated with a solution of Pd(PPh₃)₄ (6.6 mg, 0.006 mmol) in THF (0.5 mL). The mixture was stirred for 1 h at RT, then concentrated and a residue was subjected to RP flash chromatography (50 g HP C18 Aq column, 30 ml/min, gradient 0%-100% acetonitrile/water in 25 min, detection 265 nm). Yield 65 mg (63%) of lyophilisate.

### Allyl 2,2-dimethyl-4-oxo-3,8,11,14-tetraoxa-5-azaheptadecan-17-oate (24)

As shown in FIG. 7, a mixture of 1-(t-Butyloxycarbonyl)amino-3,6,9-trioxadodecan-12-oic acid 23 (707 mg, 2.2 mmol) and Cs₂CO₃ (788 mg, 2.42 mmol) in MeCN (10 mL) was treated with allyl bromide (209 mL, 2.42 mmol). The mixture was heated to 50° C. for 1 h then cooled down, diluted with EtOAc (10 mL) and precipitated salts were filtered off. The residue was chromatographed on a silica gel column in 5% MeOH/CHCl₃. Yield 770 mg (97%) of syrup.

### Allyl (S)-6-benzyl-2,2-dimethyl-4,7-dioxo-3,11,14,17-tetraoxa-5,8-diazaicosan-20-oate (26)

As shown in FIG. 7, a solution of 24 (610 mg, 1.69 mmol) in CH₂Cl₂ (6 mL) was treated with TFA (3 mL) at 0° C. Reaction mixture was stirred at RT for 30 min, then evaporated and codistilled with toluene (10 mL). The resulting syrupy trifluroacetate salt of 25 was kept in vacuo. In the meantime, a solution of Boc-L-Phe (447 mg, 1.69 mmol) and HATU (706 mg, 1.86 mmol) in anhydrous DMF (2 mL) was treated with DIPEA (1.2 mL, 6.75 mmol) at 0° C. and stirred for 10 min. The previously prepared trifluroacetate salt of 25 was dissolved in anhydrous DMF (5 mL) and added to the mixture, which was then stirred at RT for 3 h. The solution was concentrated and the residue was subjected to RP flash chromatography (100 g HP C18 Aq column, 60 mL/min, gradient 0%-100% acetonitrile/water in 25 min, detection 220 nm). Yield 820 mg (95%) of syrup.

### Allyl (5S,8S)-8-benzyl-5-(4-diazo-3-oxobutyl)-1-(9H-fluoren-9-yl)-3,6,9-trioxo-2,13,16,19-tetraoxa-4,7,10-triazadocosan-22-oate (28)

As shown in FIG. 7, a solution of 26 (790 mg, 1.55 mmol) in CH₂Cl₂ (10 mL) was treated with TFA (5 mL) at 0° C. Reaction mixture was stirred at RT for 30 min, then evaporated and codistilled with toluene (15 mL). The resulting syrupy trifluroacetate salt of 27 was kept in vacuo. In the meantime, a mixture of Fmoc-DON (764 mg, 1.94 mmol), HOBt (393 mg, 2.91 mmol), EDC.HCl (558 mg, 2.91 mmol) and DMAP (4 mg, 0.03 mmol) in CH₂Cl₂ (10 mL) was stirred at 0° C. until complete dissolution. The previously prepared trifluroacetate salt of 27 was dissolved in CH₂Cl₂ (15 mL) and DIPEA (540 mL, 3.10 mmol) and added to the mixture, which was then stirred at RT for 2 h. The solvent was evaporated and the residue was subjected to RP flash chromatography (100 g HP C18 Aq column, 60 m/min, gradient 20%-100% acetonitrile/water in 20 min, detection 265 nm). Yield 727 mg (60%) of lyophilisate.

### Allyl (5S,8S,11S)-11-benzyl-8-(4-diazo-3-oxobutyl)-1-(9H-fluoren-9-yl)-5-isobutyl-3,6,9,12-tetraoxo-2,16,19,22-tetraoxa-4,7,10,13-tetraazapentacosan-25-oate (30)

As shown in FIG. 7, a suspension of 28 (718 mg, 0.916 mmol) in MeCN (5 mL) was treated with Et₂NH (5 mL) and the resulting solution was stirred at RT for 30 min. The solvents were evaporated at RT, the residue (crude 29) was codistilled with MeCN (10 mL) and kept in vacuo briefly. The residue was dissolved in MeCN (10 mL) and treated with a solution of Fmoc-L-Leu-OSu (371 mg, 0.824 mmol) in MeCN (10 mL). The mixture was stirred at RT for 4 h. The reaction was quenched by addition of a solution of NH₂OH·HCl (14 mg) and NaHCO₃ (20 mg) in water (1 mL). The mixture was stirred 40 min at RT, then concentrated and the residue was subjected to RP flash chromatography (100 g HP C18 Aq column, 60 m/min, gradient 20%-100% acetonitrile/water in 20 min, detection 265 nm). Yield 398 mg (48%) of lyophilisate.

### (5S,8S,11S)-11-benzyl-8-(4-diazo-3-oxobutyl)-1-(9H-fluoren-9-yl)-5-isobutyl-3,6,9,12-tetraoxo-2,16,19,22-tetraoxa-4,7,10,13-tetraazapentacosan-25-oic acid (Structure VII)

As shown in FIG. 7, a mixture of 30 (359 mg, 0.40 mmol) and dimedone (168 mg, 1.2 mmol) in THF (6 mL) was treated with a solution of Pd(PPh₃)₄ (18 mg, 0.016 mmol) in THF (0.5 mL). The mixture was stirred 1 h at RT, then concentrated and the residue was subjected to RP flash chromatography (100 g HP C18 Aq column, 60 m/min, gradient 0%-100% acetonitrile/water in 25 min, detection 265 nm). Yield 255 mg (74%) of lyophilisate.

### Allyl-L-pyroglutamate (31)

Compound 31 as shown in FIG. 9 was prepared according to the literature (Gang, Fang-li, et al., Bioorganic and Medicinal Chemistry, 2018, vol. 26, 16, p. 4644-4649)

### 1-((9H-fluoren-9-yl)methyl) 2-allyl (S)-5-oxopyrrolidine-1,2-dicarboxylate (32)

As shown in FIG. 9, Allyl-L-pyroglutamate 31 (11.64 g, 68.79 mmol) was dissolved in anhydrous THF (500 mL) and the solution was cooled to −78° C. A solution of LiHMDS (1M in THF, 66 mL, 66 mmol) was added. The resulting mixture was stirred 15 min at −78° C. and then added via cannula to a solution of Fmoc-Cl (53.4 g, 206.4 mmol) in THF (300 mL). Reaction mixture was stirred 3 h at −78° C. then allowed to reach RT and left overnight. Reaction was quenched with saturated aqueous solution of NH₄Cl (400 mL). Organic layer was separated and aqueous portion was extracted with EtOAc (2×300 mL). Combined organic extracts were dried (MgSO₄) and most of the solvents were evaporated. Liquid residue was diluted with Et₂O (300 mL) and the solution was allowed to crystallize in a fridge overnight. The crystals were filtered off, washed with Et₂O and the filtrate was concentrated. The syrupy residue was chromatographed on a silica gel column in 25-50% EtOAc/hexane. Yield 26.22 g (97%) of syrup.

### Allyl (S)-2-((((9H-fluoren-9-yl)methoxy)carbonyl)amino)-6-diazo-5-oxohexanoate (33)

As shown in FIG. 9, a solution of trimethylsilyldiazomethane (2 M solution in hexane, 41.3 mL, 82.6 mmol) in THF (150 mL) was cooled to −78° C. and treated with n-BuLi (2.5 M in hexane, 33.8 mL, 84.6 mmol). The mixture was stirred 30 min at −78° C. and then added via cannula to a cooled (−78° C.) solution of 32 (26.00 g, 66.08 mmol) in THF (450 mL). The solution was stirred additional 30 min at −78° C. and then quenched with aqueous solution of sat. NH₄Cl (300 mL). The mixture was allowed to reach RT, aqueous layer was separated and extracted with EtOAc (2×200 mL). Combined organic portions were dried (MgSO₄) and concentrated. The residue was purified on a silica gel column in 40-50% EtOAc/hexane. Crystallisation from EtOAc/hexane gave 20.48 g (71%).

### (S)-2-((((9H-fluoren-9-yl)methoxy)carbonyl)amino)-6-diazo-5-oxohexanoic acid (34)

As shown in FIG. 9, a solution of allyl ester 33 (20.20 g, 46.60 mmol) and dimedone (19.6 g, 139.8 mmol) in THF (230 mL) was treated with a solution of Pd(PPh₃)₄ (2.15 g, 1.86 mmol) in THF (70 mL). The mixture was stirred 90 min, then concentrated and the residue was chromatographed on a silica gel column in 10-20% MeOH/CH₂Cl₂ giving 10.95 g (60%).

### 1-((9H-fluoren-9-yl)methyl) 2-octyl (S)-5-oxopyrrolidine-1,2-dicarboxylate (36)

As shown in FIG. 9, to a solution of (S)-octyl 5-oxopyrrolidine-2-carboxylate 35 (10 g, 41.44 mmol) in anhydrous THF (200 mL) was added LiHMDS (39.37 mL, 39.37 mmol, 1 M in THF) dropwise at −78° C. under inert atmosphere and the mixture was stirred for 20 min. The resultant yellow mixture was transferred via cannula to a solution of Fmoc-Cl (12.86 g, 49.73 mmol) in dry THF (150 mL) at −78° C. The reaction mixture was stirred at −78° C. for 2 h and at slowly warm up to rt overnight. The reaction was quenched with saturated NH₄Cl (100 mL) and water (50 mL). The aqueous phase was extracted with ethyl acetate (3×100 mL) and the combined organic layers were washed with brine (200 mL) and dried over anhydrous MgSO₄. The solvent was removed under reduced pressure. The residue was chromatographed on silica gel in 20-100% EtOAc/hexane to give 18.23 g (95% yield) of compound 36 as a colorless foam.

### Octyl (S)-2-((((9H-fluoren-9-yl)methoxy)carbonyl)amino)-6-diazo-5-oxohexanoate (37)

As shown in FIG. 9, a solution of trimethylsilyl diazomethane (23.6 mL, 47.2 mmol, 2 M in diethyl ether) was dissolved in anhydrous THF (210 mL) under argon and cooled to −98° C. (Dry ice acetone bath and liquid N₂). A solution of n-BuLi (19.35 mL, 48.38 mmol, 2.5 M in hexanes) was added dropwise, and the solution was stirred at −98° C. for 30 min. The resultant mixture was transferred via cannula to a solution of compound 36 (18.23 g, 39.33 mmol) in dry THF (360 mL) at −98° C. (Dry ice acetone bath and liquid N₂). The reaction mixture was stirred for 30 min at −98° C. and then slowly warmed to −78° C. and quenched with saturated NH₄Cl (200 mE). The aqueous phase was extracted with ethyl acetate (3×100 mL) and the combined organic layers were washed with brine (200 mL) and dried over anhydrous MgSO₄. The solvent was removed under reduced pressure. The residue was chromatographed on silica gel in 5% CHCl₃/acetone to give 17.2 g (86% yield) of compound 37 as a light yellow solid.

### Octyl (S)-2-amino-6-diazo-5-oxohexanoate (38)

As shown in FIG. 9, to a solution of compound 37 (5.06 g, 10 mmol) in anhydrous DMF (20 mL) was added piperidine (0.94 g, 11 mmol) at rt under inert atmosphere. The mixture was stirred for 20 min and quenched with H₂O (80 mL). The aqueous phase was extracted with ethyl acetate (3×100 mL) and the combined organic layers were washed with brine (100 mL) and dried over anhydrous MgSO₄. The solvent was removed under reduced pressure. The residue was chromatographed on silica gel in 0-10% MeOH/DCM containing 0.5% Et₃N to give 2.33 g (82% yield) of compound 38 as a yellow solid, which was immediately used in the subsequent step.

### Octyl (S)-2-((S)-2-((((9H-fluoren-9-yl)methoxy)carbonyl)amino)-6-diazo-5-oxohexanamido)-6-diazo-5-oxohexanoate (39)

To a solution of compound 34 (2.75 g, 6.99 mmol) and 38 (2.38 g, 8.39 mmol) in anhydrous DCM (40 mL) was added N, N-diisopropylethylamine (DIPEA, 1.81 g, 13.98 mmol) and the mixture was cooled to 0° C. under inert atmosphere. HATU (2.93 g, 7.69 mmol) was added in one portion and the mixture was stirred at 0° C. for 1 h and quenched with H₂O (80 mL). The aqueous phase was extracted with ethyl acetate (3×100 mL) and the combined organic layers were washed with brine (100 mL) and dried over anhydrous MgSO₄. The solvent was removed under reduced pressure. The residue was chromatographed on silica gel in 0-10% MeOH/DCM to give 4.0 g (87% yield) of compound 39 as a yellow solid which was immediately used in the subsequent step.

### Octyl (S)-2-((S)-2-amino-6-diazo-5-oxohexanamido)-6-diazo-5-oxohexanoate (40)

To a solution of compound 39 (4.0 g, 6.07 mmol) in anhydrous DMF (20 mL) was added piperidine (0.57 g, 6.68 mmol) at rt under inert atmosphere. The reaction was carefully monitored and quenched with H₂O (60 mL) upon the disappearance of compound 39 (approximately 20 min). The aqueous phase was extracted with ethyl acetate (3×80 mL) and the combined organic layers were washed with brine (100 mL) and dried over anhydrous MgSO₄. The solvent was removed under reduced pressure. The residue was chromatographed on silica gel in 0-10% MeOH/DCM containing 0.5% Et₃N to give 1.8 g (68% yield) of compound 40 as a yellow solid which was immediately used in the subsequent step.

### 5-allyl 1-octyl (((9H-fluoren-9-yl)methoxy)carbonyl)-L-glutamate (42)

To a solution of 41 (3.80 g, 9.29 mmol) in anhydrous DCM (100 mL) was added 1-octanol (1.21 g, 9.29 mmol) and the mixture was stirred at 0° C. DCC (1.92 g, 9.29 mmol) was added followed by catalytic amount of DMAP under inert atmosphere. Reaction mixture was stirred overnight at rt. Precipitated DCU was filtered off and solvent was removed under reduced pressure. The residue was re-dissolved in EtOAc (100 mL) and the remaining precipitate of DCU was filtered off. The solvent was removed under reduced pressure. The residue was chromatographed on a silica gel column in 10-100% EtOAc/hexane to give 3.29 g (68% yield) of compound 42 as a white solid.

### (S)-4-((((9H-fluoren-9-yl)methoxy)carbonyl)amino)-5-(octyloxy)-5-oxopentanoic acid (43)

To a solution of 42 (3.29 g, 6.31 mmol) and PhSiH₃ (1.37 mg, 12.62 mmol) in anhydrous DCM (100 mL) was added Pd(PPh₃)₄ (219 mg, 0.19 mmol) at 0° C. under inert atmosphere and the mixture was slowly warm up to rt and stirred at for 40 min. A few drops of water were then added and the volatiles were removed under reduced pressure. The residue was chromatographed on a silica gel column in 10-100% EtOAc containing 0.5% AcOH/hexane to give 2.41 g (77% yield) of compound 43 as a white solid.

### Octyl (5S,10S,13S)-10,13-bis(4-diazo-3-oxobutyl)-1-(9H-fluoren-9-yl)-5-((octyloxy)carbonyl)-3,8,11-trioxo-2-oxa-4,9,12-triazatetradecan-14-oate (44)

To a solution of compound 40 (1.80 g, 4.12 mmol) and 43 (1.99 g, 4.12 mmol) in anhydrous DCM (40 mL) was added N, N-diisopropylethylamine (DIPEA, 1.07 g, 8.25 mmol) and the mixture was cooled to 0° C. under inert atmosphere. HATU (1.73 g, 4.54 mmol) was added in one portion and the mixture was stirred at 0° C. for 40 min and quenched with H₂O (80 mL). The aqueous phase was extracted with ethyl acetate (3×100 mL) and the combined organic layers were washed with brine (100 mL) and dried over anhydrous MgSO₄. The solvent was removed under reduced pressure. The residue was chromatographed on silica gel in 0-10% MeOH/DCM to give 3.2 g (86% yield) of compound 44 as a yellow solid.

### Octyl (S)-2-((S)-2-((S)-4-amino-5-(octyloxy)-5-oxopentanamido)-6-diazo-5-oxohexanamido)-6-diazo-5-oxohexanoate (Structure IX)

To a solution of compound 44 (1.5 g, 1.67 mmol) in DMF (15 mL) was added piperidine (0.16 g, 1.83 mmol) at rt under inert atmosphere. The mixture was stirred for 40 min and quenched with H₂O (60 mL). The aqueous phase was extracted with ethyl acetate (3×80 mL) and the combined organic layers were washed with brine (100 mL) and dried over anhydrous MgSO₄. The solvent was removed under reduced pressure. The residue was chromatographed on silica gel in 0-10% MeOH/CHCl₃ containing 0.5% Et₃N to give 0.81 g (72% yield) of compound IX as a light yellow solid.

### Octyl (16S,21S,24S)-21,24-bis(4-diazo-3-oxobutyl)-1-hydroxy-16-((octyloxy)carbonyl)-14,19,22-trioxo-3,6,9,12-tetraoxa-15,20,23-triazapentacosan-25-oate (Structure X)

To a solution of compound IX (300 mg, 0.44 mmol), hydroxy-PEG₄-CH₂CO₂H (122.8 mg, 0.49 mmol) and N, N-diisopropylethylamine (DIPEA, 200 mg, 1.55 mmol) in THF (5 mL) was added 1-hydroxybenzotriazole hydrate (HOBt·H₂O, 102 mg, 0.66 mmol) and N-(3-dimethylaminopropyl)-N′-ethylcarbodiimide hydrochloride (EDC hydrochloride, 119 mg, 0.62 mmol) was at rt under inert atmosphere. The mixture was stirred for 3 h and quenched with saturated NaHCO₃ (10 mL). The aqueous phase was extracted with ethyl acetate (3×20 mL) and the combined organic layers were washed with brine (10 mL) and dried over anhydrous MgSO₄. The solvent was removed under reduced pressure. The residue was purified using reverse phase preparative HPLC (40% acetonitrile/60% water followed by an increase to 100% acetonitrile over 60 min and wash with 100% acetonitrile over 10 min; flow rate 15 mL/min) to give 142 mg (35% yield) of compound X as a light-yellow sticky solid.

### 5-allyl 1-(2-(2-(2-(2-hydroxyethoxy)ethoxy)ethoxy)ethyl) (((9H-fluoren-9-yl)methoxy)carbonyl)-L-glutamate (45)

To a solution of 41 (1.0 g, 2.44 mmol) in anhydrous DCM (20 mL) was added 2,2′-((oxybis(ethane-2,1-diyl))bis(oxy))bis(ethan-1-ol) (1.42 g, 7.33 mmol) and the mixture was stirred at 0° C. DCC (504 mg, 2.44 mmol) was added followed by catalytic amount of DMAP (30 mg, 0.24 mmol) under inert atmosphere. Reaction mixture was stirred 4 h at 0° C. Precipitated DCU was filtered off and solvent was removed under reduced pressure. The solvent was removed under reduced pressure. The residue was chromatographed on a silica gel column in 20-100% EtOAc/hexane to give 0.8 g (56% yield) of compound 45 as a colorless oil. ¹H NMR (500 MHz, CDCl₃): δ 2.00-2.07 (m, 1H), 2.22-2.29 (m, 1H), 2.39-2.51 (m, 2H), 3.58-3.71 (m, 14H), 4.22 (t, J=6.5 Hz, 1H), 4.28-4.35 (m, 2H), 4.40-4.47 (m, 3H), 4.58 (d, J=6.0 Hz, 2H), 5.23 (d, J=10.5 Hz, 1H), 5.33-5.29 (m, 2H), 5.79 (d, J=8.5 Hz, 1H), 5.95-5.87 (m, 1H), 7.30-7.33 (m, 2H), 7.38-7.41 (m, 2H), 7.59-7.62 (m, 2H), 7.76 (d, J=7.5 Hz, 2H). ESI MS: 608.3 ([M+Na]+).

### (S)-14-((((9H-fluoren-9-yl)methoxy)carbonyl)amino)-1-hydroxy-13-oxo-3,6,9,12-tetraoxaheptadecane-17-oic acid (46)

To a solution of 45 (0.8 g, 1.37 mmol) and PhSiH₃ (296 mg, 2.73 mmol) in anhydrous DCM (15 mL) was added Pd(PPh₃)₄ (40 mg, 0.035 mmol) at 0° C. under inert atmosphere and the mixture was slowly warm up to rt and stirred for 40 min. A few drops of water were then added and the volatiles were removed under reduced pressure. The residue was chromatographed on a silica gel column in 10-40% MeOH/DCM containing 0.5% AcOH to give 0.49 g (66% yield) of compound 46 as a white solid. ESI MS: 568.3 ([M+Na]+).

### Octyl (5S,10S,13S)-10,13-bis(4-diazo-3-oxobutyl)-1-(9H-fluoren-9-yl)-5-(13-hydroxy-2,5,8,11-tetraoxatridecanoyl)-3,8,11-trioxo-2-oxa-4,9,12-triazatetradecan-14-oate (XI)

To a solution of compound 40 (200 mg, 0.46 mmol) and 46 (250 mg, 0.46 mmol) in anhydrous DCM (15 mL) was added N, N-diisopropylethylamine (DIPEA, 118 mg, 0.92 mmol) and the mixture was cooled to 0° C. under inert atmosphere. HATU (192 mg, 0.50 mmol) was added in one portion and the mixture was stirred at 0° C. for 2 h and quenched with H₂O (80 mL). The aqueous phase was extracted with ethyl acetate (3×50 mL) and the combined organic layers were washed with brine (50 mL) and dried over anhydrous MgSO₄. The solvent was removed under reduced pressure. The residue was purified using reverse phase preparative HPLC (20% acetonitrile/80% water followed by an increase to 70% acetonitrile over 40 min and wash with 100% acetonitrile over 10 min; flow rate 15 mL/min) to give 145 mg (33% yield) of compound XI as a light yellow solid. ¹H NMR (500 MHz, CDCl₃): δ 0.86 (t, J=6.5 Hz, 3H), 1.23-1.32 (m, 10H), 1.58-1.63 (m, 2H), 1.91-2.24 (m, 6H), 2.33-2.39 (m, 3H), 2.43-2.51 (m, 4H), 3.43 (brs, 1H), 3.55-3.72 (m, 14H), 4.05-4.11 (m, 2H), 4.19-4.28 (m, 2H), 4.33-4.43 (m, 4H), 4.47-4.51 (m, 1H), 5.35-5.39 (m, 2H), 6.24 (d, J=8.0 Hz, 1H), 7.11 (d, J=7.0 Hz, 1H), 7.29-7.32 (m, 2H), 7.37-7.40 (m, 2H), 7.45 (d, J=8.0 Hz, 1H), 7.59-7.62 (m, 2H), 7.74 (d, J=7.5 Hz, 2H). ¹³C NMR (125 MHz, CDCl₃): δ 14.0, 22.5, 25.7, 26.7, 27.7, 27.9, 28.4, 29.07, 29.09, 31.7, 36.2, 36.4, 47.1, 51.9, 52.5, 53.4, 54.8, 55.1, 61.4, 64.2, 65.7, 66.8, 68.7, 70.1, 70.3, 70.4, 72.5, 119.9, 125.1, 127.0, 127.6, 141.2, 143.7, 143.9, 156.2, 171.3, 171.6, 171.9, 172.3, 193.8, 194.8. ESI MS: 986.4 ([M+Na]+).

### Allyl (S)-5-(4-diazo-3-oxobutyl)-1-(9H-fluoren-9-yl)-3,6-dioxo-2,10,13,16-tetraoxa-4,7-diazanonadecan-19-oate (47)

Fmoc-DON-OH (694 mg, 1.76 mmol, 1.1 equiv.) and HATU (701 mg, 1.84 mmol, 1.15 equiv.) were suspended in anhydrous DCM (10 mL), cooled to 0° C. and DIPEA (1.12 mL, 6.41 mmol, 4.0 equiv.) was added. After 10 min of stirring, the solution of 25 (419 mg, 1.60 mmol, 1.0 equiv.) in anhydrous DCM (5 mL) was added dropwise. The reaction mixture was stirred for 30 min at 0° C. and for 1.5 h at rt. The volatiles were evaporated in vacuo and the residue was purified on silica gel (DCM/MeOH, 20:1). The desired product 47 (1.0 g, 98%) was observed as a yellowish oil. ¹H NMR (401 MHz, CDCl₃): 1.90-2.03 (m, 1H), 2.10-2.16 (m, 1H), 2.37-2.56 (m, 2H), 2.61 (t, J=6.4 Hz, 2H), 3.18 (q, J=7.4 Hz, 2H), 3.58-3.64 (m, 10H), 3.66-3.78 (m, 2H), 4.21 (t, J=7.1 Hz, 2H), 4.31-4.44 (m, 2H), 4.58 (dt, J=5.7, 1.4 Hz, 2H), 5.15-5.37 (m, 2H), 5.80-5.97 (m, 2H), 6.86-6.94 (m, 1H), 7.27-7.44 (m, 5H), 7.60 (d, J=7.6 Hz, 2H), 7.76 (d, J=7.5 Hz, 2H). UPLC MS: [M+Na]+(C₃₃H₄₀N₄O₉Na): 659.448.

### Allyl (S)-15-amino-19-diazo-14,18-dioxo-4,7,10-trioxa-13-azanonadecanoate (48)

Compound 47 (1.0 g, 1.57 mmol, 1.0 equiv.) was dissolved in anhydrous DCM (16 mL), Et₂NH (1.63 mL, 15.7 mmol, 10.0 equiv.) was added and the reaction mixture was stirred for 2 h at rt. The volatiles were evaporated and the residue 48 was used to the further reaction without any purification.

### Allyl (5S,8S)-5,8-bis(4-diazo-3-oxobutyl)-1-(9H-fluoren-9-yl)-3,6,9-trioxo-2,13,16,19-tetraoxa-4,7,10-triazadocosan-22-oate (49)

Fmoc-DON-OH (680 mg, 1.73 mmol, 1.1 equiv.) and HATU (687 mg, 1.81 mmol, 1.15 equiv.) were suspended in anhydrous DM (15 mL), cooled to 0° C. and DIPEA (1.09 mL, 6.29 mmol, 4.0 equiv.) was added. After 10 min of stirring, the solution of 48 (651 mg, 1.57 mmol, 1.0 equiv.) in anhydrous DCM (7 mL) was added dropwise and the reaction mixture was stirred for 30 min at 0° C. and for 1.5 h at rt. The volatiles were evaporated in vacuo and the residue was purified on flash chromatography (gradient 5% to 100% of acetonitrile in H₂O, 25 min). The desired product 49 (525 mg, 42%) was observed as a yellowish solid. ¹H NMR (401 MHz, DMSO-d₆): 1.68-1.93 (m, 4H), 2.23-2.40 (m, 4H), 2.57 (t, J=6.2 Hz, 2H), 3.39 (t, J=5.9 Hz, 2H), 3.43-3.52 (m, 10H), 3.63 (t, J=6.2 Hz, 2H), 3.95-4.03 (m, 1H), 4.16-4.36 (m, 4H), 4.55 (dt, J=5.4, 1.6 Hz, 2H), 5.16-5.32 (m, 2H), 5.83-6.07 (m, 3H), 7.29-7.46 (m, 4H), 7.56 (d, J=7.9 Hz, 1H), 7.68-7.77 (m, 2H), 7.89 (d, J=7.5 Hz, 2H), 7.92-8.02 (m, 2H). UPLC MS: [M+Na]+(C₃₉H₄₇N₇O₁₁Na): 812.916.

### Allyl (15S,18S)-18-amino-22-diazo-15-(4-diazo-3-oxobutyl)-14,17,21-trioxo-4,7,10-trioxa-13,16-diazadocosanoate (50)

Compound 49 (525 mg, 0.665 mmol, 1.0 equiv.) was dissolved in anhydrous DMF (5 mL) and Et₂NH (688 μL, 6.65 mmol, 10.0 equiv.) was added. The reaction mixture was stirred for 2 h at rt. The volatiles were evaporated in vacuo and the residue 50 was used to the further reaction without any purification.

### Allyl (6S,9S)-6,9-bis(4-diazo-3-oxobutyl)-2-methyl-4,7,10-trioxo-14,17,20-trioxa-2,5,8,11-tetraazatricosan-23-oate (51)

Compound 50 (377 mg, 0.665 mmol, 1.0 equiv.) and 2,5-dioxopyrrolidin-1-yl dimethylglycinate (266 mg, 1.33 mmol, 2.0 equiv.) were suspended in anhydrous DMF (5 mL) and the suspension was stirred for 2 h at rt. The volatiles were evaporated in vacuo and the residue was purified on silica gel (DCM/MeOH, 10:1). The desired product 51 (228 mg, 53%) was observed as a yellowish oil. ¹H NMR (401 MHz, CDCl₃): 1.95-2.21 (m, 4H), 2.32 (s, 6H), 2.35-2.58 (m, 4H), 2.91-3.09 (m, 2H), 3.39-3.47 (m, 2H), 3.52-3.58 (m, 2H), 3.58-3.66 (m, 10H), 3.77 (t, J=6.4 Hz, 2H), 4.30-4.42 (m, 2H), 4.59 (dt, J=5.6, 1.5 Hz, 2H), 5.17-5.44 (m, 4H), 5.91 (ddt, J=17.2, 10.4, 5.7 Hz, 1H), 6.85-6.92 (m, 1H), 7.48-7.59 (m, 1H), 7.80 (d, J=7.2 Hz, 1H). UPLC MS: [M+H]⁺ (C₂₈H₄₅N₈O₁₀): 653.155.

### (6S,9S)-6,9-bis(4-diazo-3-oxobutyl)-2-methyl-4,7,10-trioxo-14,17,20-trioxa-2,5,8,11-tetraazatricosan-23-oic acid (XII) (KNM472)

Compound 51 (176 mg, 0.269 mmol, 1.0 equiv.) and dimedone (113 mg, 0.809 mg, 3.0 equiv.) were dissolved in anhydrous THF (3 mL), Pd(PPh₃)₄ (12 mg, 0.011 mmol, 0.04 equiv.) was added and the reaction mixture was stirred for 1 h at rt. The volatiles were evaporated in vacuo and the residue was purified on flash chromatography (gradient 5% to 100% of acetonitrile in H₂O, 25 min) and on silica gel (DCM/MeOH, 10:1+1% Et₃N). The desired product XII (51 mg, 31%) was observed as a yellowish oil. ¹H NMR (401 MHz, DMSO-d₆): 1.68-1.99 (m, 4H), 2.21 (s, 6H), 2.25-2.35 (m, 4H), 2.52-2.60 (m, 2H), 2.81-2.93 (m, 2H), 3.33-3.45 (m, 2H), 3.45-3.53 (m, 10H), 3.59 (t, J=6.4 Hz, 2H), 4.15-4.23 (m, 1H), 4.26-4.35 (m, 1H), 6.02 (bs, 2H), 7.81 (d, J=8.2 Hz, 1H), 7.98 (t, J=5.7 Hz, 1H), 8.14 (d, J=7.9 Hz, 1H). UPLC MS: [M+H]⁺ (C₂₅H₄₁N₈O₁₀): 613.048.

### Liver Homogenate Studies

Liver or EL4 lymphoma tumor tissues were washed and diluted 10-fold in 0.1 M potassium phosphate buffer and homogenized using a probe sonicator. To evaluate the stability of the analogs 1 mL aliquots were made of each matrix and the analogs were spiked to a final assay concentration of 20 μm. Spiked samples were incubated in an orbital shaker at 37° C. for 1 h, following which reactions were quenched in triplicate with three volumes of acetonitrile containing the internal standard (IS; losartan: 0.5 μm). The samples were vortex-mixed for 30 s and centrifuged at 10 000 g for 10 min at 4° C. Fifty microliters of the supernatant was diluted with 50 μL of water and transferred to a 250 μL polypropylene vial sealed with a Teflon cap. Compound disappearance was monitored over time using liquid chromatography tandem mass spectrometry (LC-MS/MS).

### DON Release Assay

DON release was evaluated in liver homogenate or tumor homogenate using previously described methods. Briefly, supernatants were dried at 45° C. under vacuum for 1 h. To each tube, 50 μL of 0.2 M sodium bicarbonate buffer (pH 9.0) and 100 μL of 10 mM dabsyl chloride was added. After vortex-mixing, samples were incubated at 60° C. for 15 min to derivatize, followed by centrifugation at 16 000 g for 5 min at 4° C. One hundred microliters of the supernatant was transferred to a 96-well plate, diluted with 400 μL of water and injected onto LC-MS/MS. DON was analyzed on a Dionex ultra high-performance LC system coupled with a Q Exactive Focus orbitrap mass spectrometer (Thermo Fisher Scientific Inc., Waltham, MA). Separation was achieved at 35° C. using an Agilent Eclipse Plus column (100×2.1 mm2, i.d.) packed with a 1.8 μm C18 stationary phase. The mobile phase included 0.1% formic acid in water and 0.1% formic acid in acetonitrile. Pumps were operated at a flow rate of 0.3 mL/min for 3.5 min using gradient elution. The mass spectrometer controlled by Xcalibur software 4.0.27.13 (Thermo Scientific) was operated with a heated ESI ion source in positive ionization mode. Quantification was performed in parallel-reaction monitoring mode.

### Results

TABLE 1
Dendrimer Conjugate precursors synthesized and their
metabolic stability

| | Glutamine Antagonists Synthesized for Dendrimer Conjugation | | |
|---|---|---|---|
| | | Liver | Liver |
| | | homo- | homo- |
| | | genate | genate |
| | | stability | DON |
| | | (% | release (% |
| | | remaining | liberated |
| Com- | | at 1 h | at 1 h |
| pound | | incubation) | incubation) |
| 1 | 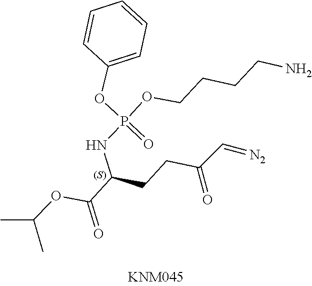 | 32 | <5% |
| II | 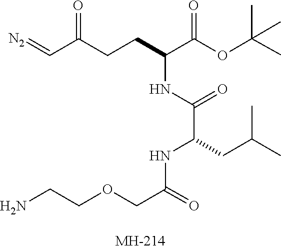 | 94 | <5% |
| III | 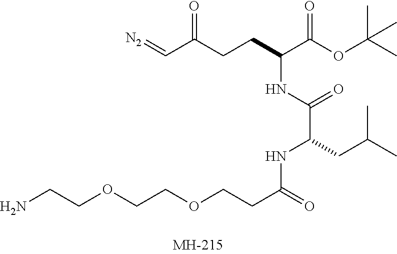 | 79 | <5% |
| IV | 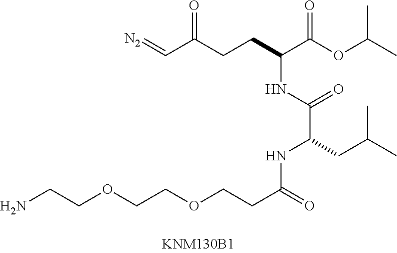 | 0 | <5% |
| V | 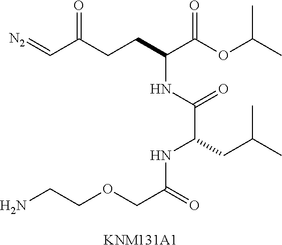 | 17 | <5% |
| VI | 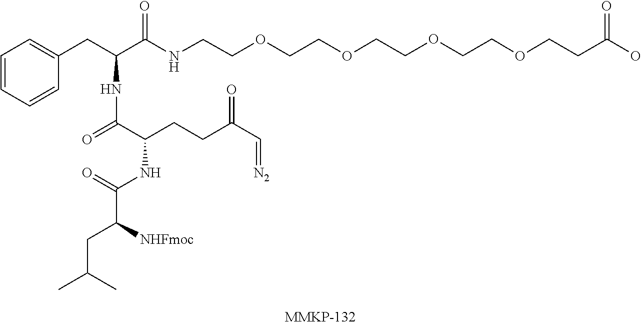 | 12 | ~30% |
| VII | 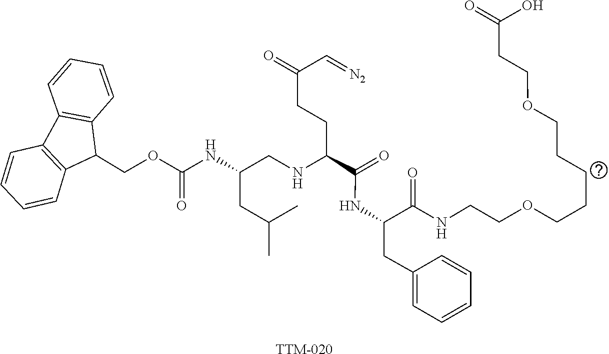 | 0 | ~30% |
| VIII | 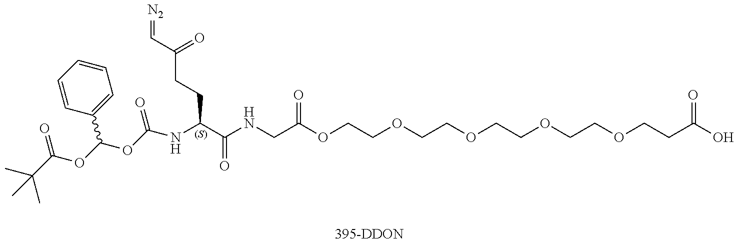 | NA | NA |
| IX | 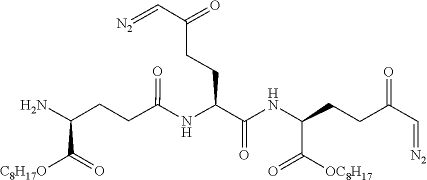 | | |
| X | 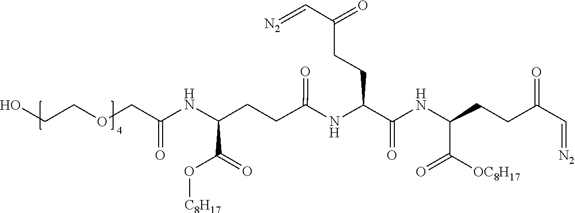 | | |
| XI | 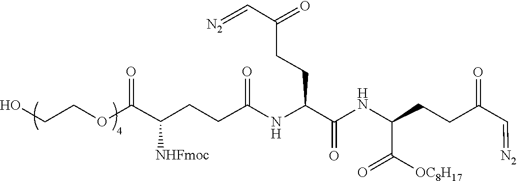 | | |
| XII | 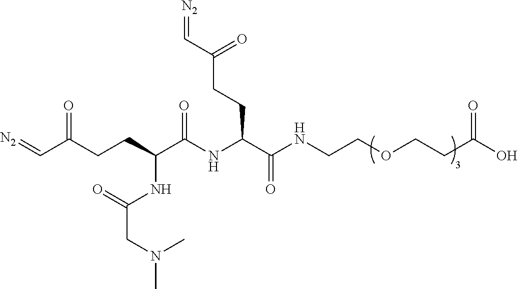 | | |

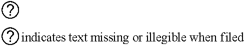

### Example 2: Synthesis of Glutamine-Antagonist Dendrimer Conjugates with Various Linkers

All of the glutamine antagonists were synthesized with linkers amenable for conjugation chemistry on dendrimer surface.

For conjugation, the hydroxyl groups on dendrimer surface were partially modified to attach linkers having reactive functional groups complementary to the groups on the antagonists-linkers (Figure. 9). The antagonists were conjugated on the surface of modified dendrimers using activated acid-amine coupling reaction in the presence of N-hydroxysuccinimide (NHS) and N-(3-dimethyl aminopropyl)-N′-ethylcarbodiimide hydrochloride (EDC). The final conjugates were purified by dialysis to remove the solvent and small molecular weight impurities. The intermediates and the final dendrimer conjugates were characterized using ¹H-NMR, ¹³C-NMR, HPLC, and MALDI-TOF/MS analyses. The conjugates were synthesized with high purity (>95%). The drug loading of the conjugates was calculated via ¹H-NMR using proton integration method by comparing the integration of drug protons to internal amide protons of the dendrimer. The conjugates had a drug loading of ˜5% w/w. The dendrimer-drug conjugates were water soluble with solubility ranging from 50-100 mg/mL.

Following are the detailed synthesis protocols for dendrimer-glutamine antagonist conjugate presented in the FIG. 9:

Synthesis of dendrimer-acid: To a stirring solution of D-OH (1 g, 0.07 mmoles) in DMF (20 mL), N, N diisopropylethylamine (DIPEA; 0.5 mL) was added, followed by the addition of glutaric anhydride (95.8 mg, 0.840 mmoles). The reaction mixture was stirred at room temperature (RT, approximately 25° C.) for 24 hours. The reaction mixture was then diluted with DMF and dialyzed using 1 k Da dialysis membrane against DMF for 12 hours followed by the water dialysis for additional 12 hours. The solvents were replaced every 3-4 hours during dialysis process. The aqueous solution was lyophilized to obtain D-acid as white hygroscopic solid. Yield: 88% ¹H NMR: (500 MHz, DMSO) δ 8.27-7.73 (m, D-inter amide H), 4.07 (t, D-ester —CH₂), 3.54-3.26 (m, D-CH₂), 3.25-3.00 (m, D-CH₂), 2.90-2.60 (m, D-CH₂ and linker CH₂), 2.53-2.31 (m, D-CH₂ and linker CH₂), 2.26 (m, D-CH₂), and 1.85-1.72 (m, linker —CH₂).

Synthesis of dendrimer-glutamine antagonist conjugate: To a stirring solution of D-acid (200 mg, 0.013 mmoles) in DMF (5 mL), DIPEA (0.1 mL) was added, followed by the addition of NHS (22 mg, 0.195 mmoles) and EDC (37.2 mg, 0.195 mmoles). The reaction mixture was stirred for 30 minutes at room temperature. This was followed by the addition of glutamine-antagonist (71.5 mg, 0.156 mmoles). The reaction mixture was stirred at room temperature for 24 hours. The reaction mixture was then diluted with DMF and dialyzed using 1 kDa dialysis membrane against DMF for 12 hours followed by the water dialysis for additional 12 hours. The solvents were replaced every 3-4 hours during dialysis process. The aqueous solution was lyophilized to obtain dendrimer conjugate as white hygroscopic solid. Yield: 72% ¹H NMR (500 MHz, DMSO) δ 8.29 (d, drug-H), 8.16-7.69 (m, drug-H and D-internal amide H), 6.04 (drug-H), 4.87 (m, drug-H), 4.34 (m, drug-H), 4.15 (m, drug-H), 4.01 (t, D-ester —CH₂), 3.59 (m, drug H), 3.52-2.99 (D-CH₂ and drug H), 2.66 (m, D-CH₂ and drug H), 2.46-2.04 (m, D-CH₂ and drug H), 2.01-1.23 (m, drug H and linker-CH₂), 1.17 (d, drug H), and 0.87 (dd, drug H).

HPLC: Retention time: Retention time: 11.028 minutes; Purity: 99%.

### Results

Some exemplary dendrimer and glutamine antagonist conjugates are shown in Table 2 below.

TABLE 2
Exemplary dendrimer and glutamine antagonist conjugates

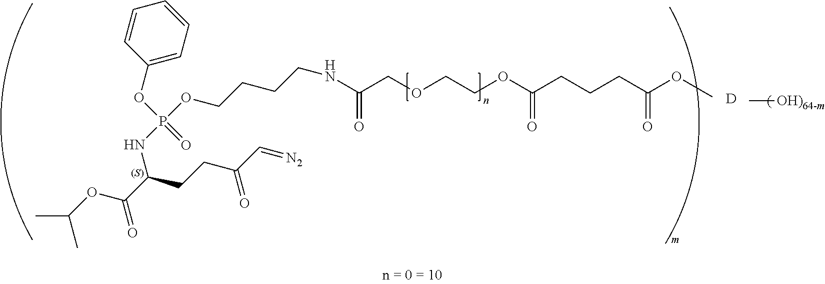

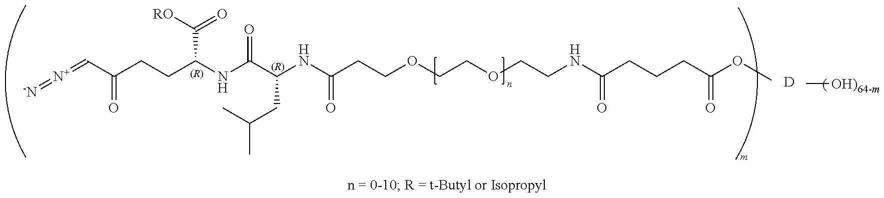

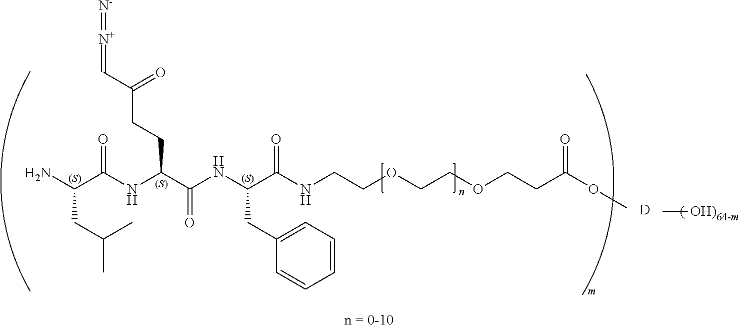

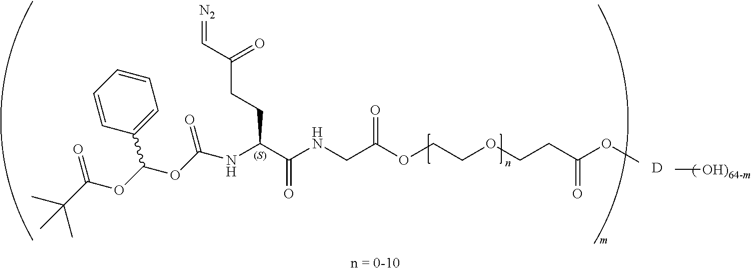

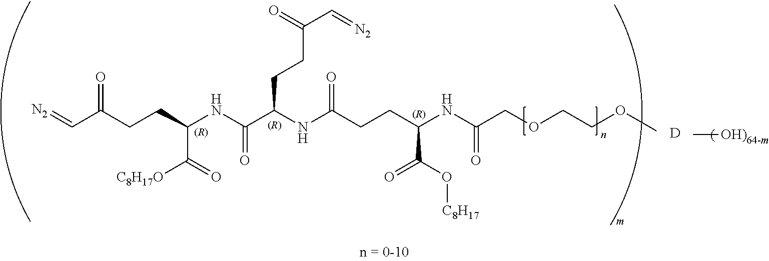

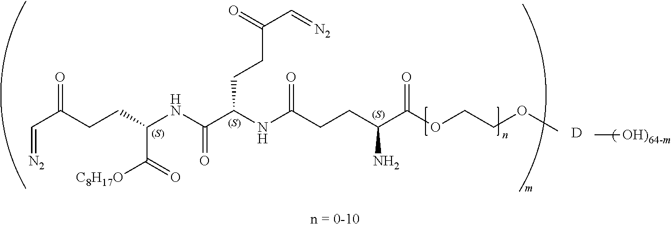

### Example 3: Glutamine Antagonist Dendrimer Conjugate TTM020 (Structure VII) Shows Brain Target Engagement in a Murine Model of Neuroinflammation

### Methods

### Animals and Drug Administration

Mice were injected intracranially with LPS (2.5 mg/kg) into their right striatum. One hour later Dendrimer-TTM020 or empty dendrimer was administered IP at a dose of 2 mg/kg DON equivalence. Twelve and 72 hours later mice were sacrificed and right hemisphere was removed and CD11b cells were extracted as described below. Glutaminase activity assessment was carried out as described below.

### Isolation of Brain CD11b+ Cells

Mice were sacrificed by decapitation and right striatum immediately removed. CD11b+ cells were isolated. In brief, the brain tissue was minced in HBSS (cat #55021C, Sigma-Aldrich, St. Louis, MO, USA) and dissociated with the neural tissue dissociation kits (P) (cat #130-092-628, MACS Militenyi Biotec, Auburn, CA) according to manufacturer instructions. After passing through a 70 μm cell strainer, resulting homogenates were centrifuged at 300×g for 10 min. Supernatants were removed and cell pellets were resuspended, and myelin was removed by Myelin Removal Beads II (cat #130-096-733, MACS Militenyi Biotec, Auburn, CA) according to the manufacturer instructions. Myelin-removed cell pellets were resuspended and incubated with CD11b MicroBeads (cat #130-093-634, MACS Militenyi Biotec, Auburn, CA) for 15 min, loaded on LS columns and separated on a quadroMACS magnet. CD11b+ cells were flushed out from the LS columns, then washed and resuspended in sterile HBSS (cat #55037C, Sigma-Aldrich, St. Louis, MO). The number of viable cells was determined using a hemacytometer and 0.1% trypan blue staining. Each brain extraction yielded 5×10⁵ viable CD11b+ cells. It has been demonstrated that CD11b+ cells isolated from brain homogenates through this antibody-coupled microbeads method are microglia-enriched populations (>95% of isolated cells).

### Glutaminase Activity

Glutaminase activity measurement in CD11b+ cells was conducted next. Briefly, cells were lysed by sonication in ice-cold potassium phosphate buffer (45 mM, pH 8.2) containing protease inhibitors (cat #04693116001, Roche). For CD11b+ and non-CD11b+ cells isolated from prefrontal cortex, hippocampus, and cerebellum, lysates were incubated with [³H]-glutamine (0.09 μM, 2.73 ρCi) for 180 min at room temperature and the reactions carried out in 50 μl reaction volumes in a 96-well microplate. The reaction was then terminated by addition of imidazole buffer (20 mM, pH 7). 96-well spin columns packed with strong anion ion-exchange resin (cat #140-1251, Bio-Rad, AG® 1-X2 Resin, 200-400 mesh, chloride form) were used to separate unreacted [³H]-glutamine from [3H]-glutamate. [³H]-glutamate was eluted from the column with 0.1 N HCl and analyzed for radioactivity using Perkin Elmer's TopCount instrument in conjunction with their 96-well LumaPlates (cat #6005173). Total protein measurements were carried out as per manufacturer's instructions using BioRad's Detergent Compatible Protein Assay kit. Counts per minute were converted to fmol and normalized to total protein content. Data are presented as fmol/mg/h.

### Results

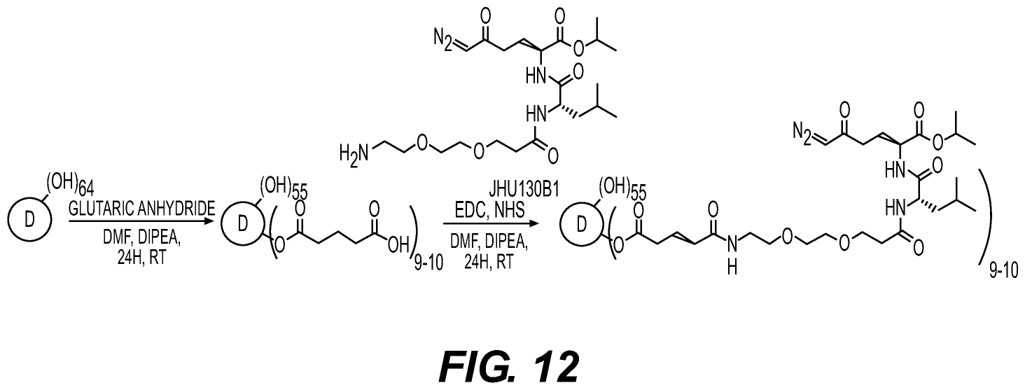

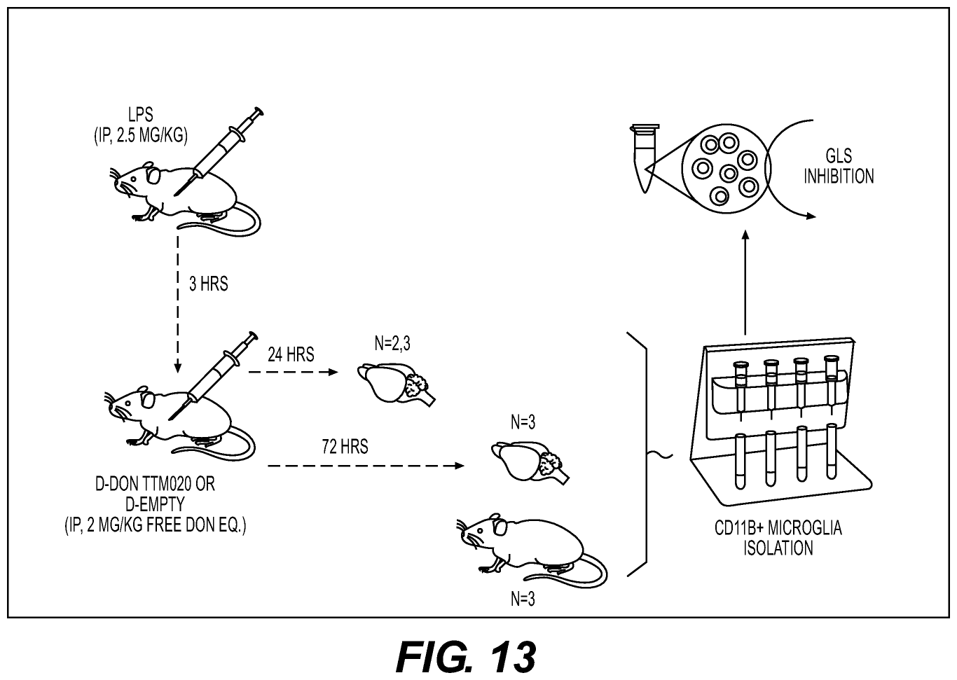

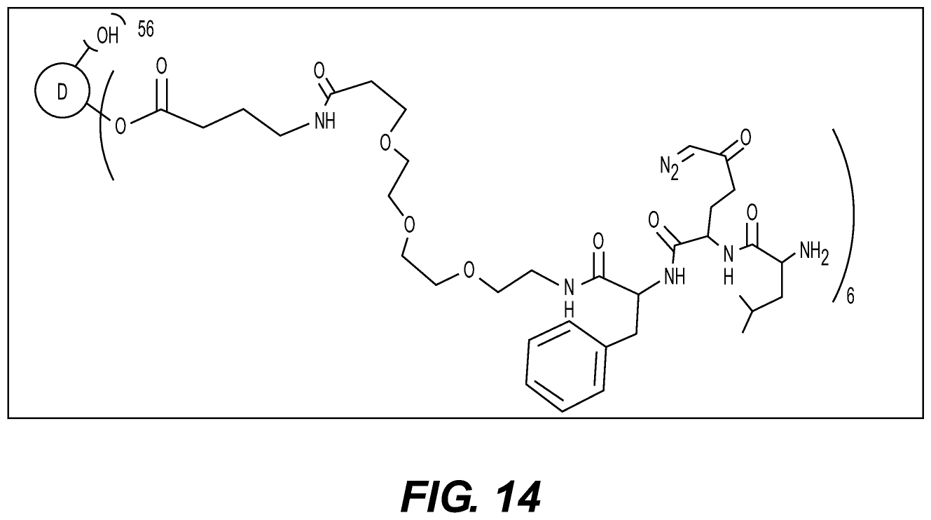

The experimental setup is illustrated in FIG. 12. Dendrimer-TTM020 (FIG. 13) effectively inhibited glutaminase activity compared to unconjugated dendrimer at both 24 hours and 72 hours post drug administration (FIG. 14). Dendrimer conjugated DON analogue, TTM020, provided a sustained glutaminase inhibition in the brain of these mice.

### Example 4: Dendrimer-CY5 Selectively Locates to Brain Tumor Following Systemic Administration

### Methods

### Tumor Inoculations and Treatment Administration

Male and female C57BL/6 mice 6-8 weeks of age were inoculated intracranially with GL261 murine glioblastoma cells obtained from the DTP/DCTD/NCI Tumor Repository (National Cancer Institute, Frederick, MA). GL261 cells were maintained in RPMI with 10% FBS, 1% P/S, and 1% L-glutamine at 37° C. and 5% CO₂ atmosphere. Mice were anesthetized with ketamine (Vedco, St. Joseph, MO) and xylazine (Akorn Animal Health, Lake Forest, IL) cocktail. A midline scalp incision was made, and a burr hole was drilled 1 mm posterior to the bregma and 2 mm lateral to the midline. A 2 μL Hamilton syringe (Hamilton Company, Reno, NV) was lowered to a depth of 2.5 mm to inject 2 μL of GL261 cell solution containing 100,000 cells over 10 min using a stereotactic frame and automated syringe pump (Stoelting Co., Wood Dale, IL). The syringe was withdrawn at 0.5 mm/min and the incision sutured (Ethicon Inc., Somerville, NJ).

To assess the impact of treatment, GL261 brain tumor bearing mice were treated with Dendrimer-KMN045 (FIG. 16) or free DON administered intraperitoneally at a dose of 2 mg/kg (equivalent drug doses for free drug and dendrimer-conjugate) every 4 days starting on day 10 after tumor inoculation. On day 20 after inoculation (or at morbidity if mice met euthanasia criteria prior to day 20), brains were collected. Tumors were dissected out and massed, and tumor burden was calculated as the tumor mass percent out of total brain mass.

### Results

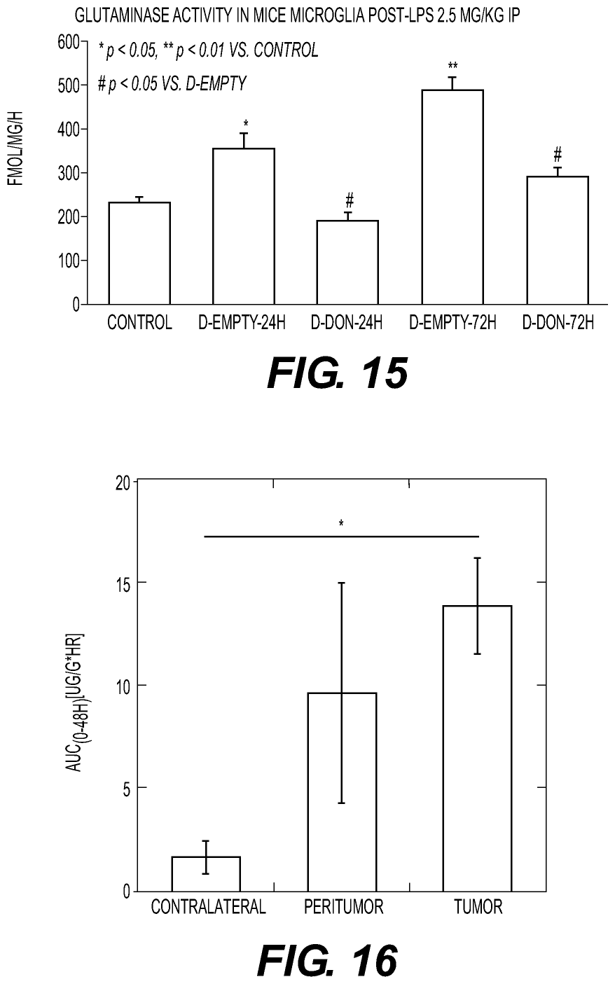

Using confocal microscopy and a rodent tumor-inoculated 9L gliosarcoma model, it has been demonstrated that systemic administration of D-Cy5 (24 hours) resulted in selective brain tumor uptake (FIG. 15). In addition, quantification of D-Cy5 in whole brain showed significant accumulation in tumor versus peritumor and contralateral brain areas. D-Cy5 concentration were quantified from 0 h to 48 h by calculating area under the curve (AUCO-48 h) (p<0.05). Similar selective localization to TAMs was shown in the GL261 mouse model. (Zhang F, et al., Biomaterials. 2015; 52 and Liaw K, et al., Bioengineering & Translational Medicine. 2020).

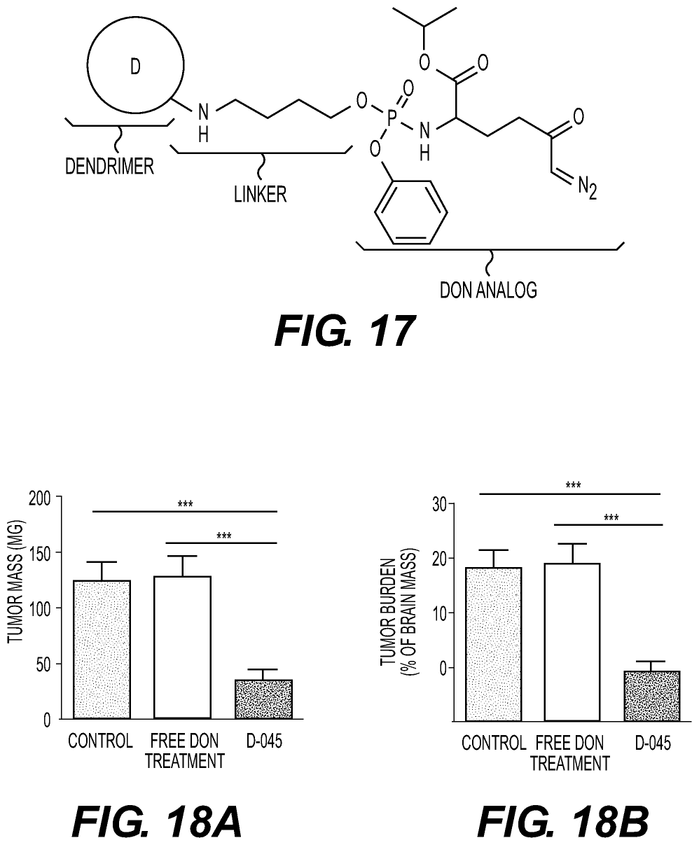

The glutamine antagonist dendrimer conjugate, D-045, showed better efficacy versus free DON when tested head-to-head in a GL261 murine model of GBM in C57BL/6 mice (FIGS. 17A and 17B). The conjugate released <20% of the active drug (FIG. 18). These data provided proof of concept.

### Example 5: Dendrimer-DON (TTM020) Shows DON Release in Tumor Homogenate In Vitro and Shows Complete Regression of Tumor Growth without any Overt Side Effects on Body Weight In Vivo

### Methods

C57BL/6/EL4 tumor-bearing mice were used for efficacy studies following D-DON administration by i.p. route. Briefly, EL4 cells were injected s.c. (0.3×10⁶) and tumor growth was monitored. For treatment the animals with a mean tumor volume of approximately 400 mm³ were randomized into two groups—vehicle and D-DON (2 mg/kg DON equivalent dose; n=7/group). The animals were dosed for 3 days/week for two weeks with simultaneous recording of the tumor volume using Vernier calipers (VWR, USA), body weight, and mortality. The study was continued until complete regression of the tumor.

### Results

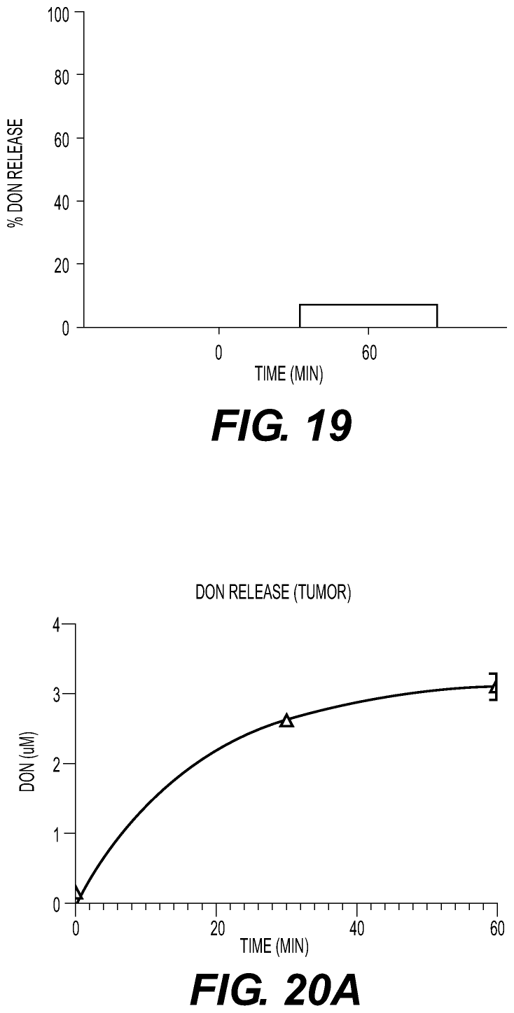

Dendrimer-DON (TTM020) incubated in tumor homogenate showed time-dependent release of DON (FIG. 19A). DDON treatment not only showed tumor growth suppression but also complete regression of tumors. (FIG. 19B). No effect was observed body weight upon treatment suggesting Dendrimer-DON was well-tolerated (FIG. 19C).

It has been established that systemic administration of a dendrimer-glutamine antagonist conjugate provides sustained glutamine antagonist drug levels in activated immune cells, permitting significant dose reduction leading to a substantial improvement in the therapeutic index, making it feasible to bring this therapeutic strategy into clinical development.

### Example 6: Dendrimer-Cy5 is Engulfed by Activated Microglia in Mice after Chronic Social Defeat Stress (CSDS)

### Methods

C57BL/6 mice after 10 days of CSDS (or no CSDS) were injected with 50 mg/kg of D-Cy5 and sacrificed 24 h later via transcardial perfusion of PBS. Brains were post-fixed in 4% paraformaldehyde for 24 h, frozen at −80° C., sectioned at a thickness of 30 μm, and stained for microglia (Iba1), astrocyte (Aldh111), and nuclei (DAPI).

### Results

Using a Cy5 fluorescently labeled PAMAM dendrimer (D-Cy5) synthesized and characterized as previously described (Iezzi, R. et al., Biomaterials. 2012, 33 (3), 979-988; Lesniak, W. G. et al., Mol Pharm. 2013, 10 (12), 4560-4571), its brain penetration in mice after CSDS was tested. It was observed that activated microglia in the mice after CSDS selectively engulfed the dendrimer. Positive D-Cy5 signals were observed near the dentate gyrus of the hippocampus of mice after CSDS, overlapped with Iba1 staining, indicating microglia uptake. D-Cy5 signals were not observed in astrocytes (Aldh111). Brains from non-CSDS mice did not have any positive signal.

### Example 7: Dendrimer-TTM020 Inhibits Microglial Glutaminase Activity in Mice Subjected to CSDS

### Methods

After establishing CSDS, mice were orally treated with D-TTM020 (20 mg/kg), and microglia-enriched CD11b+ cells were isolated from the hippocampus 24 hours after the administration. The protein was then extracted from these cells, and glutaminase activity was measured.

### Results

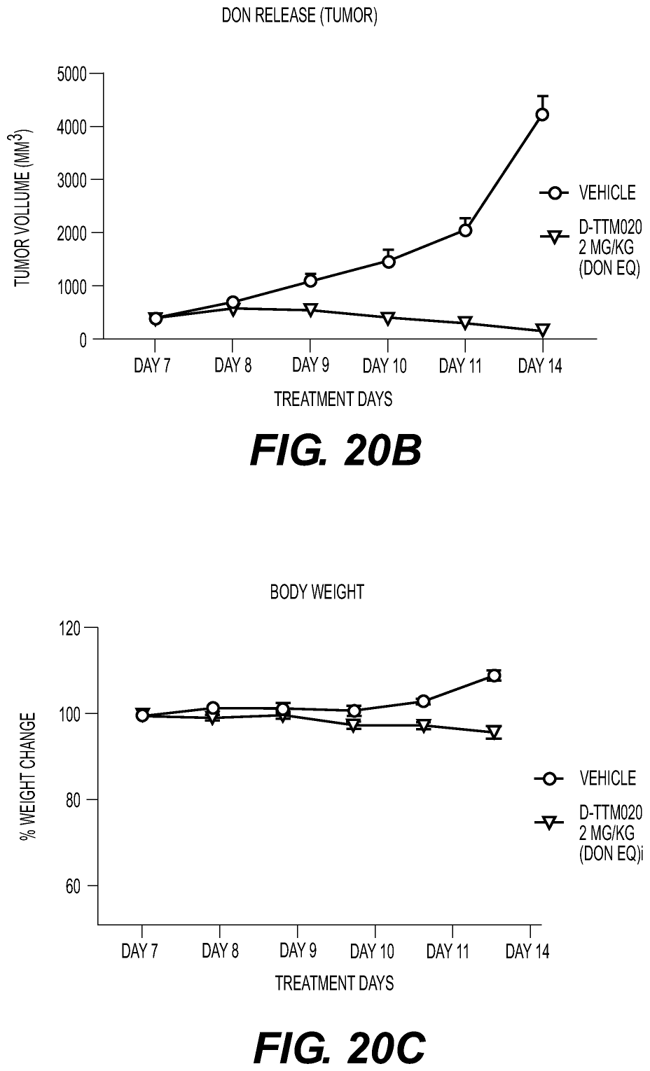

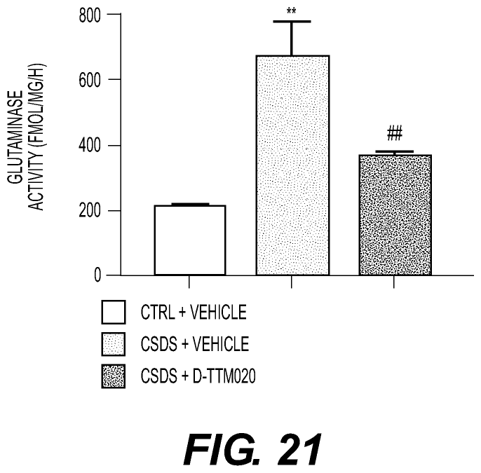

Consistent with our previous report (Zhu, X. et al., Neuropsychopharmacology. 2019, 44 (4), 683-694), mice exposed to CSDS exhibited a significant increase in glutaminase activity. D-TTM020 attenuated up-regulated glutaminase activity in the microglia-enriched CD11b+ cells from CSDS mice (FIG. 20), indicating successful functional target engagement.

Previously reports show that glutamine antagonism inhibits the stress-induced increase in microglial glutaminase activity, inflammatory cytokine production, and normalizes social avoidance and anhedonia induced by Chronic Social Defeat Stress (CSDS), a well-established rodent model used to study stress-induced psychiatric disorders, including depression (Zhu, X. et al., Neuropsychopharmacology. 2019, 44 (4), 683-694). Although glutamine antagonism shows robust therapeutic efficacy, its chronic dosing is known to cause gastrointestinal toxicity limiting its translational application. Given its significant clinical potential, this limitation was addressed by directly targeting the glutamine antagonist to the inflamed brain using a hydroxyl-dendrimer nanoparticles delivery system. Systemic administration of Dendrimer-TTM020 (D-TTM020) provides sustained brain drug levels while rapidly clearing from the periphery, leading to a substantial improvement in its tolerability and maintaining its robust efficacy in stress-associated psychosocial behavior deficits.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A composition comprising dendrimers complexed, covalently conjugated, or intra-molecularly dispersed or encapsulated with one or more 6-diazo-5-oxo-L-norleucine (DON) analogs having one of the following three general chemical formulae, wherein “AA” refers to an amino acid: 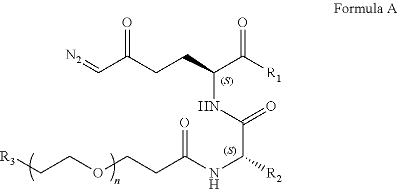
where R₁ is selected from —OCH₂CH₃, —OCH(CH₃)₂—OC(CH₃)₃, —NH₂, NHCH₃, —NH-AA-COOCH(CH₃)₂, and —NH-AA-COOC(CH₃)₃;
where R₂ is selected from —H, —CH₃, —CH₂phenyl, —CH₂CH(CH₃)₂, —CH₂ (indol-3-yl), and —CH₂CH₂C(═O) CH═N≡N;
where R₃ is selected from —NH₂ and —OH; and
n is an integer value from 1 to 32, 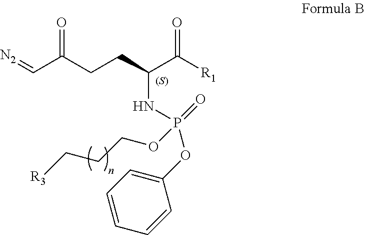
where R₁ is selected from —OCH₂CH₃, —OCH(CH₃)₂—OC(CH₃)₃, —NH₂, NHCH₃, —NH-AA-COOCH(CH₃)₂, and —NH-AA-COOC(CH₃)₃;
where R₃ is selected from —NH₂ and —OH; and
n is an integer value from 1 to 16, 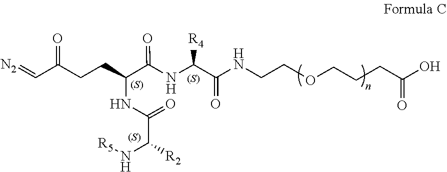
where R₂ is selected from —H, —CH₃, —CH₂phenyl, —CH₂CH(CH₃)₂, —CH₂ (indol-3-yl), or —CH₂CH₂C(═O) CH═N≡N;
where R₄ is selected from —H, —CH₃, —CH₂phenyl, or —CH₂CH(CH₃)₂;
where R₅ is selected from —H, —Ac, —Fmoc, dimethylglycine, Ac-AA-C(═O)—, dimethylglycine-NH-AA-C(═O)—, dimethylglycine-NH-DON—C(═O)—, or C₈H₁₇O—C(═O)CH(NH₂)CH₂CH₂C(═O)—NH—6-diazo-5-oxo-L-norleucine-C(═O)—; and
n is an integer value from 1 to 32.

2. The composition of claim 1, wherein the DON analog is a compound having a structure selected from the group consisting of:

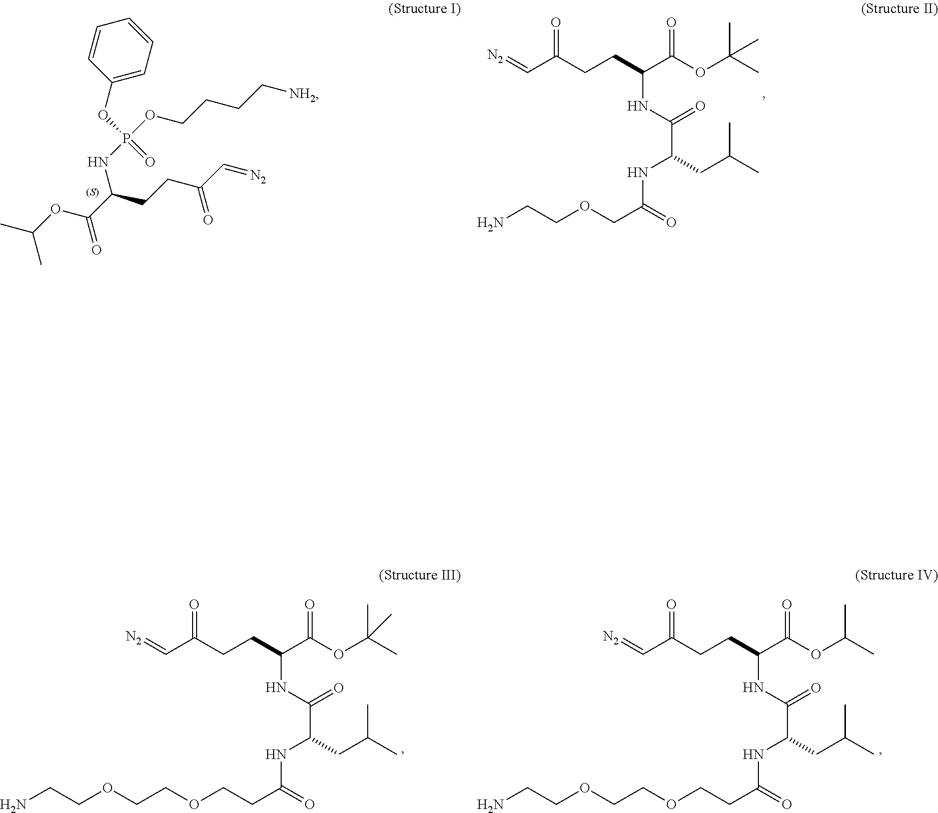

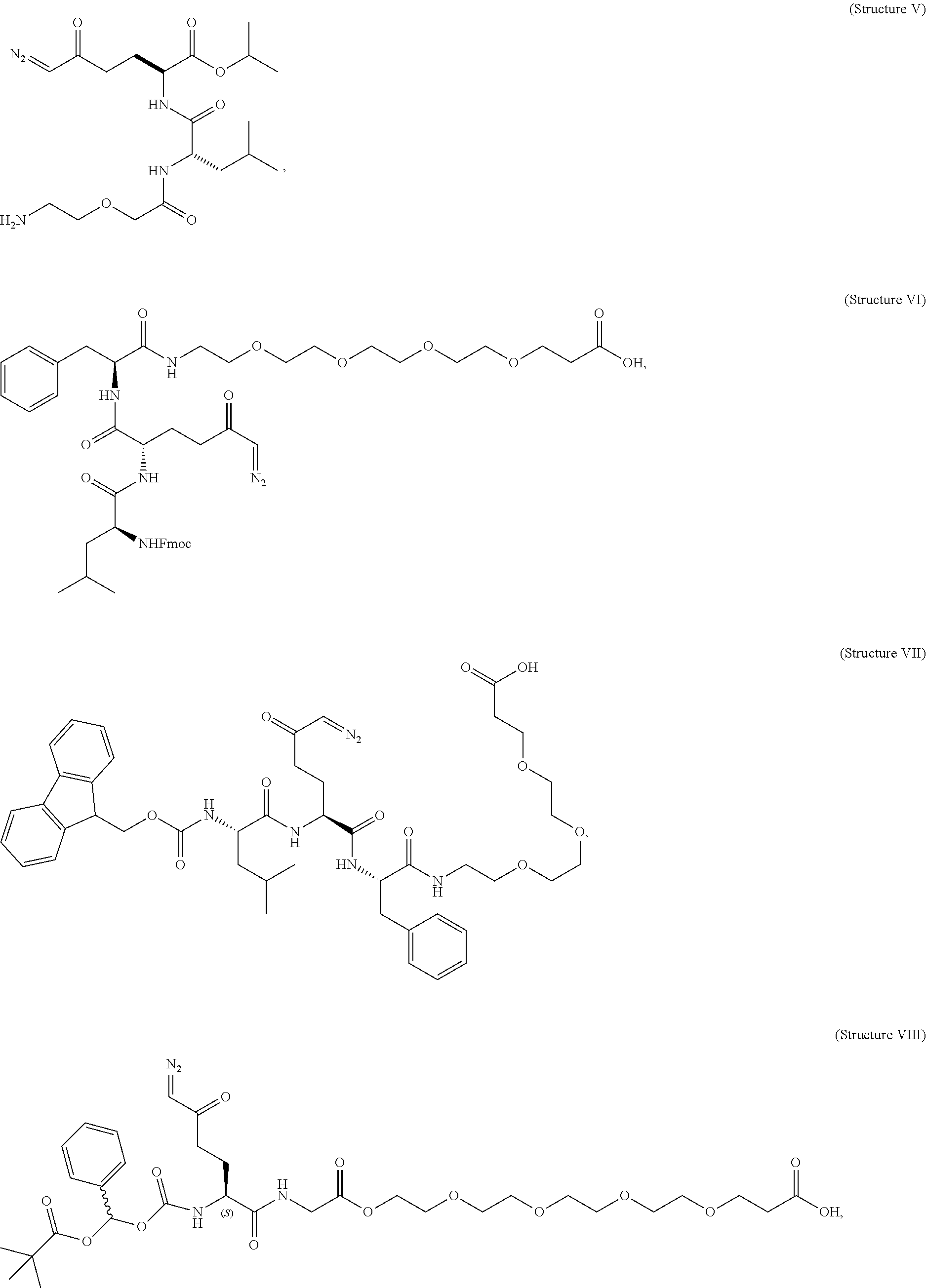

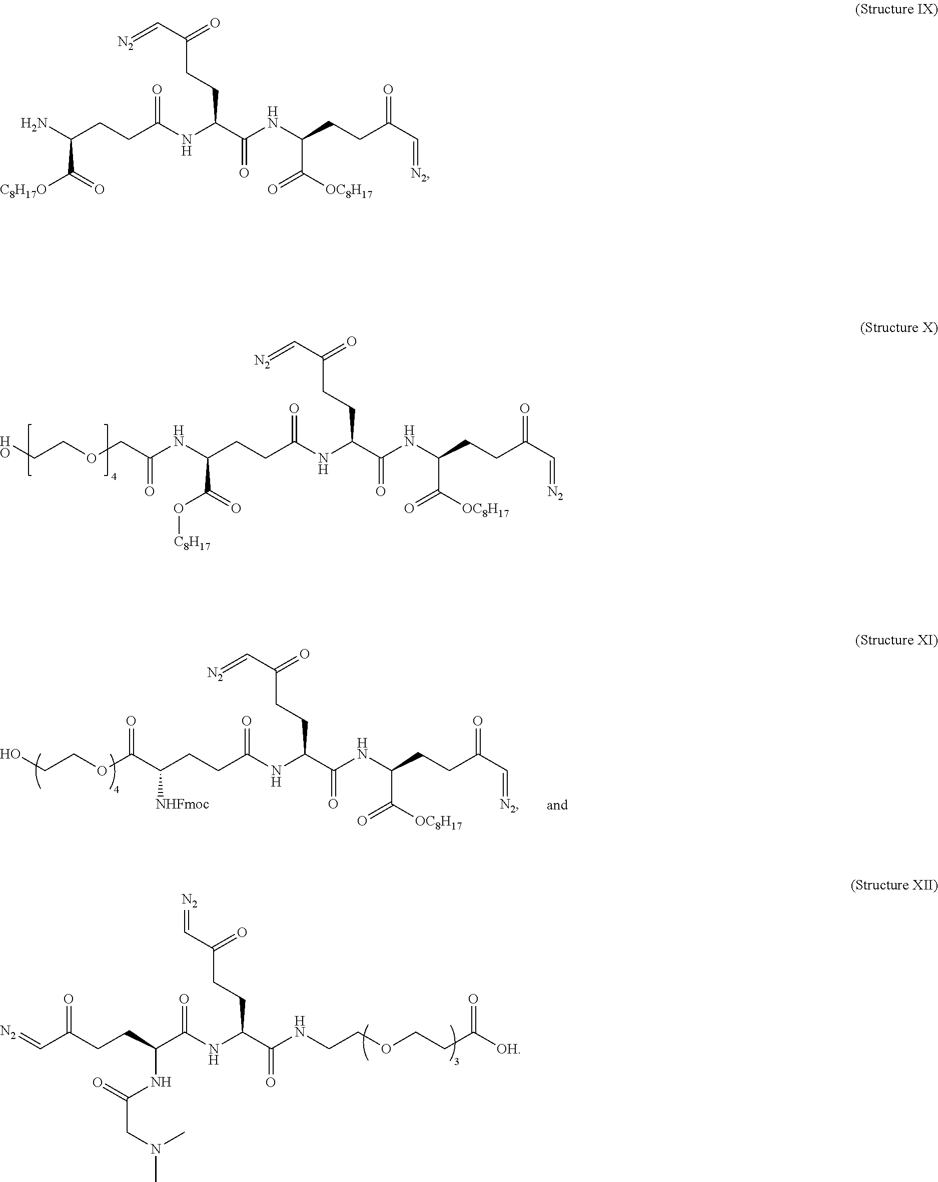

3. The composition of claim 1, wherein the dendrimers are generation 4, generation 5, generation 6, generation 7, or generation 8 dendrimers.

4. The composition of claim 1, wherein the dendrimers are poly(amidoamine) (PAMAM) dendrimers.

5. The composition of claim 1, wherein the dendrimers are hydroxyl-terminated PAMAM dendrimers.

6. The composition of claim 1, wherein the dendrimers are generation 4, generation 5, or generation 6, hydroxyl-terminated PAMAM dendrimers.

7. The composition of claim 1, wherein the dendrimers are covalently conjugated to the one or more DON analogs, optionally via a linker or spacer moiety.

8. The composition of claim 1, wherein the dendrimers are further complexed or conjugated with one or more therapeutic, prophylactic or diagnostic agents.

9. The composition of claim 8, wherein the dendrimers are further complexed or conjugated with one or more neuroprotective agents, anti-inflammatory agents, and/or chemotherapeutic agents.

10. A pharmaceutical composition comprising the composition of claim 1, and one or more pharmaceutically acceptable excipients.

11. The pharmaceutical composition of claim 10, formulated for parenteral or oral administration.

12. The pharmaceutical composition of claim 10, formulated in a form selected from the group consisting of hydrogels, nanoparticle or microparticles, suspensions, powders, tablets, capsules, and solutions.

13. A method for treating one or more of neurological, oncological, and immune disorders in a subject in need thereof comprising administering to the subject an effective amount of the composition of claim 1 to treat, alleviate, and/or prevent one or more symptoms associated with the one or more of neurological, oncological, and immune disorders.

14. The method of claim 13, wherein the composition is administered in an amount effective to decrease glutaminase activity and/or reduce glutamate release in one or more target cells associated with the diseased or injured tissue of one or more of neurological, oncological, and immune disorders.

15. The method of claim 13, wherein the composition is administered in an amount effective to decrease glutaminase activity and/or reduce glutamate release in activated microglia associated with the diseased or injured tissue of one or more of neurological, oncological, and immune disorders.

16. The method of claim 13, wherein the neurological disorder is selected from the group consisting of stress-induced mood disorders, cognitive deficit, ischemia, neuroinflammation, Alzheimer's disease, and multiple sclerosis.

17. The method of claim 13, wherein the composition is administered in an amount effective to reduce neuroinflammation, improve cognition, or a combination thereof.

18. The method of claim 13, wherein the oncological disorder is selected from the group consisting of breast cancer, ovarian cancer, uterine cancer, prostate cancer, testicular germ cell tumor, brain cancer, gastric cancer, esophagus cancer, lung cancer, liver cancer, renal cell cancer, colorectal cancer, and pancreatic cancer.

19. The method of claim 13, wherein the composition is administered in an amount effective to reduce tumor size.

20. The method of claim 13, further comprising administering to the subject one or more selected from the group consisting of an immune checkpoint modulator, a chemotherapeutic agent, an anti-infective agent, adoptive T cell therapy, a cancer vaccine, surgery, and radiation therapy.

21. The method of claim 20, wherein the immune checkpoint modulator is selected from the group consisting of PD-1 antagonists, PD-1 ligand antagonists, and CTLA4 antagonists.

22. The method of claim 13, wherein the immune disorder is selected from the group consisting of rheumatoid arthritis, psoriasis, psoriatic arthritis, systemic lupus erythematosus (SLE), type 1 diabetes, inflammatory bowel disease, and thyroid diseases.

23. A method for inhibiting activities of glutaminase in activated microglia associated with a diseased or injured tissue of a subject comprising systemically administering to the subject an effective amount of the composition of claim 1.

24. The method of claim 13, wherein the composition is administered orally or parenterally.

25. The method of claim 13, wherein the composition is administered intravenously.

26. A molecule having a structure selected from the group consisting of 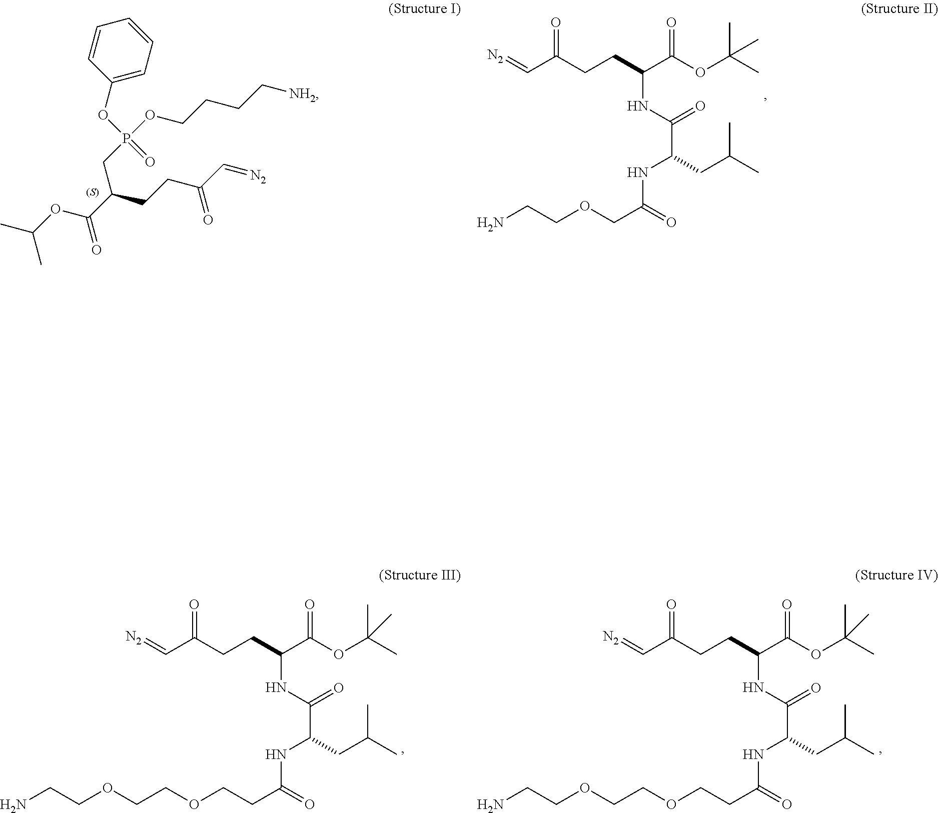 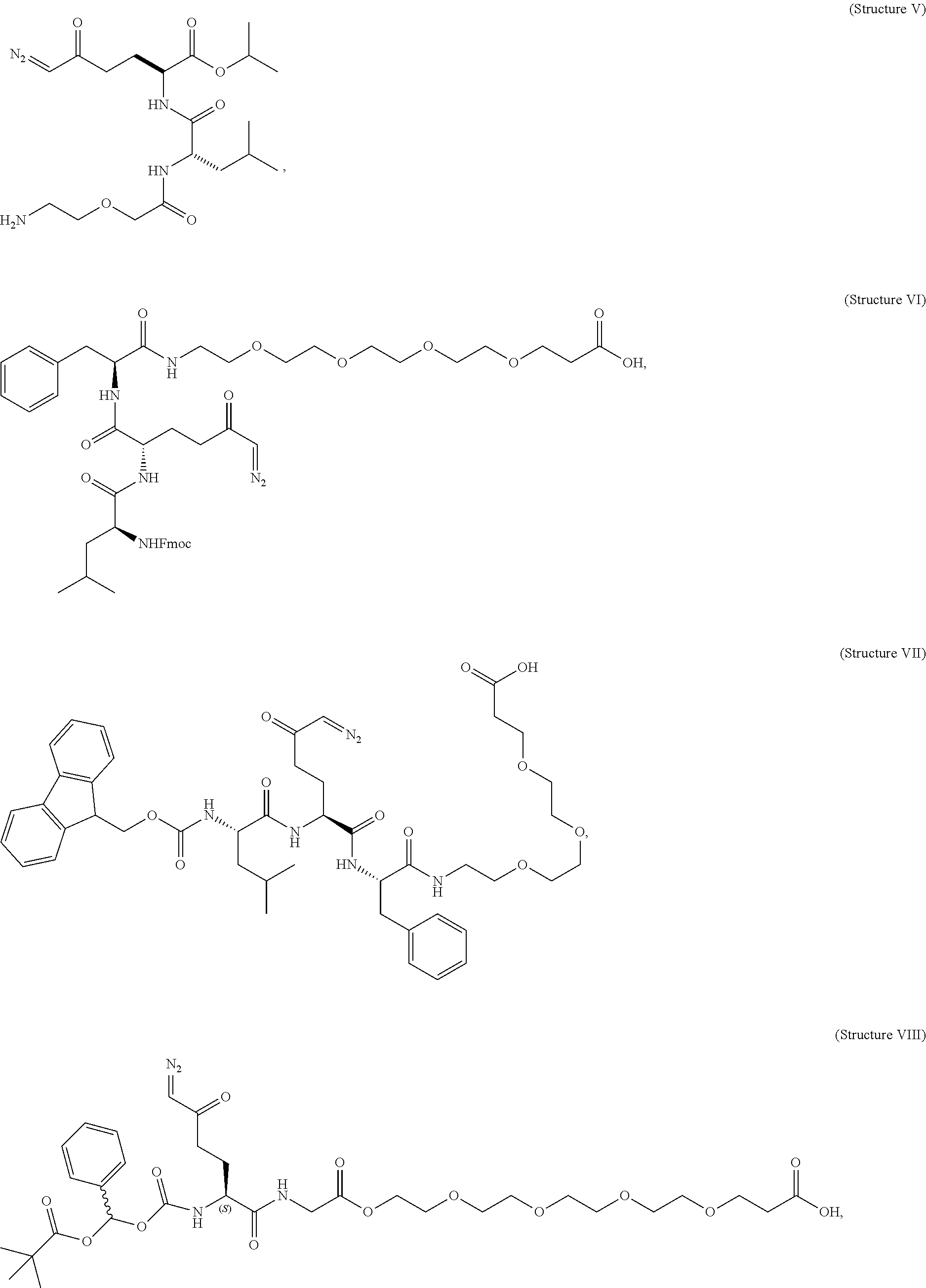 [chemical structure]
